(19) United States
(12) Patent Application Publication
(10) Pub. No.: US 2025/0109191 A1
(43) Pub. Date: Apr. 3, 2025

(54) METHODS FOR ALTERING BODY COMPOSITION BY ADMINISTERING A GDF8 INHIBITOR AND AN ACTIVIN A INHIBITOR

(71) Applicant: REGENERON PHARMACEUTICALS, INC., Tarrytown, NY (US)

(72) Inventors: Stephen DONAHUE, Princeton, NJ (US); Robert C. PORDY, Ardsley, NY (US); Gary HERMAN, Princeton, NJ (US)

(21) Appl. No.: 18/910,740

(22) Filed: Oct. 9, 2024

Related U.S. Application Data
(63) Continuation of application No. 17/570,128, filed on Jan. 6, 2022, now abandoned.
Application No. 17/570,128 is a continuation of application No. 16/290,287, filed on Mar. 1, 2019, now Pat. No. 11,248,044.
(60) Provisional application No. 62/637,017, filed on Mar. 1, 2018.

(51) Int. Cl. C07K 16/22; A61K 9/00; A61K 39/00; A61K 39/395; A61P 3/04; A61P 21/00; A61P 21/06

(52) U.S. Cl. CPC C07K 16/22; A61K 39/3955; A61P 3/04; A61P 21/00; A61P 21/06; A61K 9/0019; A61K 9/0053; A61K 2039/505; A61K 2039/507; A61K 2039/54; A61K 2039/545; A61K 2300/00; C07K 2317/76

(57) ABSTRACT

The present invention relates to compositions and methods for altering body composition in a subject, wherein the alteration of body composition is an increase in muscle mass and a reduction of fat mass simultaneously. The present invention also relates to compositions and methods for reducing fat mass in a subject. The compositions and methods also increase muscle volume and lean body mass in the subject. The present invention also relates to compositions that comprise a GDF8 inhibitor and an Activin A inhibitor and the use of such compositions to treat diseases and disorders characterized by increased fat mass, and/or decreased muscle volume.

### CROSS REFERENCES TO RELATED APPLICATIONS

This application is a continuation of U.S. Non-Provisional application Ser. No. 17/570,128, filed on 6 Jan. 2022, now abandoned, which is a continuation of U.S. Non-Provisional application Ser. No. 16/290,287, filed on 1 Mar. 2019, now issued as U.S. Pat. No. 11,248,044, issued on 15 Feb. 2022, which claims the benefit of U.S. Provisional Application No. 62/637,017, filed on 1 Mar. 2018, the entire contents of which are incorporated herein by reference.

### SEQUENCE LISTING

This application includes a Sequence Listing in electronic XML format entitled “40848_0091USC2_SL.xml”, which was created on 26 Nov. 2024 and which has a size of 574,457 bytes. The contents of XMLfile “40848_0091USC2_SL.xml” are incorporated by reference herein.

### FIELD OF THE INVENTION

The present invention relates to compositions and methods for altering body composition in a subject. The compositions and methods also reduce fat mass in a subject. The compositions and methods also increase muscle volume and/or lean body mass in the subject. More specifically, the invention relates to compositions that comprise a GDF8 inhibitor and an Activin A inhibitor and the use of such compositions to treat diseases and disorders characterized by increased fat mass, and/or decreased muscle volume or decreased lean body mass.

### BACKGROUND

Growth and differentiation factor-8 (GDF8, also known as myostatin), is a secreted ligand belonging to the transforming growth factor-β (TGF-β) superfamily of growth factors. GDF8 plays a central role in the development and maintenance of skeletal muscle, acting as a negative regulator of myogenesis and skeletal muscle mass. Myostatin mutations (including knockout) translate into phenotypes that are predominantly increased muscle mass, but can be made up of variations in muscling (more muscle fibers), muscle fiber composition (greater cross-sectional area of muscle fibers), increased protein/DNA ratio, and other.

Antibodies to GDF8 and therapeutic methods are disclosed in, e.g., U.S. Pat. No. 8,840,894. Anti-GDF8 antibodies are also mentioned in, e.g., U.S. Pat. Nos. 6,096,506; 7,320,789; 7,261,893; 7,807,159; 7,888,486; 7,635,760; 7,632,499; in US Patent Appl. Publ. Nos. 2006/0263354; 2007/0178095; 2008/0299126; 2010/0166764; 2009/0148436; and International Patent Appl. Publ. Nos. WO2004/037861; WO2007/047112; WO 2010/070094.

Activins belong to the transforming growth factor-beta (TGF-β) superfamily and exert a broad range of biological effects on cell proliferation, differentiation, metabolism, homeostasis, and apoptosis, as well as immune response and tissue repair. Activin A is a disulfide-linked homodimer (two beta-A chains) that binds to and activates heteromeric complexes of a type I (Act RI-A and Act RI-B) and a type II (Act RII-A and Act RII-B) serine-threonine kinase receptor.

Antibodies to Activin A and uses thereof are disclosed in, e.g., U.S. Pat. Nos. 8,309,082; 9,718,881; and International Patent Appl. Publ. No. WO2008/031061.

Compositions comprising an anti-GDF8 antibody and an anti-Activin A antibody and therapeutic methods are disclosed in, e.g., U.S. Pat. No. 8,871,209.

Obesity is a global problem for over a third of the world population. In the United States of America, the average obesity rate is over 20%. The costs of obesity-related illness are staggering, amounting to $190.2 billion, roughly 21% of annual medical costs in the U.S. Obesity is an epidemic disease characterized by chronic low-grade inflammation associated with dysfunctional (elevated) fat mass. In Framingham Heart Study participants, abdominal adiposity was associated with incident cardiovascular disease (CVD) after adjustments for clinical risk factors and overall adiposity. Britton JACC 2013 62; 921. Abdominal visceral fat accumulation was positively associated with the progression of coronary noncalcified plaque. Imai Atherosclerosis 2012. Because high fat mass is associated with such serious conditions as congestive heart failure, high blood pressure/hypertension, pulmonary embolism, osteoarthritis, lymphedema, gastro-esophageal reflux disease, chronic renal failure, cancer, fatty-liver disease, and even depression, there remains a need for therapies that reduce total fat and/or android fat mass in subjects.

### BRIEF SUMMARY OF THE INVENTION

In one aspect, the invention is directed to a method for altering body composition in a subject, i.e., increase muscle mass and decreasing fat mass, comprising administering a first composition comprising an effective amount of a GDF8 inhibitor and a second composition comprising an effective amount of an Activin A inhibitor to the subject. In another aspect, the invention is directed to a method for inducing a reduction in fat mass in a subject comprising administering an effective amount of a composition comprising a GDF8 inhibitor and an Activin A inhibitor to the subject.

In another aspect, the invention is directed to a method for treating a disease or disorder characterized by or associated with increased fat mass, the method comprising administering to a subject in need thereof a first composition comprising an effective amount of a GDF8 inhibitor and a second composition comprising an effective amount of an Activin A inhibitor. In another aspect, the invention is directed to a method for treating a disease or disorder characterized by or associated with increased fat mass, the method comprising administering to a subject in need thereof an effective amount of a composition comprising a GDF8 inhibitor and an Activin A inhibitor. In one aspect, the invention is directed to a method for altering body composition in a subject, i.e., increase muscle mass and decreasing fat mass, comprising administering an effective amount of a GDF8 inhibitor and an effective amount of an Activin A inhibitor to the subject, wherein there is an insignificant change in total body mass. Thus, in one aspect of the invention, a subject administered an effective amount of a GDF8 inhibitor and an effective amount of an Activin A inhibitor will experience an increase in muscle mass concurrently with a decrease in fat mass, leading to a minimal and/or insignificant change in total body mass.

In one aspect, the invention is directed to a use of a GDF8 inhibitor and/or an Activin A inhibitor in the preparation of a medicament for achieving a reduction in fat mass in a subject. In another aspect, the invention is directed to a use of a GDF8 inhibitor and/or an Activin A inhibitor in the preparation of a medicament for treating a disease or disorder associated with increased fat mass in a subject.

In some embodiments, a GDF8 inhibitor is provided for use in a method for treating a disease or disorder characterized by increased fat mass, wherein the method comprises administrating to a subject the GDF8 inhibitor and an Activin A inhibitor.

In some embodiments, an Activin A inhibitor is provided for use in a method for treating a disease or disorder characterized by increased fat mass, wherein the method comprises administrating to a subject the Activin A inhibitor and a GDF8 inhibitor.

In some embodiments, a non-therapeutic method is provided for decreasing fat mass in a subject, the method comprising administering to the subject an Activin A inhibitor and a GDF8 inhibitor.

In one embodiment of a method according to the invention, the effective amount of a GDF8 inhibitor comprises a dosing regimen selected from the group consisting of at least 0.1 mg/kg to about 10 gm/kg, 1 mg/kg to about 1 gm/kg, and 10 mg/kg to 100 mg/kg. In a further embodiment of a method according to the invention, the effective amount of a GDF8 inhibitor comprises a dosing regimen selected from the group consisting of a single dose of about 0.01 to about 20 mg/kg body weight, about 0.1 to about 10 mg/kg body weight, and about 0.1 to about 5 mg/kg body weight.

In another embodiment of a method according to the invention, the effective amount of an Activin A inhibitor comprises a dosing regimen selected from the group consisting of at least 0.1 mg/kg to about 10 gm/kg, 1 mg/kg to about 1 gm/kg, and 10 mg/kg to 100 mg/kg. In a further embodiment of a method according to the invention, the effective amount of an Activin A inhibitor comprises a dosing regimen selected from the group consisting of a single dose of about 0.01 to about 20 mg/kg body weight, about 0.1 to about 10 mg/kg body weight, and about 0.1 to about 5 mg/kg body weight.

In one embodiment of a method according to the invention, the effective amount of a GDF8 inhibitor is 6 mg/kg body weight and the effective amount of an Activin A inhibitor is 3 mg/kg body weight. In one embodiment of a method according to the invention, the effective amount of a GDF8 inhibitor is 6 mg/kg body weight and the effective amount of an Activin A inhibitor is 10 mg/kg body weight.

In one embodiment of a method according to the invention, the first composition is formulated for intravenous, subcutaneous, or oral administration. In another embodiment of a method according to the invention, the second composition is formulated for intravenous, subcutaneous, or oral administration. In certain embodiments of a method according to the invention, the first and second compositions are administered concurrently or sequentially to the subject.

In one embodiment of a method according to the invention, the first and second compositions are combined into a third composition prior to administration. In a further embodiment, the third composition is formulated for intravenous, subcutaneous, or oral administration.

In one embodiment, a method according to the invention further comprises measuring total fat mass in the subject before administration. In another embodiment, a method according to the invention further comprises measuring total fat mass in the subject after administration, and administering the first and second composition until the subject has a reduction in total fat mass of at least 2% to 8%, 2.5% to 6%, 3% to 4%, or at least 2.0%, at least 2.5%, at least 3.0%, or at least 3.5%, or more.

In one embodiment, a method according to the invention further comprises measuring android fat mass in the subject before administration. In another embodiment, a method according to the invention further comprises measuring android fat mass in the subject after administration, and administering the first and second composition until the subject has a reduction in android fat mass of at least 2% to 8%, 2.5% to 6%, 3% to 4%, or at least 2.0%, at least 2.5%, at least 3.0%, or at least 3.5%, or more.

In one embodiment, a method according to the invention further comprises measuring subcutaneous adipose tissue volume in the subject before administration. In another embodiment, a method according to the invention further comprises measuring subcutaneous adipose tissue volume in the subject after administration, and administering the first and second composition until the subject has a reduction in android fat mass of at least 2% to 8%, 2.5% to 6%, 3% to 4%, or at least 2.0%, at least 2.5%, at least 3.0%, or at least 3.5%, or more.

In some embodiments, a method is provided comprising administering to a subject in need thereof an effective amount of a GDF8 inhibitor and an effective amount of a Activin A inhibitor, wherein the GDF8 inhibitor and the Activin A inhibitor are co-administered within 48 hours or less, 24 hours or less, 12 hours or less, 6 hours or less, 3 hours or less, or 1 hour or less.

In some embodiments, a method is provided comprising administering to a subject in need thereof an effective amount of a GDF8 inhibitor and an effective amount of a Activin A inhibitor, wherein the subject exhibits a decrease in total fat mass, android fat mass, and/or subcutaneous adipose tissue volume.

In some embodiments, a method is provided comprising administering to a subject in need thereof an effective amount of a GDF8 inhibitor and an effective amount of a Activin A inhibitor, wherein the subject exhibits a decrease in total fat mass, android fat mass, and/or subcutaneous adipose tissue volume after 4 weeks or more, or 8 weeks or more, following administration.

In some embodiments, a method is provided comprising administering to a subject in need thereof an effective amount of a GDF8 inhibitor and an effective amount of a Activin A inhibitor, wherein the subject exhibits a decrease in total fat mass, android fat mass, and/or subcutaneous adipose tissue volume, wherein the subject does not exhibit reduced thigh intramuscular adipose tissue volume.

In some embodiments, a method is provided comprising administering to a subject in need thereof an effective amount of a GDF8 inhibitor and an effective amount of a Activin A inhibitor, wherein the subject exhibits a decrease in total fat mass, android fat mass, and/or subcutaneous adipose tissue volume, wherein the subject does not exhibit reduced thigh intramuscular adipose tissue volume after 4 weeks or more, or 8 weeks or more, following administration.

In some embodiments, a kit is provided comprising a first container containing an effective amount of a GDF8 inhibitor and a second container containing an effective amount of a specific Activin A inhibitor.

In some embodiments, the GDF8 inhibitor is an isolated antibody or an antigen-binding fragment thereof that specifically binds to GDF8.

In one embodiment of a method according to the invention, the GDF8 inhibitor is an antibody or an antigen-binding fragment thereof that specifically binds to GDF8. In another embodiment, the antibody or antigen-binding fragment that specifically binds GDF8 comprises the heavy chain complementarity determining regions (HCDRs) of a heavy chain variable region (HCVR) comprising SEQ ID NO:360, and the light chain complementarity determining regions (LCDRs) of a light chain variable region (LCVR) comprising SEQ ID NO: 368. In still another embodiment, the antibody or antigen-binding fragment that specifically binds GDF8 comprises three HCDRs comprising SEQ ID NO:362, SEQ ID NO: 364, and SEQ ID NO:366, and three LCDRs comprising SEQ ID NO:370, TTS, and SEQ ID NO:374.

In some embodiments, the Activin A inhibitor is an isolated antibody or an antigen-binding fragment thereof that specifically binds to Activin A.

In one embodiment of a method according to the invention, the Activin A inhibitor is an antibody or antigen-binding fragment thereof that specifically binds Activin A. In another embodiment, the antibody or antigen-binding fragment that specifically binds Activin A comprises the heavy chain complementarity determining regions (HCDRs) of a heavy chain variable region (HCVR) comprising SEQ ID NO:553, and the light chain complementarity determining regions (LCDRs) of a light chain variable region (LCVR) comprising SEQ ID NO: 537. In still another embodiment, the antibody or antigen-binding fragment that specifically binds Activin A comprises three HCDRs comprising SEQ ID NO:555, SEQ ID NO: 557, and SEQ ID NO:559, and three LCDRs comprising SEQ ID NO:539, GAS, and SEQ ID NO:543.

In one embodiment of a method according to the invention, the effective dose of the Activin A inhibitor is selected from the group comprising between 100% to 200% of the effective dose of the GDF8 inhibitor, between 100% and 250% of the effective dose of the GDF8 inhibitor, between 100% and 300% of the effective dose of the GDF8 inhibitor, and between 100% and 400% by weight of the effective dose of the GDF8 inhibitor.

In another embodiment of a method according to the invention, the weight ratio of the effective dose of the Activin A inhibitor to effective dose of the GDF8 inhibitor is from 10:1 to 1:10, 8:1 to 1:8, 6:1 to 1:6, 3:1 to 1:3, or about 2:1 to 1:2. In another embodiment of a method according to the invention, the weight ratio of the effective dose of the Activin A inhibitor is about 1.5 to 2.0 times as large by weight as the amount the GDF8 inhibitor.

In one embodiment of a method according to the invention, the GDF8 inhibitor is a bispecific antibody or antigen-binding fragment thereof that specifically binds GDF8 and also specifically binds Activin A. In another embodiment, the Activin A inhibitor is a bispecific antibody or antigen-binding fragment thereof that specifically binds Activin A and also specifically binds GDF8.

In one embodiment of a method according to the invention, the reduction of fat mass in the subject is a reduction in total fat mass as measured by DXA (Dual-energy X-ray absorptiometry). In another embodiment of a method according to the invention, the reduction of fat mass in the subject is a reduction in android fat mass as measured by DXA (Dual-energy X-ray absorptiometry).

In one embodiment of a method according to the invention, the reduction of fat mass in the subject is a reduction in subcutaneous adipose tissue volume as measured by MRI (Magnetic Resonance Imaging).

In one embodiment of a method according to the invention, the subject experiences an increase in muscle volume. The muscle volume may be thigh muscle tissue volume, for example, as measured by MRI. In some embodiments, the muscle volume may be thigh muscle tissue volume, for example, as measured by MRI. In some embodiments, the thigh muscle volume may be thigh muscle tissue volume including intramuscular adipose tissue and large vessels, or thigh muscle tissue volume excluding intramuscular adipose tissue and large vessels, for example, as measured by MRI.

In one embodiment of a method according to the invention, the subject experiences an increase in total lean mass. The total lean mass may be measured by DXA (dual x-ray absorptiometry.

In one embodiment of a method according to the invention, the subject experiences an increase in appendicular lean body mass. The appendicular lean body mass may be measured by DXA, and, for example, calculated by aLBM equation.

In one embodiment of a method according to the invention, the subject experiences a decrease in total fat mass, for example, as measured by DXA.

In one embodiment of a method according to the invention, the subject experiences a decrease in android fat mass, for example, as measured by DXA.

In one embodiment of a method according to the invention, the subject experiences a decrease in subcutaneous adipose tissue volume, for example, as measured by DXA.

In one embodiment of a method according to the invention, the subject experiences a decrease in sum of fat mass of arms and legs, for example, as measured by DXA.

In one embodiment of the invention, the subject does not exhibit a decrease in thigh intramuscular adipose tissue volume, for example, as measured by MRI.

In one embodiment of the invention, the subject does not exhibit a decrease in total bone mineral density (BMD) mass, for example, as measured by DXA.

In one embodiment of the invention, the subject does not exhibit a decrease in total bone mineral content (BMC) mass, for example, as measured by DXA.

In one embodiment of the invention, the subject exhibits an increase in total bone mineral content (BMC) mass, for example, as measured by DXA.

In another embodiment of a method according to the invention, the subject does not have a muscle wasting condition or disease.

In some embodiments, a kit is provided for use in altering body composition, decreasing fat mass, increasing lean mass, or treating a disease or disorder characterized by or associated with increased fat mass, the kit comprising a first container comprising a composition comprising an effective amount of a GDF8 inhibitor and a second container comprising a second composition comprising an effective amount of an Activin A inhibitor.

In some embodiments, a GDF8 inhibitor is provided for use in manufacture of a first composition for use as a medicament in a kit for altering body composition, decreasing fat mass, increasing lean mass, or treating a disease or disorder characterized by or associated with increased fat mass, the kit further comprising a second composition comprising an Activin A inhibitor.

In some embodiments, an Activin A inhibitor is provided for use in manufacture of a first composition for use as a medicament in a kit for altering body composition, decreasing fat mass, increasing lean mass, or treating a disease or disorder characterized by or associated with increased fat mass, the kit further comprising a second composition comprising a GDF8 inhibitor.

In some embodiments, a GDF8 inhibitor is provided for use in manufacture of a first composition for use in a kit for altering body composition, decreasing fat mass, or increasing lean mass in a subject, the kit further comprising a second composition comprising an Activin A inhibitor.

In some embodiments, an Activin A inhibitor is provided for use in manufacture of a first composition for use in a kit for altering body composition, decreasing fat mass, or increasing lean mass in a subject, the kit further comprising a second composition comprising a GDF8 inhibitor.

In some embodiments, a first composition comprising a GDF8 inhibitor is provided for use in altering body composition, decreasing fat mass, increasing lean mass, or treating a disease or disorder characterized by or associated with increased fat mass or decreased lean mass in a subject, wherein the subject has received a second composition comprising an Activin A inhibitor.

In some embodiments, a first composition comprising an Activin A inhibitor is provided for use in altering body composition, decreasing fat mass, increasing lean mass, or treating a disease or disorder characterized by or associated with increased fat mass or decreased lean mass in a subject, wherein the subject has received a second composition comprising a GDF8 inhibitor.

In some embodiments, a first composition comprising a GDF8 inhibitor is provided for use in method for altering body composition, decreasing fat mass, increasing lean mass, or treating a disease or disorder characterized by or associated with increased fat mass or decreased lean mass, the method further comprising administering a second composition comprising an Activin A inhibitor.

In some embodiments, a first composition comprising an Activin A inhibitor is provided for use in method for altering body composition, decreasing fat mass, increasing lean mass, or treating a disease or disorder characterized by or associated with increased fat mass or decreased lean mass, the method further comprising administering a second composition comprising an GDF8 inhibitor.

In some embodiments, a composition is provided comprising an Activin A inhibitor and a GDF8 inhibitor for use in altering body composition, decreasing fat mass, increasing lean mass, or treating or preventing a disease or disorder characterized by or associated with increased fat mass or decreased lean mass.

Other embodiments of the present invention will become apparent from a review of the ensuing detailed description.

### BRIEF DESCRIPTION OF THE FIGURES

FIG. 1 shows a bar graph depicting results of clinical studies in sarcopenia patients 70 years of age or older receiving anti-GDF8 antibody REGN1033 alone after 12 weeks as the percent change from baseline of total lean mass in LS mean and SE compared to placebo. Patients receiving REGN1033 exhibited significantly increased total lean body mass at 12 weeks at each of three dosing regimens when compared to placebo (n=65). Patients receiving 100 mg anti-GDF8 antibody REGN1033 Q4W S.C. exhibited a difference vs. placebo of 1.66% total lean mass (n=62, P=0.0077). Patients receiving 300 mg anti-GDF8 Q4W SC exhibited a difference vs. placebo of 1.78% total lean mass (n=64, P=0.0043). Patients receiving 300 mg Q2W SC exhibited a difference vs. placebo of 2.29% total lean mass (n=59, P=0.0004).

FIG. 2A shows Table 1 with the ascending dose panels used in the study of 48 healthy post-menopausal women according to Example 2. A single intravenous dose of anti-GDF8 antibody REGN1033 and/or anti-Activin A antibody REGN2477 was utilized. In the primary analyses, the placebo and high dose combination groups were pooled across panels, yielding 12 subjects on placebo and 12 on the high dose combination, as shown by boxed regions in the table.

FIG. 2B shows a bar graph depicting thigh muscle volume (measured via MRI) % change at week 8 after a single I.V. dose of anti-Activin A antibody REGN2477 and/or anti-GDF8 antibody REGN1033 in healthy post-menopausal women. A significant increase in thigh muscle volume % change from placebo was exhibited by anti-GDF8 (6 mg/kg), anti-GDF8 (6 mg/kg)+mid dose anti-Activin A (3 mg/kg), and anti-GDF8 (6 mg/kg)+high dose anti-Activin A (10 mg/kg) groups. (*nominal p<0.5 vs. placebo, ****nominal p<0.0001 vs. placebo).

FIG. 2C shows a bar graph depicting total fat mass (measured via DXA) % change at week 8 after a single I.V. dose of anti-Activin A antibody REGN2477 and/or anti-GDF8 antibody REGN1033 in healthy post-menopausal women. The numbers show changes from placebo. A significant decrease in total fat mass % change was exhibited by the anti-GDF8 (6 mg/kg)+high dose anti-Activin A (10 mg/kg) group. (*nominal p<0.05 vs. placebo).

FIG. 3 shows a line graph depicting LS mean (SE) percent change from baseline in thigh muscle volume by MRI at weeks 0, 4, and 8 after a single I.V. dose of anti-Activin A antibody REGN2477 and/or anti-GDF8 antibody REGN1033 in healthy post-menopausal women. At 4 weeks after single I.V. dose, a significant increase in thigh muscle volume (measured by MRI excluding intramuscular adipose tissue) % change from placebo was exhibited by each of anti-GDF8 (6 mg/kg), anti-GDF8 (6 mg/kg)+low dose anti-Activin A (1 mg/kg), anti-GDF8 (6 mg/kg)+mid dose anti-Activin A (3 mg/kg), and anti-GDF8 (6 mg/kg)+high dose anti-Activin A (10 mg/kg) groups. At 8 weeks after single I.V. dose, a significant increase in thigh muscle volume % change from placebo was exhibited in anti-GDF8 (6 mg/kg)+mid dose anti-Activin A (3 mg/kg), and anti-GDF8 (6 mg/kg)+high dose anti-Activin A (10 mg/kg) groups. (*nominal p<0.05, **nominal p<0.001). N values for each group are shown in FIG. 2A.

FIG. 4 show line graphs depicting individual data for percent change (from baseline) in thigh muscle volume by MRI at weeks 0, 4, and 8 after a single I.V. dose of anti-Activin A antibody REGN2477 and/or anti-GDF8 antibody REGN1033 in healthy post-menopausal women as % change from baseline in placebo, anti-GDF8 (6 mg/kg), high dose anti-Activin A (10 mg/kg), anti-GDF8 (6 mg/kg)+low dose anti-Activin A (1 mg/kg), anti-GDF8 (6 mg/kg)+mid dose anti-Activin A (3 mg/kg), and anti-GDF8 (6 mg/kg)+high dose anti-Activin A (10 mg/kg) groups for multiple individuals. Increases in thigh muscle volume were consistently observed in individual subjects following treatment with REGN2477+REGN1033 in combination. Within each treatment group, different lines indicate different individuals.

FIG. 5 shows a line graph depicting appendicular lean (body) mass (i.e., sum of lean tissue in the arms and legs) at weeks 0, 4, and 8 after a single I.V. dose of anti-Activin A antibody REGN2477 and/or anti-GDF8 antibody REGN1033 in healthy post-menopausal women as % percent change from baseline LS (least-squares) mean (SE) in six groups. N values for each group are shown in FIG. 2A. After 4 weeks and 8 weeks, each of the three combination dose groups, including anti-GDF8 (6 mg/kg)+low dose anti-Activin A (1 mg/kg), anti-GDF8 (6 mg/kg)+mid dose anti-Activin A (3 mg/kg), and anti-GDF8 (6 mg/kg)+high dose anti-Activin A (10 mg/kg) groups, exhibited significantly increased % change LS mean difference in appendicular lean mass compared to placebo (*nominal p<0.05, **nominal p<0.001). N values for each group are shown in FIG. 2A.

FIG. 6 shows a line graph depicting mean (SE=standard errors) total fat mass percent (as measured by DXA) change at weeks 0, 4, and 8 after a single I.V. dose of anti-Activin A antibody REGN2477 and/or anti-GDF8 antibody REGN1033 in healthy post-menopausal women in placebo, anti-GDF8 (6 mg/kg), high dose anti-Activin A (10 mg/kg), anti-GDF8 (6 mg/kg)+low dose anti-Activin A (1 mg/kg), anti-GDF8 (6 mg/kg)+mid dose anti-Activin A (3 mg/kg), and anti-GDF8 (6 mg/kg)+high dose anti-Activin A (10 mg/kg) groups. N values for each group are shown in FIG. 2A. The high dose group, (anti-GDF8 (6 mg/kg)+high dose anti-Activin A (10 mg/kg), exhibited a significant reduction in total fat mass as percent change LS Mean difference compared to placebo at week 4 and week 8 (*nominal p<0.05). Blockade of both Activin A and GDF8 led to reductions in total fat mass, as assessed by DXA.

FIG. 7 shows a line graph depicting mean (SE) percent change in android fat mass at weeks 0, 4, and 8 after a single I.V. dose of anti-Activin A antibody REGN2477 and/or anti-GDF8 antibody REGN1033 in healthy post-menopausal women in six groups including placebo, anti-GDF8 (6 mg/kg), high dose (10 mg/kg) anti-Activin A, anti-GDF8 (6 mg/kg)+low dose (1 mg/kg) anti-Activin A, anti-GDF8 (6 mg/kg)+mid dose (3 mg/kg) anti-Activin A, and anti-GDF8 (6 mg/kg)+high dose (10 mg/kg) anti-Activin A groups. The high dose REGN1033+REGN2477 group exhibited significantly reduced percent change LS Mean difference in android fat mass by DXA compared to placebo at week 4 and week 8 (*nominal p<0.05). N values for each group are shown in FIG. 2A. Blockade of both Activin A and GDF8 was also associated with decreases in android fat mass, as assessed by DXA.

FIG. 8 shows a line graph depicting LS mean percent change with SE in thigh muscle volume (excluding intramuscular adipose tissue and large vessels) at weeks 0, 4, and 8 after a single I.V. dose of anti-Activin A antibody REGN2477 and/or anti-GDF8 antibody REGN1033 in healthy post-menopausal women in six groups including placebo, anti-GDF8 (6 mg/kg), high dose (10 mg/kg) anti-Activin A, anti-GDF8 (6 mg/kg)+low dose (1 mg/kg) anti-Activin A, anti-GDF8 (6 mg/kg)+mid dose (3 mg/kg) anti-Activin A, and anti-GDF8 (6 mg/kg)+high dose (10 mg/kg) anti-Activin A groups. N values for each group are shown in FIG. 2A. Compared with placebo, REGN2477+REGN1033 medium and high groups exhibited significantly increased mean % change in thigh muscle volume at 4 weeks and 8 weeks. (*nominal p<0.05, **nominal p<0.001).

FIG. 9 shows a line graph depicting LS mean percent change with SE in total lean mass at weeks 0, 4, and 8 after a single I.V. dose of anti-Activin A antibody REGN2477 and/or anti-GDF8 antibody REGN1033 in healthy post-menopausal women for six groups including placebo, anti-GDF8 (6 mg/kg), high dose (10 mg/kg) anti-Activin A, anti-GDF8 (6 mg/kg)+low dose (1 mg/kg) anti-Activin A, anti-GDF8 (6 mg/kg)+mid dose (3 mg/kg) anti-Activin A, and anti-GDF8 (6 mg/kg)+high dose (10 mg/kg) anti-Activin A groups. N values for each group are shown in FIG. 2A.

Compared with placebo, REGN2477+REGN1033 medium and high dose groups exhibited significantly increased % change in total lean mass compared to placebo at 4 weeks and 8 weeks. (*nominal p<0.05).

FIG. 10 shows a line graph depicting LS mean percent change with SE in appendicular lean body mass (calculated via aLBM equation), in kg, at weeks 0, 4, and 8 after a single I.V. dose of anti-Activin A antibody REGN2477 and/or anti-GDF8 antibody REGN1033 in healthy post-menopausal women in six groups including placebo, anti-GDF8, high dose (10 mg/kg) anti-Activin A, anti-GDF8+low dose (1 mg/kg) anti-Activin A, anti-GDF8+mid dose (3 mg/kg) anti-Activin A, and anti-GDF8+high dose (10 mg/kg) anti-Activin A groups. N values for each group are shown in FIG. 2A. REGN2477+REGN1033 treatment resulted in significantly increased % change in appendicular lean body mass calculated by aLBM equation) (kg) at each low, medium and high dose groups at 4 weeks and 8 weeks compared with placebo (*p<0.05, **p<0.001).

FIG. 11 shows a line graph depicting LS mean percent change with SE in total fat mass, in kg, at weeks 0, 4, and 8 after a single I.V. dose of anti-Activin A antibody REGN2477 and/or anti-GDF8 antibody REGN1033 in healthy post-menopausal women in six groups including placebo, anti-GDF8 (6 mg/kg), high dose (10 mg/kg) anti-Activin A, anti-GDF8+low dose (1 mg/kg) anti-Activin A, anti-GDF8 (6 mg/kg)+mid dose (3 mg/kg) anti-Activin A, and anti-GDF8 (6 mg/kg)+high dose (10 mg/kg) anti-Activin A groups. N values for each group are shown in FIG. 2A. The high dose combination REGN2477+REGN1033 treatment group exhibited significantly decreased % total fat mass: −3.92% (high dose group) compared with placebo −0.65% at 8 weeks. (*nominal p<0.05).

FIG. 12 shows a line graph depicting LS mean percent change with SE in thigh muscle volume, in cm³, (including intramuscular adipose tissue and large vessels) at weeks 0, 4, and 8 after a single I.V. dose of anti-Activin A antibody REGN2477 and/or anti-GDF8 antibody REGN1033 in healthy post-menopausal women in six groups including placebo, anti-GDF8, high dose (10 mg/kg) anti-Activin A, anti-GDF8+low dose (1 mg/kg) anti-Activin A, anti-GDF8+mid dose (3 mg/kg) anti-Activin A, and anti-GDF8+high dose (10mg/kg) anti-Activin A groups. N values for each group are shown in FIG. 2A. The high and medium dose REGN2477+REGN1033 treatment groups exhibited significantly increased % change in thigh muscle volume, including intramuscular adipose tissue and large vessels in medium and high treatment groups at 4 weeks and 8 weeks compared to placebo (*nominal p<0.05, **nominal p<0.001).

FIG. 13 shows a line graph depicting LS mean percent change with SE in appendicular lean mass (sum of lean mass of arms and legs), in kg, at weeks 0, 4, and 8 after a single I.V. dose of anti-Activin A antibody REGN2477 and/or anti-GDF8 antibody REGN1033 in healthy post-menopausal women in six groups including placebo, anti-GDF8, high dose (10 mg/kg) anti-Activin A, anti-GDF8+low dose (1 mg/kg) anti-Activin A, anti-GDF8+mid dose (3 mg/kg) anti-Activin A, and anti-GDF8+high dose (10 mg/kg) anti-Activin A groups. N values for each group are shown in FIG. 2A. Appendicular lean mass was significantly increased as mean percent change from placebo in each REGN2477+REGN1033 low, medium and high treatment groups at 4 and 8 weeks (*nominal p<0.05, *nominal* p<0.001).

FIG. 14 shows a line graph depicting LS mean percent change with SE in android fat mass, in kg, at weeks 0, 4, and 8 after a single I.V. dose of anti-Activin A antibody REGN2477 and/or anti-GDF8 antibody REGN1033 in healthy post-menopausal women in six groups including placebo, anti-GDF8, high dose (10 mg/kg) anti-Activin A, anti-GDF8+low dose (1 mg/kg) anti-Activin A, anti-GDF8+mid dose (3 mg/kg) anti-Activin A, and anti-GDF8+high dose (10 mg/kg) anti-Activin A groups. N values for each group are shown in FIG. 2A. The high dose REGN1033+REGN2477 group exhibited significantly reduced % change in android fat mass by DXA compared to placebo at week 4 and week 8 (*nominal p<0.05).

FIG. 15 shows a line graph depicting LS mean percent change with SE in thigh intramuscular adipose tissue volume, in cm³, at weeks 0, 4, and 8 after a single I.V. dose of anti-Activin A antibody REGN2477 and/or anti-GDF8 antibody REGN1033 in healthy post-menopausal women in six groups including placebo, anti-GDF8, high dose (10 mg/kg) anti-Activin A, anti-GDF8+low dose (1 mg/kg) anti-Activin A, anti-GDF8+mid dose (3 mg/kg) anti-Activin A, and anti-GDF8+high dose (10 mg/kg) anti-Activin A groups. N values for each group are shown in FIG. 2A. The high dose REGN1033+REGN2477 group exhibited increased thigh intramuscular adipose tissue volume as mean % change compared to placebo at 8 weeks. (*nominal p<0.05).

FIG. 16 shows a line graph depicting LS mean percent change with SE in sum of intramuscular and perimuscular adipose tissue (IMAT) at weeks 0, 4, and 8 after a single I.V. dose of anti-Activin A antibody REGN2477 and/or anti-GDF8 antibody REGN1033 in healthy post-menopausal women in six groups including placebo, anti-GDF8, high dose (10 mg/kg) anti-Activin A, anti-GDF8+low dose (1 mg/kg) anti-Activin A, anti-GDF8+mid dose (3 mg/kg) anti-Activin A, and anti-GDF8+high dose (10 mg/kg) anti-Activin A groups. N values for each group are shown in FIG. 2A. The combination REGN1033+REGN2477 treatment groups were not significantly different than placebo in LS mean percent change with SE in sum of intramuscular and perimuscular adipose tissue (IMAT) at weeks 4 and 8.

FIG. 17 shows a line graph depicting LS mean percent change with SE in subcutaneous adipose tissue volume, in cm³, at weeks 0, 4, and 8 after a single I.V. dose of anti-Activin A antibody REGN2477 and/or anti-GDF8 antibody REGN1033 in healthy post-menopausal women in six groups including placebo, anti-GDF8, high dose (10 mg/kg) anti-Activin A, anti-GDF8+low dose (1 mg/kg) anti-Activin A, anti-GDF8+mid dose (3 mg/kg) anti-Activin A, and anti-GDF8+high dose (10 mg/kg) anti-Activin A groups. N values for each group are shown in FIG. 2A. The REGN1033+REGN2477 low and medium treatment groups exhibited significantly decreased % change in subcutaneous adipose tissue volume compared to placebo at 8 weeks (*nominal p<0.05).

### DETAILED DESCRIPTION

Before the present invention is described, it is to be understood that this invention is not limited to particular compositions, methods, and experimental conditions described, as such compositions, methods, and conditions may vary. It is also to be understood that the terminology used herein is for the purpose of describing particular embodiments only, and is not intended to be limiting, since the scope of the present invention will be limited only by the appended claims. Unless defined otherwise, all technical and scientific terms used herein have the same meaning as commonly understood by one of ordinary skill in the art to which this invention belongs.

As used in this specification and the appended claims, the singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice of the present invention, the preferred methods and materials are now described. All publications mentioned herein are incorporated herein by reference in their entirety.

### Antibodies and Antigen-Binding Fragments of Antibodies

The term “antibody”, as used herein, is intended to refer to immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the invention, the FRs of the antibodies (or antigen-binding portion thereof) may be identical to the human germline sequences, or may be naturally or artificially modified. An amino acid consensus sequence may be defined based on a side-by-side analysis of two or more CDRs.

Antibodies may be referred to herein according to the following nomenclature: Fc prefix (e.g. “H1M,” “H2aM,” “H4H”), followed by a numerical identifier (e.g. “10446”), followed by a “P,” “P2” or “N” suffix. Thus, according to this nomenclature, an antibody may be referred to herein as, e.g., “H4H10446P2”. The H1M, H2M and H4H prefixes on the antibody designations used herein indicate the particular Fc region isotype of the antibody. For example, an “H2aM” antibody has a mouse IgG2a Fc, whereas an “H4H” antibody has a human IgG4 Fc. As will be appreciated by a person of ordinary skill in the art, an antibody having a particular Fc isotype can be converted to an antibody with a different Fc isotype (e.g., an antibody with a mouse IgG2a Fc can be converted to an antibody with a human IgG4, etc.), but in any event, the variable domains (including the CDRs) will remain the same, and the binding properties are expected to be identical or substantially similar regardless of the nature of the Fc domain.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR that is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody used in the present invention include: (i) V_{H}-C_{H}1;(ii) V_{H}-C_{H}2; (iii) V_{H}-C_{H}3; (iv) V_{H}-C_{H}1-C_{H}2; (v) V_{H}-C_{H}1-C_{H}2-C_{H}3; (Vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (Xi) V_{L}-C_{H}1-C_{H}2; (Xii) V_{L}-C_{H}1-C_{H}2-C_{H}3; (Xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody used in the present invention may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

Full antibody molecules and antigen-binding fragments may be monospecific or multispecific (e.g., bispecific). A multispecific antibody or antigen-binding fragment of an antibody will typically comprise at least two different variable domains, wherein each variable domain is capable of specifically binding to a separate antigen or to a different epitope on the same antigen. Any multispecific antibody format may be adapted for use in the context of an antibody or antigen-binding fragment described herein using routine techniques available in the art.

The antibodies used in the compositions and methods of the invention may function through complement-dependent cytotoxicity (CDC) or antibody-dependent cell-mediated cytotoxicity (ADCC). “Complement-dependent cytotoxicity” (CDC) refers to lysis of antigen-expressing cells by an antibody of the invention in the presence of complement. “Antibody-dependent cell-mediated cytotoxicity” (ADCC) refers to a cell-mediated reaction in which nonspecific cytotoxic cells that express Fc receptors (FcRs) (e.g., Natural Killer (NK) cells, neutrophils, and macrophages) recognize bound antibody on a target cell and thereby lead to lysis of the target cell. CDC and ADCC can be measured using assays that are well known and available in the art. (See, e.g., U.S. Pat. Nos. 5,500,362 and 5,821,337, and Clynes et al., Proc. Natl. Acad. Sci. (USA) 95:652-656 (1998)).

The antibodies described herein may comprise or consist of human antibodies and/or recombinant human antibodies, or fragments thereof. The term “human antibody”, as used herein, includes antibodies having variable and constant regions derived from human germline immunoglobulin sequences. Human antibodies may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

The antibody molecules described herein may comprise or consist of recombinant human antibodies or antigen-binding fragments thereof. The term “recombinant human antibody”, as used herein, is intended to include all human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo. Recombinant antibodies may be used in an additional embodiment of the compositions and methods of the invention.

An “isolated antibody,” as used herein, means an antibody that has been identified and separated and/or recovered from at least one component of its natural environment. For example, an antibody that has been separated or removed from at least one component of an organism, tissue or cell in which the antibody naturally exists or is naturally produced is an “isolated antibody” for purposes of the invention. An isolated antibody also includes an antibody in situ within a recombinant cell, as well as an antibody that has been subjected to at least one purification or isolation step. According to certain embodiments, an isolated antibody may be substantially free of other cellular material and/or chemicals. Isolated antibodies may be used in an additional embodiment of the compositions and methods of the invention.

A protein or polypeptide is “substantially pure,” “substantially homogeneous” or “substantially purified” when at least about 60 to 75% of a sample exhibits a single species of polypeptide. The polypeptide or protein may be monomeric or multimeric. A substantially pure polypeptide or protein will typically comprise about 50%, 60, 70%, 80% or 90% w/w of a protein sample, usually about 95%, and preferably over 99% pure. Protein purity or homogeneity may be indicated by a number of means well known in the art, such as polyacrylamide gel electrophoresis of a protein sample, followed by visualizing a single polypeptide band upon staining the gel with a stain well known in the art. For certain purposes, higher resolution may be provided by using HPLC or other means well known in the art for purification.

The term “polypeptide analog or variant” as used herein refers to a polypeptide that is comprised of a segment of at least 25 amino acids that has substantial identity to a portion of an amino acid sequence and that has at least one of the following properties: (1) specific binding to GDF8 under suitable binding conditions, or (2) ability to block the biological activity of GDF8. Typically, polypeptide analogs or variants comprise a conservative amino acid substitution (or insertion or deletion) with respect to the naturally occurring sequence. Analogs typically are at least 20 amino acids long, at least 50, 60, 70, 80, 90, 100, 150 or 200 amino acids long or longer, and can often be as long as a full-length naturally-occurring polypeptide.

Preferred amino acid substitutions are those which: (1) reduce susceptibility to proteolysis, (2) reduce susceptibility to oxidation, (3) alter binding affinity for forming protein complexes, (4) alter binding affinities, and (4) confer or modify other physicochemical or functional properties of such analogs. Analogs can include various mutations of a sequence other than the naturally-occurring peptide sequence. For example, single or multiple amino acid substitutions (preferably conservative amino acid substitutions) may be made in the naturally occurring sequence (preferably in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton 1984 W. H. Freeman and Company, New York; Introduction to Protein Structure (Branden & Tooze, eds., 1991, Garland Publishing, NY); and Thornton et at. 1991 Nature 354:105, which are each incorporated herein by reference.

Non-peptide analogs are commonly used in the pharmaceutical industry as drugs with properties analogous to those of the template peptide. These types of non-peptide compound are termed “peptide mimetics” or “peptidomimetics” (see, for example, Fauchere (1986) J. Adv. Drug Res. 15:29; and Evans et al. (1987) J. Med. Chem. 30:1229, which are incorporated herein by reference. Systematic substitution of one or more amino acids of a consensus sequence with a D-amino acid of the same type (e.g., D-lysine in place of L-lysine) may also be used to generate more stable peptides. In addition, constrained peptides comprising a consensus sequence or a substantially identical consensus sequence variation may be generated by methods known in the art (Rizo et al. (1992) Ann. Rev. Biochem. 61:387, incorporated herein by reference), for example, by adding internal cysteine residues capable of forming intramolecular disulfide bridges which cyclize the peptide.

As applied to polypeptides, the term “substantial identity” or “substantially identical” means that two peptide sequences, when optimally aligned, such as by the programs GAP or BESTFIT using default gap weights, share at least about 80% sequence identity, at least about 90%, at least about 95%, at least about 98% or at least about 99% sequence identity. Preferably, residue positions that are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24:307-331, herein incorporated by reference. Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; and 6) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256:1443-45, herein incorporated by reference. A “moderately conservative” replacement is any change having a nonnegative value in the PAM250 log-likelihood matrix.

Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson (2000), supra). Another preferred algorithm when comparing a sequence of the invention to a database containing a large number of sequences from different organisms is the computer program BLAST, especially blastp or tblastn, using default parameters. See, e.g., Altschul et al. (1990) J. Mol. Biol. 215:403-410 and Altschul et al. (1997) Nucleic Acids Res. 25:3389 402, each of which is herein incorporated by reference.

The length of polypeptide sequences compared for homology will generally be at least about 16 amino acid residues, at least about 20 residues, at least about 24 residues, at least about 28 residues, or at least about 35 residues. When searching a database containing sequences from a large number of different organisms, it is preferable to compare amino acid sequences.

In some embodiments, the invention relates to a method for altering the body composition of a subject comprising administering a first composition comprising an effective amount of a GDF8 inhibitor and a second composition comprising an effective amount of an Activin A inhibitor to the subject.

In some embodiments, the invention relates to a method for inducing a reduction in fat mass in a subject comprising administering a first composition comprising an effective amount of a GDF8 inhibitor and a second composition comprising an effective amount of an Activin A inhibitor to the subject.

In some embodiments, the invention relates to a method for inducing an increase in muscle mass in a subject, the method comprising administering to a subject in need thereof a first composition comprising an effective amount of a GDF8 inhibitor and a second composition comprising an effective amount of an Activin A inhibitor.

In some embodiments, the invention relates to a method for altering the body composition of a subject comprising administering a composition comprising an effective amount of a GDF8 inhibitor and an effective amount of an Activin A inhibitor to the subject.

In some embodiments, the invention relates to a method for inducing a reduction in fat mass in a subject comprising administering a composition comprising an effective amount of a GDF8 inhibitor and an effective amount of an Activin A inhibitor to the subject.

In some embodiments, the invention relates to a method for inducing an increase in muscle mass in a subject, the method comprising administering to a subject in need thereof a composition comprising an effective amount of a GDF8 inhibitor and an effective amount of an Activin A inhibitor.

In some embodiments, the subject is a human subject. The human subject may be an adult human subject. The subject may be a male or female subject. The subject may be a healthy subject. The subject may be suffering from unwanted fat mass. The subject may be suffering from, or at risk of suffering from, a disease and disorder characterized by increased fat mass, and/or decreased muscle volume or decreased lean body mass. The subject may be a post-menopausal female subject. The subject may be a male subject or female subject 40 years of age or older, 50 years of age or older, 60 years of age or older, or 70 years of age or older.

In some embodiments, the invention relates to a method comprising administering a composition comprising a GDF8 inhibitor and a composition comprising an Activin A inhibitor to a subject in need thereof for treating or preventing diseases and disorders characterized by increased fat mass, and/or decreased muscle volume or decreased lean body mass.

In some embodiments according to the invention, the subject has at least one disease or disorder that may be associated with increased fat mass. In some embodiments, the disease or disorder may be selected from the group consisting of obesity, metabolic syndromes, nutritional disorders, high cholesterol, dyslipidemia, cardiovascular disease, cellulitis, cancer (including of the colon, esophagus, kidney, pancreas, gallbladder, breast, or endometrium), polycystic ovarian syndrome, gout, gallbladder disease, sleep apnea, respiratory disorder, asthma, osteoarthritis, cataract, congestive heart failure, enlarged heart, high blood pressure/hypertension, pulmonary embolism, lymphedema, gastro-esophageal reflux disease, hernia, chronic renal failure, urinary incontinence, connective tissue diseases, and fatty-liver disease. In another embodiment, the disease or disorder may be sarcopenia.

### GDF8 Inhibitors

The present invention includes methods for altering body composition, inducing a reduction in fat mass, and increasing lean mass in a subject, and methods for treating a disease or disorder characterized by increased fat mass in a subject, comprising administering a composition comprising an effective amount of a GDF8 inhibitor to the subject.

The term “GDF8” (also referred to as “growth and differentiation factor-8” and “myostatin”) means the protein having the amino acid sequence of SEQ ID NO:340 (mature protein). According to the present invention, GDF8-specific binding proteins specifically bind GDF8 but do not bind other ActRIIB ligands such as GDF3, BMP2, BMP4, BMP7, BMP9, BMP10, GDF11, Activin A, Activin B, Activin AB, Nodal, etc.

As used herein, a “GDF8 inhibitor” is any agent that binds to or interacts with human GDF8 and interferes with or inhibits the normal biological function of GDF8 in vitro or in vivo. Non-limiting examples of categories of GDF8 inhibitors include small molecule GDF8 antagonists, nucleic acid-based inhibitors of GDF8 expression or activity (e.g., siRNA or antisense), peptide-based molecules that specifically interact with GDF8 (e.g., peptibodies), receptor molecules that specifically interact with GDF8, GDF8-binding scaffold molecules, proteins comprising a ligand-binding portion of a receptor that specifically binds GDF8, and anti-GDF8 aptamers or portions thereof. In a preferred embodiment, a GDF8 inhibitor that can be used in the context of the present invention is an anti-GDF8 antibody or antigen-binding fragment thereof that specifically binds human GDF8. Anti-GDF8 antibodies include neutralizing and/or blocking antibodies. The inhibition caused by anti-GDF8 neutralizing and/or blocking antibodies need not be complete, as long as it is detectable using appropriate assays.

As used herein, the expression “anti-GDF8 antibody” also includes multispecific antigen-binding molecules (e.g., bispecific antibodies), wherein at least one binding domain (e.g., “binding arm”) of the multispecific antigen-binding molecule specifically binds GDF8.

Exemplary anti-GDF8 antibodies that can be used in the compositions and methods of the invention include, e.g., the fully-human anti-GDF8 antibody H4H1657N2, also known as REGN1033, (e.g., an anti-GDF8 antibody comprising the heavy and light chain variable regions having amino acid sequences SEQ ID NO: 360 and SEQ ID NO: 368, respectively, as set forth in U.S. Pat. No. 8,840,894). Other GDF8 antagonists that can be used in the compositions and methods of the invention include anti-GDF8 antibodies (e.g., the antibody designated 2_112_1, e.g., having ATCC deposit designation PTA-6574, or e.g., 2_112_K, e.g., having HCVR/LCVR amino acid sequences SEQ ID NOs: 620 and 621) as set forth in US 2006/0263354 and U.S. Pat. No. 7,807,159; anti-GDF8 antibodies (e.g., 12A5-5, e.g., having HCVR/LCVR amino acid sequences of SEQ ID NO: 622 and 623) as set forth in U.S. Pat. No. 8,999,343 and US Publication No. 2013/0209489; anti-GDF8 antibodies (e.g., 10B3H8L5, e.g., having HCVR/LCVR amino acid sequences of SEQ ID NO:624 and 625, and 10B3H8L5-Fc-disabled) as set forth in US Publication No. 2013/0142788; anti-GDF8 antibodies (e.g., stamulumab/MYO-29, e.g., having HCVR/LCVR amino acid sequences of SEQ ID NOs: 626 and 627) as set forth in U.S. Pat. Nos. 8,940,874 and 7,261,893; anti-GDF8 antibodies (e.g., RK22/PF-0625616, e.g., having HCVR/LCVR amino acid sequences of SEQ ID NO: 628 and 629) as set forth in U.S. Pat. No. 8,415,459; anti-GDF8 antibodies (e.g., JA-16, e.g., having CDRs of HCVR amino acid sequence of SEQ ID NO: 630) as set forth in U.S. Pat. No. 7,731,961; anti-GDF8 antibodies (e.g., RK35, e.g., having HCVR/LCVR amino acid sequences of SEQ ID NO: 631 and 632) as set forth in U.S. Pat. No. 8,496,934 or 7,888,486, anti-GDF8 antibodies (e.g., OGD1.0.0, e.g., having HCVR/LCVR amino acid sequences of SEQ ID NO: 633 and 634) as set forth in U.S. Pat. No. 8,992,913; anti-GDF8 Fab molecules as set forth in European Patent No. 1 773 041 B1, and anti-GDF8 antibodies (e.g., C12, e.g., having HCVR/LCVR amino acid sequences of SEQ ID NOs: 635 and 636, C12-N93H, and/or 510C2 having HCVR/LCVR amino acid sequences of SEQ ID NOs: 637 and 638) as set forth in, e.g., U.S. Pat. Nos. 7,635,760 and 8,063,188, anti-GDF8 antibodies (e.g. 41C1E4/landogrozumab/LY2495655, e.g., having HCVR/LCVR amino acid sequences of SEQ ID NO: 639 and 640) as set forth in U.S. Pat. No. 7,632,499. In some embodiments, the anti-GDF8 antibody may have the full length heavy chain and full length light chain amino acid sequences of landogrozumab, e.g., SEQ ID Nos 641 and 642, respectively. In some embodiments, the anti-GDF8 antibody may comprise three heavy chain CDRs (HCDRs) and three light chain CDRs (LCDRs) of landogrozumab, for example, by Chothia definition, according to SEQ ID Nos: 643/644/645/646/647/648, respectively.

In one embodiment, an anti-GDF8 antibody or antigen-binding fragment thereof comprises a heavy chain variable region (HCVR) having an amino acid sequence selected from the group consisting of SEQ ID NO:2, 18, 34, 50, 66, 82, 98, 114, 130, 146, 162, 178, 194, 210, 226, 242, 258, 274, 290, 306, 360, and 376, or a substantially similar sequence thereof having at least 90%, at least 95%, at least 98% or at least 99% sequence identity.

In another embodiment, an anti-GDF8 antibody or antigen-binding fragment thereof comprises a light chain variable region (LCVR) amino acid sequence selected from the group consisting of SEQ ID NO: 10, 26, 42, 58, 74, 90, 106, 122, 138, 154, 170, 186, 202, 218, 234, 250, 266, 282, 298, 314, 322, 368, and 384, or a substantially similar sequence thereof having at least 90%, at least 95%, at least 98% or at least 99% sequence identity.

In another embodiment, an anti-GDF8 antibody or antigen-binding fragment thereof comprises a HCVR amino acid sequence and a LCVR amino acid sequence, wherein the HCVR/LCVR sequence pair is selected from the group consisting of SEQ ID NO:2/10, 18/26, 34/42, 50/58, 66/74, 82/90, 98/106, 114/122, 130/138, 146/154, 162/170, 178/186, 194/202, 210/218, 226/234, 242/250, 258/266, 274/282, 290/298, 306/314, 114/322, 360/368, and 376/384.

In another embodiment, an anti-GDF8 antibody or antigen-binding fragment thereof comprises a HCVR amino acid sequence and a LCVR amino acid sequence, wherein the HCVR/LCVR sequence pair is selected from the group consisting of (HCVR/LCVR): 21-E5 (SEQ ID NO:34/42); 21-B9 (SEQ ID NO:18/26); 21-E9 (SEQ ID NO:98/106); 21-A2 (SEQ ID NO: 2/10); 22-D3 (SEQ ID NO:50/58); 22-E6 (SEQ ID NO:66/74); 22-G10 (SEQ ID NO: 82/90); 1A2 (SEQ ID NO:226/234); 20B12 (SEQ ID NO:274/282); 58C8 (SEQ ID NO: 242/250); 19F2 (SEQ ID NO:258/266); 8D12-1 (SEQ ID NO: 114/122); 4E3-7 (SEQ ID NO: 194/202); 9B11-12 (SEQ ID NO:162/170); 4B9 (SEQ ID NO:226/234); 1H4-5 (SEQ ID NO: 210/218); 9B4-3 (SEQ ID NO: 178/186); 3E2-1 (SEQ ID NO:290/298); 4G3-25 (SEQ ID NO: 306/314); 4B6-6 (SEQ ID NO: 130/138); H4H1657N2 (SEQ ID NO:360/368); H4H1669P (SEQ ID NO:376/384).

In another embodiment, an anti-GDF8 antibody or antigen-binding fragment thereof comprises a heavy chain complementarity determining region 3 (HCDR3) domain and a light chain CDR3 (LCDR3) domain, wherein the HCDR3 domain has an amino acid sequence selected from the group consisting of SEQ ID NO:8, 24, 40, 56, 72, 88, 104, 120, 136, 152, 168, 184, 200, 216, 232, 248, 264, 280, 296, 312, 366, and 382, or a substantially similar sequence thereof having at least 90%, at least 95%, at least 98% or at least 99% sequence identity, and the LCDR3 domain has an amino acid sequence selected from the group consisting of SEQ ID NO: 16, 32, 48, 64, 80, 96, 112, 128, 144, 160, 176, 192, 208, 224, 240, 256, 272, 288, 304, 320, 328, 374, and 390, or a substantially similar sequence thereof having at least 90%, at least 95%, at least 98% or at least 99% sequence identity. In another embodiment, an anti-GDF8 antibody or antigen-binding fragment thereof comprises an HCDR3/LCDR3 amino acid sequence pair selected from the group consisting of SEQ ID NO:8/16, 24/32, 40/48, 56/64, 72/80, 88/96, 104/112, 120/128,136/144, 152/160, 168/176, 184/192, 200/208, 216/224, 232/240, 248/256, 264/272,280/288, 296/304, 312/320, 120/328, 366/374, and 382/390.

In another embodiment, an anti-GDF8 antibody or antigen-binding fragment thereof comprises heavy chain CDR1 (HCDR1) and CDR2 (HCDR2) domains and light chain CDR1 (LCDR1) and CDR2 (LCDR2) domains, wherein the HCDR1 domain has an amino acid sequence selected from the group consisting of SEQ ID NO:4, 20, 36, 52, 68, 84, 100, 116, 132, 148, 164, 180, 196, 212, 228, 244, 260, 276, 292, 308, 362, and 378, or a substantially similar sequence thereof having at least 90%, at least 95%, at least 98% or at least 99% sequence identity; the HCDR2 domain has an amino acid sequence selected from the group consisting of SEQ ID NO:6, 22, 38, 54, 70, 86, 102, 118, 134, 150, 166, 182, 198, 214, 230, 246, 262, 278, 294, 310, 364, and 380, or a substantially similar sequence thereof having at least 90%, at least 95%, at least 98% or at least 99% sequence identity; the LCDR1 domain has an amino acid sequence selected from the group consisting of SEQ ID NO: 12, 28, 44, 60, 76, 92, 108, 124, 140, 156, 172, 188, 204, 220, 236, 252, 268, 284, 300, 316, 324, 370, and 386, or a substantially similar sequence thereof having at least 90%, at least 95%, at least 98% or at least 99% sequence identity and the LCDR2 domain has an amino acid sequence selected from the group consisting of GVS, AAS, AAS, AAS, AAS, AAS, AAS, RMS, VAS, AAS, GAS, ATS, GAS, GAS, GAS, GAS, LGS, GAS, AAS, LGS, KIS, TTS, and AAS, or a substantially similar sequence thereof having at least 90%, at least 95%, at least 98% or at least 99% sequence identity. In another embodiment, the HCDR1, HCDR2 and HCDR3 domains have respective amino acid sequence combinations selected from the group consisting of SEQ ID NO:36/38/40, 116/118/120, 228/230/232, 362/364/366, and 378/380/382; and the LCDR1, LCDR2 and LCDR3 domains have respective amino acid sequence combinations selected from the group consisting of SEQ ID NO:44/AAS/48, 124/RMS/128, 236/GAS/240, 370/TTS/374, and 386/AAS/390.

In yet another embodiment, the heavy and light chain CDR domains of the anti-GDF8 antibody or antigen-binding fragment thereof (HCDR1/HCDR2/HCDR3/LCDR1/LCDR2/LCDR3) have amino acid sequence combinations selected from the group consisting of SEQ ID NO: 36/38/40/44/AAS/48 (e.g., 21-E5), 116/118/120/124/RMS/128 (e.g., 8D12), 228/230/232/236/GAS/240 (e.g., 1A2), 362/364/366/370/TTS/374 (e.g., H4H1657N2), and 378/380/382/386/AAS/390 (e.g., H4H1669P).

In another embodiment, an anti-GDF8 antibody or antigen-binding fragment thereof comprises heavy and light chain CDR domains contained within heavy and light chain variable region (HCVR/LCVR) amino acid sequence pairs selected from the group consisting of SEQ ID NO: 2/10, 18/26, 34/42, 50/58, 66/74, 82/90, 98/106, 114/122, 130/138, 146/154, 162/170, 178/186, 194/202, 210/218, 226/234, 242/250, 258/266, 274/282, 290/298, 306/314, 114/322, 360/368, and 376/384.

Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani et al., J. Mol. Biol. 273:927-948 (1997); and Martin et al., Proc. Natl. Acad. Sci. USA 86:9268-9272 (1989). Public databases are also available for identifying CDR sequences within an antibody.

In one embodiment, an anti-GDF8 antibody or antigen-binding fragment thereof comprises a HCVR having an amino acid sequence encoded by a nucleic acid sequence selected from the group consisting of SEQ ID NO:1, 17, 33, 49, 65, 81, 97, 113, 129, 145, 161, 177, 193, 209, 225, 241, 257, 273, 289, 305, 359, and 375, or a substantially similar sequence thereof having at least 90%, at least 95%, at least 98% or at least 99% sequence identity.

In another embodiment, an anti-GDF8 antibody or antigen-binding fragment thereof comprises a LCVR having an amino acid sequence encoded by a nucleic acid sequence selected from the group consisting of SEQ ID NO:9, 25, 41, 57, 73, 89, 105, 121, 137, 153, 169, 185, 201, 217, 233, 249, 265, 281, 297, 313, 321, 367, and 383, or a substantially similar sequence thereof having at least 90%, at least 95%, at least 98% or at least 99% sequence identity.

In another embodiment, an anti-GDF8 antibody or antigen-binding fragment thereof comprises a HCVR/LCVR pair having amino acid sequences encoded by a nucleic acid molecule pair selected from the group consisting of SEQ ID NO: 1/9, 17/25, 33/41, 49/57, 65/73, 81/89, 97/105, 113/121, 129/137, 145/153, 161/169, 177/185, 193/201, 209/217, 225/233, 241/249, 257/265, 273/281, 289/297, 305/313, 113/321, 359/367, and 375/383.

In another embodiment, an anti-GDF8 antibody or antigen-binding fragment thereof comprises a HCDR3 domain having an amino acid sequence encoded by a nucleic acid sequence selected from the group consisting of SEQ ID NO:7, 23, 39, 55, 71, 87, 103, 119, 135, 151, 167, 183, 199, 215, 231, 247, 263, 279, 295, 311, 365, and 381, or a substantially similar sequence thereof having at least 90%, at least 95%, at least 98% or at least 99% sequence identity, and a LCDR3 domain having an amino acid sequence encoded by a nucleic acid sequence selected from the group consisting of SEQ ID NO: 15, 31, 47, 63, 79, 95, 111, 127, 143, 159, 175, 191, 207, 223, 239, 255, 271, 287, 303, 319, 327, 373, and 389, or a substantially similar sequence thereof having at least 90%, at least 95%, at least 98% or at least 99% sequence identity. In one embodiment, the HCDR3/LCDR3 domain pair has amino acid sequences encoded by a nucleic acid sequence pair selected from the group consisting of SEQ ID NO:7/15, 23/31, 39/47, 55/63, 71/79, 87/95, 103/111, 119/127, 135/143, 151/159, 167/175, 183/191, 199/207, 215/223, 231/239, 247/255, 263/271, 279/287, 295/303, 311/319, 119/327, 365/373, and 381/389.

In another embodiment, an anti-GDF8 antibody or antigen-binding fragment thereof comprises HCDR1 and HCDR2 domains, and LCDR1 and LCDR2 domains, wherein the HCDR1 domain has an amino acid sequence encoded by a nucleic acid sequence selected from the group consisting of SEQ ID NO:3, 19, 35, 51, 67, 83, 99, 115, 131, 147, 163, 179, 195, 211, 227, 243, 259, 275, 291, 307, 361, and 377, or a substantially similar sequence thereof having at least 90%, at least 95%, at least 98% or at least 99% sequence identity, the HCDR2 domain has an amino acid sequence encoded by a nucleic acid sequence selected from the group consisting of SEQ ID NO:5, 21, 37, 53, 69, 85, 101, 117, 133, 149, 165, 181, 197, 213, 229, 245, 261, 277, 293, 309, 363, and 379, or a substantially similar sequence thereof having at least 90%, at least 95%, at least 98% or at least 99% sequence identity, the LCDR1 domain has an amino acid sequence encoded by a nucleic acid sequence selected from the group consisting of SEQ ID NO: 11, 27, 43, 59, 75, 91, 107, 123, 139, 155, 171, 187, 203, 219, 235, 251, 267, 283, 299, 315, 323, 369, and 385, or a substantially similar sequence thereof having at least 90%, at least 95%, at least 98% or at least 99% sequence identity, and the LCDR2 domain has an amino acid sequence encoded by a nucleic acid sequence selected from the group consisting of ggtgtatcc, gctgcatcc, gctgcatcc, gctgcatcc, gctgcatcc, gctgcatcc, gctgcatcc, cggatgtcc, gttgcatcc, gctgcatcc, ggtgcatcc, gctacatcc, ggtgcatcc, ggtgcatcc, ggtgcatcc, ggtgcatcc, ttgggttct, ggtgcatcc, gctgcatcc, ttgggttct, aagatttct, actacatcc, and gctgcatcc.

In another embodiment, an anti-GDF8 antibody or antigen-binding fragment thereof comprises heavy and light chain CDR domains (HCDR1/HCDR2/HCDR3/LCDR1/LCDR2/LCDR3) having amino acid sequence combinations encoded by a nucleic acid sequence set of SEQ ID NO: 35/37/39/43/gctgcatcc/47, 115/117/119/123/cggatgtcc/127, 227/229/231/235/ggtgcatcc/239, 361/363/365/369/actacatcc/373, or 377/379/381/385/gctgcatcc/389.

In a preferred embodiment, the anti-GDF8 antibody or antigen-binding fragment thereof that specifically binds GDF8 comprises the HCDRs of a heavy chain variable region (HCVR) comprising SEQ ID NO:360 and the LCDRs of a light chain variable region (LCVR) comprising SEQ ID NO:368. In another embodiment, the anti-GDF8 antibody or antigen-binding fragment that specifically binds GDF8 comprises three HCDRs comprising SEQ ID NO: 362, SEQ ID NO:364, and SEQ ID NO:366 and three LCDRs comprising SEQ ID NO: 370, TTS, and SEQ ID NO:374.

In another embodiment, an anti-GDF8 antibody or antigen-binding fragment thereof is a fully human or humanized antibody or antibody fragment that binds GDF8 with an affinity (expressed as a dissociation constant, “K_{D}”) of about 1 nM or less, as measured by surface plasmon resonance assay (for example, BIACORE™). In certain embodiments, the antibody of the invention exhibits a K_{D} of about 700 pM or less; about 500 pM or less; about 320 pM or less; about 160 pM or less; about 100 pM or less; about 50 pM or less; about 10 pM or less; or about 5 pM or less.

In another embodiment, an anti-GDF8 antibody or antigen-binding fragment thereof is a fully human or humanized monoclonal antibody (mAb) that specifically binds and inhibits human GDF8 and exhibits an IC50 of less than or equal to about 10 nM; about 5 nM or less; about 3 nM or less; about 2 nM or less; about 1 nM or less; about 500 pM or less; or about 200 pM or less, as measured by GDF8 inducible luciferase assay.

In one embodiment, an anti-GDF8 antibody or antigen-binding fragment thereof has a modified glycosylation pattern. In some applications, modification to remove undesirable glycosylation sites may be useful, or an antibody lacking a fucose moiety present on the oligosaccharide chain, for example, to increase antibody dependent cellular cytotoxicity (ADCC) function (Shield et al. (2002) JBC 277:26733). In other applications, modification of a galactosylation can be made in order to modify complement dependent cytotoxicity (CDC).

In another embodiment, an anti-GDF8 antibody or antigen-binding fragment thereof competes for specific binding to GDF8 with another antibody comprising a

HCDR1/HCDR2/HCDR3/LCDR1/LCDR2/LCDR3 domain combination having amino acid sequences selected from the group consisting of SEQ ID NO:36/38/40/44/AAS/48, 116/118/120/124/RMS/128, 228/230/232/236/GAS/240, 362/364/366/370/TTS/374, or 378/380/382/386/AAS/390. In another embodiment, an anti-GDF8 antibody or antigen-binding fragment thereof competes for specific binding to GDF8 with another antibody comprising a HCVR/LCVR amino acid sequence pair of SEQ ID NO:2/10, 18/26, 34/42, 50/58, 66/74, 82/90, 98/106, 114/122, 130/138, 146/154, 162/170, 178/186, 194/202, 210/218, 226/234, 242/250, 258/266, 274/282, 290/298, 306/314, 114/322, 360/368, or 376/384.

In another embodiment, an anti-GDF8 antibody or antigen-binding fragment thereof recognizes the epitope on GDF8 that is recognized by another antibody comprising a HCDR1/HCDR2/HCDR3/LCDR1/LCDR2/LCDR3 domain combination having amino acid sequences selected from the group consisting of SEQ ID NO: 36/38/40/44/AAS/48, 116/118/120/124/RMS/128, 228/230/232/236/GAS/240, 362/364/366/370/TTS/374, or 378/380/382/386/AAS/390. In another embodiment, an anti-GDF8 antibody or antigen-binding fragment thereof recognizes the epitope on GDF8 that is recognized by another antibody comprising a HCVR/LCVR amino acid sequence pair of SEQ ID NO:2/10, 18/26, 34/42, 50/58, 66/74, 82/90, 98/106, 114/122, 130/138, 146/154, 162/170, 178/186, 194/202, 210/218, 226/234, 242/250, 258/266, 274/282, 290/298, 306/314, 114/322, 360/368, or 376/384.

### Activin A Inhibitors

The present invention includes methods for altering body composition, inducing a reduction in fat mass, and/or increasing lean mass in a subject, and methods for treating a disease or disorder characterized by increased fat mass in a subject comprising administering a composition comprising an effective amount of an Activin A inhibitor to the subject.

As used herein, an “Activin A inhibitor” is any agent that binds to or interacts with human Activin A and interferes with or inhibits the normal biological function of Activin A in vitro or in vivo. Non-limiting examples of categories of Activin A inhibitors include small molecule Activin A antagonists, nucleic acid-based inhibitors of Activin A expression or activity (e.g., siRNA or antisense), peptide-based molecules that specifically interact with Activin A (e.g., peptibodies), receptor molecules that specifically interact with Activin A, Activin A-binding scaffold molecules, proteins comprising a ligand-binding portion of a receptor that specifically binds Activin A, and anti-Activin A aptamers or portions thereof. In a preferred embodiment, an Activin A inhibitor that can be used in the context of the present invention is an anti-Activin A antibody or antigen-binding fragment thereof that specifically binds human Activin A. Anti-Activin A antibodies include neutralizing and/or blocking antibodies. The inhibition caused by anti-Activin A neutralizing and/or blocking antibodies need not be complete, as long as it is detectable using appropriate assays.

Activins are homo-and hetero-dimeric molecules comprising βA and/or βB subunits. The βA subunit has the amino acid sequence of SEQ ID NO:617 and the βB subunit has the amino acid sequence of SEQ ID NO: 619. Activin A is a homodimer of two βA subunits; Activin B is a homodimer of two βB subunits; and Activin AB is a heterodimer of one βA subunit and one βB subunit. An anti-Activin A antibody or antigen-binding fragment thereof specifically binds the βA subunit. Since the βA subunit is found in both Activin A and Activin AB molecules, an “anti-Activin A antibody or antigen-binding fragment thereof” can specifically bind Activin A, as well as Activin AB (by virtue of its interaction with the βA subunit). Therefore, an anti-Activin A antibody or antigen-binding fragment thereof specifically binds Activin A, or Activin A and Activin AB, but does not bind other ActRIIB ligands, such as Activin B, GDF3, GDF8, BMP2, BMP4, BMP7, BMP9, BMP10, GDF11, Nodal, etc.

In some embodiments, an anti-Activin A antibody or antigen-binding fragment thereof is employed as set forth in U.S. Pat. No. 9,718,881. Exemplary anti-Activin A antibodies that can be used in the compositions and methods of the invention include, e.g., the fully-human anti-Activin antibody H4H10446P2, also known as REGN2477, (e.g., an anti-Activin A antibody comprising the heavy and light chain variable regions having amino acid sequences SEQ ID NO: 162 and SEQ ID NO: 146, respectively, as set forth in U.S. Pat. No. 9,718,881).

Table 2 sets forth heavy and light chain variable region amino acid sequence pairs of selected anti-Activin A antibodies and their corresponding antibody identifiers that can be used in the compositions and methods of the invention. The corresponding nucleic acid sequence identifiers are set forth in Table 3.

TABLE 2
anti-Activin A Amino Acid Sequence Identifiers
SEQ ID NOs:

| Antibody | | | | | | | | |
|---|---|---|---|---|---|---|---|---|
| Designation | HCVR | HCDR1 | HCDR2 | HCDR3 | LCVR | LCDR1 | LCDR2 | LCDR3 |
| H4H10423P | 393 | 395 | 397 | 399 | 401 | 403 | 405 | 407 |
| H4H10424P | 409 | 411 | 413 | 415 | 417 | 419 | 421 | 423 |
| H4H10426P | 425 | 427 | 429 | 431 | 433 | 435 | 437 | 439 |
| H4H10429P | 441 | 443 | 445 | 447 | 449 | 451 | 453 | 455 |
| H4H10430P | 457 | 459 | 461 | 463 | 465 | 467 | 469 | 471 |
| H4H10432P2 | 473 | 475 | 477 | 479 | 481 | 483 | 485 | 487 |
| H4H10433P2 | 489 | 491 | 493 | 495 | 481 | 483 | 485 | 487 |
| H4H10436P2 | 497 | 499 | 501 | 503 | 481 | 483 | 485 | 487 |
| H4H10437P2 | 505 | 507 | 509 | 511 | 481 | 483 | 485 | 487 |
| H4H10438P2 | 513 | 515 | 517 | 519 | 481 | 483 | 485 | 487 |
| H4H10440P2 | 521 | 523 | 525 | 527 | 481 | 483 | 485 | 487 |
| H4H10442P2 | 529 | 531 | 533 | 535 | 537 | 539 | 541 | 543 |
| H4H10445P2 | 545 | 547 | 549 | 551 | 537 | 539 | 541 | 543 |
| H4H10446P2 | 553 | 555 | 557 | 559 | 537 | 539 | 541 | 543 |
| H4H10447P2 | 561 | 563 | 565 | 567 | 537 | 539 | 541 | 543 |
| H4H10448P2 | 569 | 571 | 573 | 575 | 537 | 539 | 541 | 543 |
| H4H10452P2 | 577 | 579 | 581 | 583 | 537 | 539 | 541 | 543 |
| H4H10468P2 | 585 | −587 | 589 | 591 | 537 | 539 | 541 | 543 |
| H2aM10965N | 593 | 595 | 597 | 599 | 601 | 603 | 605 | 607 |

TABLE 3
anti-Activin A Nucleic Acid Sequence Identifiers
SEQ ID NOs:

| Antibody | | | | | | | | |
|---|---|---|---|---|---|---|---|---|
| Designation | HCVR | HCDR1 | HCDR2 | HCDR3 | LCVR | LCDR1 | LCDR2 | LCDR3 |
| H4H10423P | 392 | 394 | 396 | 398 | 400 | 402 | 404 | 406 |
| H4H10424P | 408 | 410 | 412 | 414 | 416 | 418 | 420 | 422 |
| H4H10426P | 424 | 426 | 428 | 430 | 432 | 434 | 436 | 438 |
| H4H10429P | 440 | 441 | 444 | 446 | 448 | 450 | 452 | 454 |
| H4H10430P | 456 | 458 | 460 | 462 | 464 | 466 | 468 | 470 |
| H4H10432P2 | 472 | 474 | 476 | 478 | 480 | 482 | 484 | 486 |
| H4H10433P2 | 488 | 490 | 492 | 494 | 480 | 482 | 484 | 486 |
| H4H10436P2 | 496 | 498 | 500 | 502 | 480 | 482 | 484 | 486 |
| H4H10437P2 | 504 | 506 | 508 | 510 | 480 | 482 | 484 | 486 |
| H4H10438P2 | 512 | 514 | 516 | 518 | 480 | 482 | 484 | 486 |
| H4H10440P2 | 520 | 522 | 524 | 526 | 480 | 482 | 484 | 486 |
| H4H10442P2 | 528 | 530 | 532 | 534 | 536 | 538 | 540 | 542 |
| H4H10445P2 | 544 | 546 | 548 | 550 | 536 | 538 | 540 | 542 |
| H4H10446P2 | 552 | 554 | 556 | 558 | 536 | 538 | 540 | 524 |
| H4H10447P2 | 560 | 562 | 564 | 566 | 536 | 538 | 540 | 542 |
| H4H10448P2 | 568 | 570 | 572 | 574 | 536 | 538 | 540 | 542 |
| H4H10452P2 | 576 | 578 | 580 | 582 | 536 | 538 | 540 | 542 |
| H4H10468P2 | 584 | 586 | 588 | 590 | 536 | 538 | 540 | 542 |
| H2aM10965N | 592 | 594 | 596 | 598 | 600 | 602 | 604 | 606 |

In one embodiment, an anti-Activin A antibody or antigen-binding fragment thereof comprises a heavy chain variable region (HCVR) having an amino acid sequence selected from the group consisting of SEQ ID NO: 393, 409, 425, 441, 457, 473, 489, 497, 505, 513, 521, 529, 545, 553, 561, 569, 577, 585, and 593, or a substantially similar sequence thereof having at least 90%, at least 95%, at least 98% or at least 99% sequence identity.

In another embodiment, an anti-Activin A antibody or antigen-binding fragment thereof comprises a light chain variable region (LCVR) having an amino acid sequence selected from the group consisting of SEQ ID NO: 401, 417, 433, 449, 465, 481, 537, and 601, or a substantially similar sequence thereof having at least 90%, at least 95%, at least 98% or at least 99% sequence identity.

In another embodiment, an anti-Activin A antibody or antigen-binding fragment thereof comprises a HCVR and LCVR (HCVR/LCVR) amino sequence pair selected from the group consisting of SEQ ID NO: 393/401, 409/417, 425/433, 441/449, 457/465, 473/481, 489/481, 497/481, 505/481, 513/481, 521/481, 529/537, 545/537, 553/537, 561/537, 569/537, 577/537, 585/537, and 593/601.

In another embodiment, an anti-Activin A antibody or antigen-binding fragment thereof comprises a heavy chain CDR3 (HCDR3) domain having an amino acid sequence selected from the group consisting of SEQ ID NO: 399, 415, 431, 447, 463, 479, 495, 503, 511, 519, 527, 535, 551, 559, 567, 575, 583, 591, and 599, or a substantially similar sequence thereof having at least 90%, at least 95%, at least 98% or at least 99% sequence identity; and a light chain CDR3 (LCDR3) domain having an amino acid sequence selected from the group consisting of SEQ ID NO: 407, 423, 439, 455, 471, 487, 543, and 607, or a substantially similar sequence thereof having at least 90%, at least 95%, at least 98% or at least 99% sequence identity.

In another embodiment, an anti-Activin A antibody or antigen-binding fragment thereof comprises a HCDR3/LCDR3 amino acid sequence pair selected from the group consisting of SEQ ID NO: 399/407, 415/423, 431/439, 447/455, 463/471, 479/487, 495/487, 503/487, 511/487, 519/487, 527/487, 535/543, 551/543, 559/543, 567/543, 575/543, 583/543, 591/543, and 599/607.

In another embodiment, an anti-Activin A antibody or antigen-binding fragment thereof comprises a heavy chain CDR1 (HCDR1) domain having an amino acid sequence selected from the group consisting of SEQ ID NO: 395, 411, 427, 443, 459, 475, 491, 499, 507, 515, 523, 531, 547, 555, 563, 571, 579, 587, and 595, or a substantially similar sequence thereof having at least 90%, at least 95%, at least 98% or at least 99% sequence identity; a heavy chain CDR2 (HCDR2) domain having an amino acid sequence selected from the group consisting of SEQ ID NO: 397, 413, 429, 445, 461, 477, 493, 501, 509, 517, 525, 533, 549, 557, 565, 573, 581, 589, and 597, or a substantially similar sequence thereof having at least 90%, at least 95%, at least 98% or at least 99% sequence identity; a light chain CDR1 (LCDR1) domain having an amino acid sequence selected from the group consisting of SEQ ID NO: 403, 419, 435, 451, 467, 483, 539, and 603, or a substantially similar sequence thereof having at least 90%, at least 95%, at least 98% or at least 99% sequence identity; and a light chain CDR2 (LCDR2) domain having an amino acid sequence selected from the group consisting of WAS, AAS, ETS, DAS, DAS, AAS, GAS, and GAS, or a substantially similar sequence thereof having at least 90%, at least 95%, at least 98% or at least 99% sequence identity.

In another embodiment, an anti-Activin A antibody or antigen-binding fragment thereof comprises HCDR1-HCDR2-HCDR3-LCDR1-LCDR2-LCDR3 domains, respectively, having the amino acid sequences selected from the group consisting of: SEQ ID NOs: 395-397-8-403-WAS-407; 411-413-415-419-AAS-423; 36-429-431-435-ETS-439; 443-445-447-451-DAS-455; 459-461-463-467-DAS-471; 475-477-479-483-AAS-487; 491-493-495-483-AAS-487; 499-501-503-483-AAS-487; 507-509-511-483-AAS-487; 515-517-519-483-AAS-487; 523-525-527-483-AAS-487; 531-533-535-539-GAS-543; 547-549-551-539-GAS-543; 555-557-559-539-GAS-543 (H4H10446P2); 563-565-567-539-GAS-543; 571-573-575-539-GAS-543; 579-581-583-539-GAS-543; 587-589-591-539-GAS-543; and 595-597-599-603-GAS-607.

In another embodiment, an anti-Activin A antibody or antigen-binding fragment thereof comprises the heavy and light chain CDR domains contained within heavy and light chain variable region (HCVR/LCVR) sequence pairs selected from the group consisting of SEQ ID NO: 393/401, 409/417, 425/433, 441/449, 457/465, 473/481, 489/481, 497/481, 505/481, 513/481, 521/481, 529/537, 545/537, 553/537,561/537, 569/537, 577/537,585/537, and 593/601.

In one embodiment, an anti-Activin A antibody or antigen-binding fragment thereof comprises a HCVR having an amino acid sequence encoded by a nucleic acid sequence selected from the group consisting of SEQ ID NO:392, 408, 424, 440, 456, 472, 488, 496, 504, 512, 520, 528, 544, 552, 560, 568, 576, 584, and 592, or a substantially similar sequence thereof having at least 90%, at least 95%, at least 98% or at least 99% sequence identity.

In another embodiment, an anti-Activin A antibody or antigen-binding fragment thereof comprises a LCVR having an amino acid sequence encoded by a nucleic acid sequence selected from the group consisting of SEQ ID NO: 400, 416, 432, 448, 464, 480, 536, and 600, or a substantially similar sequence thereof having at least 90%, at least 95%, at least 98% or at least 99% sequence identity.

In another embodiment, an anti-Activin A antibody or antigen-binding fragment thereof comprises a HCVR and a LCVR, wherein the HCVR/LCVR pair has amino acid sequences encoded by a nucleic acid sequence pair selected from the group consisting of SEQ ID NO: 392/400, 408/416, 424/432, 440/448, 456/464, 472/480, 488/480, 496/480, 504/480, 512/480, 520/480, 528/536, 544/536, 552/536, 560/536, 568/536, 576/536, 584/536, and 592/600.

In another embodiment, an anti-Activin A antibody or antigen-binding fragment thereof comprises a HCDR3 domain having an amino acid sequence encoded by a nucleic acid sequence selected from the group consisting of SEQ ID NO:398, 414, 430, 446, 462, 478, 498, 502, 510, 518, 526, 534, 550, 558, 566, 574, 582, 590, and 598, or a substantially similar sequence thereof having at least 90%, at least 95%, at least 98% or at least 99% sequence identity, and a LCDR3 domain having an amino acid sequence encoded by a nucleic acid sequence selected from the group consisting of SEQ ID NO:406, 422, 435, 454, 470, 486, 542, and 606, or a substantially similar sequence thereof having at least 90%, at least 95%, at least 98% or at least 99% sequence identity. In one embodiment, the HCDR3/LCDR3 domain set has amino acid sequences encoded by a nucleic acid sequence pair selected from the group consisting of SEQ ID NO:398/406, 414/422, 430/438, 446/454, 462/470, 478/486, 494/486, 502/486, 510/486, 518/486, 526/486, 534/542, 550/542, 558/542, 566/542, 574/542, 582/542, 590/542, and 598/606.

In another embodiment, an anti-Activin A antibody or antigen-binding fragment thereof comprises HCDR1 and HCDR2 domains, and LCDR1 and LCDR2 domains, wherein the HCDR1 domain has an amino acid sequence encoded by a nucleic acid sequence selected from the group consisting of SEQ ID NO:394, 410, 426, 442, 458, 474, 490, 498, 506, 514, 522, 530, 546, 554, 562, 570, 578, 586, and 594, or a substantially similar sequence thereof having at least 90%, at least 95%, at least 98% or at least 99% sequence identity, the HCDR2 domain has an amino acid sequence encoded by a nucleic acid sequence selected from the group consisting of SEQ ID NO:396, 412, 428, 444, 460, 476, 492, 500, 508, 516, 524, 532, 548, 556, 564, 572, 580, 588, and 596, or a substantially similar sequence thereof having at least 90%, at least 95%, at least 98% or at least 99% sequence identity, the LCDR1 domain has an amino acid sequence encoded by a nucleic acid sequence selected from the group consisting of SEQ ID NO:402, 418, 434, 450, 466, 482, 538, and 602, or a substantially similar sequence thereof having at least 90%, at least 95%, at least 98% or at least 99% sequence identity, and the LCDR2 domain has an amino acid sequence encoded by a nucleic acid sequence selected from the group consisting of tgggcatct, gctgcatcc, gagacatcc, gatgcttcc, gatgcatcc, gctgcatcc, ggggcaagt, and ggtgcatcc, or a substantially similar sequence thereof having at least 90%, at least 95%, at least 98% or at least 99% sequence identity.

In another embodiment, an anti-Activin A antibody or antigen-binding fragment thereof comprises heavy and light chain CDR (HCDR1, HCDR2, HCDR3, LCDR1, LCDR2, LCDR3) domains having amino acid sequences encoded by a nucleic acid sequence set selected from the group consisting of SEQ ID NO:394/396/398/402/tgggcatct/406, 410/412/414/418/gctgcatcc/422, 426/428/430/434/gagacatcc/438, 442/444/446/450/gatgcttcc/454, 458/460/462/466/gatgcatcc/470, 474/476/478/482/gctgcatcc/486, 490/492/494/482/gctgcatcc/486, 498/500/502/482/gctgcatcc/486, 506/508/510/482/gctgcatcc/486, 514/516/518/482/gctgcatcc/486, 522/524/526/482/gctgcatcc/486, 530/532/534/538/ggggcaagt/542, 546/548/550/538/ggggcaagt/542, 554/556/558/538/ggggcaagt/542, 562/564/566/538/ggggcaagt/542, 570/572/574/538/ggggcaagt/542, 578/580/582/538/ggggcaagt/542, 586/588/590/538/ggggcaagt/542, and 594/596/598/602/ggtgcatcc/606.

In one embodiment, the anti-Activin A antibody or antigen-binding fragment thereof comprises the HCVR and LCVR (HCVR/LCVR) amino sequence pair of SEQ ID NO: 553/537 and the anti-GDF8 antibody or antigen-binding fragment thereof comprises a HCVR and LCVR (HCVR/LCVR) amino sequence pair of SEQ ID NO: 360/368.

In another embodiment, the anti-Activin A antibody or antigen-binding fragment thereof comprises HCDR1-HCDR2-HCDR3-LCDR1-LCDR2-LCDR3 domains, respectively, having the amino acid sequences of: SEQ ID NOs: 555-557-559-539-GAS-543 (H4H10446P2), and the anti-GDF8 antibody or antigen-binding fragment thereof comprises HCDR1-HCDR2-HCDR3-LCDR1-LCDR2-LCDR3 domains, respectively, having the amino acid sequences of: SEQ ID NOs: 362/364/366/370/TTS/374 (e.g., H4H1657N2).

In another embodiment, an anti-Activin A antibody or antigen-binding fragment thereof has a modified glycosylation pattern. In some applications, modification to remove undesirable glycosylation sites may be useful, or an antibody lacking a fucose moiety present on the oligosaccharide chain, for example, to increase antibody dependent cellular cytotoxicity (ADCC) function (Shield et al. (2002) JBC 277:26733). In other applications, modification of a galactosylation can be made in order to modify complement dependent cytotoxicity (CDC).

The fully-human anti-Activin A and/or anti-GDF8 antibodies described herein may comprise one or more amino acid substitutions, insertions and/or deletions in the framework and/or CDR regions of the heavy and light chain variable domains as compared to the corresponding germline sequences. Such mutations can be readily ascertained by comparing the amino acid sequences disclosed herein to germline sequences available from, for example, public antibody sequence databases. The compositions and methods of the invention use, in additional embodiments, antibodies and antigen-binding fragments thereof that are derived from any of the amino acid sequences disclosed herein, wherein one or more amino acids within one or more framework and/or CDR regions are back-mutated to the corresponding germline residue(s) or to a conservative amino acid substitution (natural or non-natural) of the corresponding germline residue(s) (such sequence changes are referred to herein as “germline back-mutations”). A person of ordinary skill in the art, starting with the heavy and light chain variable region sequences described herein, can easily produce numerous antibodies and antigen-binding fragments that comprise one or more individual germline back-mutations or combinations thereof. In certain embodiments, all of the framework and/or CDR residues within the VH and/or VL domains are mutated back to the germline sequence. In other embodiments, only certain residues are mutated back to the germline sequence, e.g., only the mutated residues found within the first 8 amino acids of FR1 or within the last 8 amino acids of FR4, or only the mutated residues found within CDR1, CDR2 or CDR3. Furthermore, the antibodies and antigen-binding fragments used in the compositions and methods of the invention may contain any combination of two or more germline back-mutations within the framework and/or CDR regions, i.e., wherein certain individual residues are mutated back to the germline sequence while certain other residues that differ from the germline sequence are maintained. Once obtained, antibodies and antigen-binding fragments that contain one or more germline back-mutations can be easily tested for one or more desired property such as, improved binding specificity, increased binding affinity, improved or enhanced antagonistic or agonistic biological properties (as the case may be), reduced immunogenicity, etc. Antibodies and antigen-binding fragments obtained in this general manner are encompassed within the invention.

The compositions and methods of the invention use, in additional embodiments, anti-GDF8 antibodies and/or anti-Activin A antibodies (or antigen-binding fragments thereof) comprising variants of any of the HCVR, LCVR, and/or CDR amino acid sequences described herein having one or more conservative substitutions. For example, anti-GDF8 antibodies and/or anti-Activin A antibodies used in the compositions and methods of the invention have, in some embodiments, HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences described herein.

### Bispecific Antibodies

Bispecific antibodies (bsAbs) combine specificities of two antibodies and simultaneously bind different antigens or epitopes. Two or more antigen-recognizing elements are engineered into a single antibody. In one embodiment of the methods of the invention, the composition comprises an antibody comprising a GDF8-specific binding domain and an Activin A-specific binding domain. The term (antigen)“-specific binding domain,” as used herein, includes polypeptides comprising or consisting of: (i) an antigen-binding fragment of an antibody molecule, (ii) a peptide that specifically interacts with a particular antigen (e.g., a peptibody), and/or (iii) a ligand-binding portion of a receptor that specifically binds a particular antigen. For example, included are bispecific antibodies with one arm comprising a first heavy chain variable region/light chain variable region (HCVR/LCVR) pair that specifically binds GDF8 and another arm comprising a second HCVR/LCVR pair that specifically binds Activin A.

Bispecific antibodies can be prepared according to known methods, including chemical cross-linking, hybrid hybridomas/quadromas, knobs into holes, CrossMab, dual-variable-domain immunoglobulin, recombinant engineering (tandem single chain variable fragments/diabodies), and dock and lock. Other exemplary bispecific formats that can be used in the context of the present invention include, without limitation, e.g., IgG-scFv fusions, dual variable domain (DVD)-Ig, common light chain, CrossFab, (SEED) body, leucine zipper, Duobody, IgG1/IgG2, dual acting Fab (DAF)-IgG, and Mab2 bispecific formats (see, e.g., Klein et al., mAbs 4:6, 1-11 (2012), and references cited therein, for a review of the foregoing formats). Bispecific antibodies can also be constructed using peptide/nucleic acid conjugation, e.g., wherein unnatural amino acids with orthogonal chemical reactivity are used to generate site-specific antibody-oligonucleotide conjugates, which then self-assemble into multimeric complexes with defined composition, valency and geometry. (See, e.g., Kazane et al., J Am Chem Soc. 135(1):340-346 (2013)).

### Specific Binding

The term “specifically binds” or the like, as used herein, means that an antigen-specific binding protein, or an antigen-specific binding domain, forms a complex with a particular antigen characterized by a dissociation constant (K_{D}) of 500 pM or less, and does not bind other unrelated antigens under ordinary test conditions. “Unrelated antigens” are proteins, peptides or polypeptides that have less than 95% amino acid identity to one another. Methods for determining whether two molecules specifically bind one another are well known in the art and include, for example, equilibrium dialysis, surface plasmon resonance, and the like. For example, an antigen-specific binding protein or an antigen-specific binding domain, as used in the context of the present invention, includes antibodies or antigen-binding fragments thereof that bind a particular antigen (e.g., GDF8, Activin A) or a portion thereof with a K_{D} of less than about 500 pM, less than about 400 pM, less than about 300 pM, less than about 200 pM, less than about 100 pM, less than about 90 pM, less than about 80 pM, less than about 70 pM, less than about 60 pM, less than about 50 pM, less than about 40 pM, less than about 30 pM, less than about 20 pM, less than about 10 pM, less than about 5 pM, less than about 4 pM, less than about 2 pM, less than about 1 pM, less than about 0.5 pM, less than about 0.2 pM, less than about 0.1 pM, or less than about 0.05 PM, as measured in a surface plasmon resonance assay.

Antibody binding (of antigen) can be quantitated in terms of K_{D}, a measurement of affinity. The lower the K_{D} value, the higher the binding affinity of the antibody. The term “K_{D}”, as used herein, is intended to refer to the equilibrium dissociation constant of a particular antibody-antigen interaction. Surface plasmon resonance can be used to measure ligand binding, for example, antibody-antigen interaction.

The term “surface plasmon resonance”, as used herein, refers to an optical phenomenon that allows for the analysis of real-time biospecific interactions by detection of alterations in protein concentrations within a biosensor matrix, for example using the BIACORE™ system (Pharmacia Biosensor AB, Uppsala, Sweden and Piscataway, N.J.).

The term “epitope” includes any determinant, preferably a polypeptide determinant, capable of specific binding to an immunoglobulin or T-cell receptor. In certain embodiments, epitope determinants include chemically active surface groupings of molecules such as amino acids, sugar side chains, phosphoryl groups, or sulfonyl groups, and, in certain embodiments, may have specific three-dimensional structural characteristics, and/or specific charge characteristics. An epitope is a region of an antigen that is bound by an antibody. In certain embodiments, an antibody is said to specifically bind an antigen when it preferentially recognizes its target antigen in a complex mixture of proteins and/or macromolecules. For example, an antibody is said to specifically bind an antigen when the K_{D} is less than or equal to 10⁻⁸ M, less than or equal to 10⁻⁹ M, or less than or equal to 10⁻¹⁰ M.

### Preparation of Human Antibodies

Methods for generating monoclonal antibodies, including fully human monoclonal antibodies are known in the art. Any such known methods can be used in the context of the invention to make human antibodies that specifically bind to GDF8 and/or to Activin A.

Using VELOCIMMUNE™ technology or any other known method for generating monoclonal antibodies, high affinity chimeric antibodies to GDF8 and/or Activin A are initially isolated having a human variable region and a mouse constant region. As in the experimental section below, the antibodies are characterized and selected for desirable characteristics, including affinity, selectivity, epitope, etc. The mouse constant regions are replaced with a desired human constant region to generate the fully human antibody of the invention, for example wild-type or modified IgG1 or IgG4. While the constant region selected may vary according to specific use, high affinity antigen-binding and target specificity characteristics reside in the variable region.

In general, the antibodies used in the methods of the instant invention possess very high affinities, typically possessing K_{D} of from about 10⁻¹² through about 10⁻⁹ M, when measured by binding to antigen either immobilized on solid phase or in solution phase.

### Pharmaceutical Compositions and Methods of Administration

The present invention includes methods for altering body composition of a subject. As used herein, the phrase “altering body composition” refers to a change in one or more of lean mass, fat mass, and/or bone mass in a subject. In some embodiments, body composition in a subject may be altered by administering an effective amount of a GDF8 inhibitor and an Activin A inhibitor to the subject. Lean mass may be, for example, thigh muscle volume, appendicular lean body mass, or total lean mass, etc. In some aspects, the thigh muscle volume may refer to thigh muscle tissue volume excluding intramuscular adipose tissue and large vessels. In some aspects, the thigh muscle volume may refer to thigh muscle tissue volume including intramuscular adipose tissue and large vessels. In some aspects, the appendicular lean body mass may be calculated by, for example, aLBM equation. In some aspects, the appendicular lean mass may be calculated by the sum of lean mass of arms and legs. Fat mass may be, for example, total fat mass, android fat mass, sum of intramuscular and perimuscular adipose tissue (IMAT), subcutaneous adipose tissue volume, sum of fat mass of arms and legs, thigh intramuscular adipose tissue, etc. Bone mass may be, for example, total bone mineral density (BMD) mass, total bone mineral content (BMC) mass, etc. In some embodiments, alteration of body composition comprises an increase in muscle mass and/or a reduction of fat mass. In some embodiments, alteration of body composition comprises an increase in muscle mass and a reduction of fat mass simultaneously. In some embodiments, alteration of body composition comprises an increase in muscle mass and a reduction of fat mass simultaneously, without reduction in bone mass. In some embodiments, alteration of body composition comprises an increase in bone mineral content mass. In some embodiments, alteration of body composition comprises a decrease in total fat mass, android fat mass, and/or subcutaneous fat mass. In some embodiments, alteration of body composition comprises a decrease in total fat mass, android fat mass, and/or subcutaneous fat mass, without a reduction in thigh intramuscular adipose tissue volume.

The present invention includes methods for altering body composition, for example, inducing a reduction in fat mass in a subject and methods for treating a disease or disorder characterized by increased fat mass, comprising administering a first composition comprising an effective amount of a GDF8 inhibitor and a second composition comprising an effective amount of an Activin A inhibitor to the subject. The first and second compositions can be administered concurrently or sequentially to the subject. The first and second compositions can also be combined into a third composition prior to administration. Thus, in certain embodiments, a composition comprising both a GDF8 inhibitor and an Activin A inhibitor can be administered to a subject. The GDF8 inhibitor in such a composition can, for example, be an anti-GDF8 antibody. The Activin A inhibitor in such a composition can, for example, be an anti-Activin A antibody.

The pharmaceutical compositions of the invention are formulated with suitable carriers, excipients, and other agents that provide suitable transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the invention, provided that the active ingredient in the formulation is not inactivated by the formulation and the formulation is physiologically compatible and tolerable with the route of administration. See also Powell et al. “Compendium of excipients for parenteral formulations” PDA (1998) J Pharm Sci Technol 52:238-311.

Various delivery systems are known and can be used to administer the pharmaceutical compositions of the present invention, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of administration include, but are not limited to, intradermal, intramuscular, intraperitoneal, intravenous, subcutaneous, intranasal, epidural, and oral routes. The compositions may be administered by any convenient route, for example by infusion or bolus injection, by absorption through epithelial or mucocutaneous linings (e.g., oral mucosa, rectal and intestinal mucosa, etc.) and may be administered together with other biologically active agents.

A pharmaceutical composition of the present invention can be delivered subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present invention. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ I, Il and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (Sanofi-Aventis, Frankfurt, Germany), to name only a few. Examples of disposable pen delivery devices having applications in subcutaneous delivery of a pharmaceutical composition of the present invention include, but are not limited to the SOLOSTAR™ pen (Sanofi-Aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L.P.), and the HUMIRA™ Pen (Abbott Labs, Abbott Park IL), to name only a few.

In certain situations, the pharmaceutical compositions of the present invention can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by known methods. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc.

### Dosage

The amount of active ingredient (e.g., anti-GDF8 antibody and/or anti-Activin A antibody) that can be administered to a subject is, generally, a therapeutically effective amount. The term “effective amount” is a concentration or amount of an active ingredient, for example, an antibody or antigen-binding fragment of an antibody, which results in achieving a particular stated purpose. The term “effective amount” is used interchangeably with the term “therapeutically effective amount” and signifies a concentration or amount of an active ingredient, for example, an antibody or antigen-binding fragment thereof, which is effective for achieving a stated therapeutic effect. The (therapeutically) effective amount may be determined empirically.

As used herein, the phrase “therapeutically effective amount” or “effective amount” means a dose of antigen-specific binding proteins and/or antigen-binding molecules (e.g., antibodies) that results in a detectable decrease in fat mass. The effective amount may also, in certain embodiments, result in an increase in one or more of the following parameters: body weight, muscle mass (e.g., tibialis anterior [TA] muscle mass, gastrocnemius [GA] muscle mass, quadriceps [Quad] muscle mass, appendicular lean body mass, etc.), muscle volume (e.g., thigh muscle volume), muscle strength/power, and/or muscle function, and glucose tolerance.

A “therapeutically effective amount” or “effective amount” of a GDF8 inhibitor (e.g., anti-GDF8 antibody) and/or an Activin A inhibitor (e.g., anti-Activin A antibody) includes, e.g., an amount of GDF8 inhibitor and/or Activin A inhibitor that, when administered to a subject, causes a decrease in total fat mass of at least about 2% to 8%, at least 2.5% to 6%, at least 3% to 4%, or at least about 2.0%, at least about 2.5%, at least about 3.0%, or at least about 3.5%, or more. For example, a “therapeutically effective amount” or “effective amount” of a GDF8 inhibitor (e.g., anti-GDF8 antibody) and/or an Activin A inhibitor (e.g., anti-Activin A antibody) includes, e.g., an amount of GDF8 inhibitor and/or Activin A inhibitor that, when administered to a subject, causes a decrease in total fat mass of at least about 3.5% or more.

In some embodiments, a “therapeutically effective amount” or “effective amount” of a GDF8 inhibitor (e.g., anti-GDF8 antibody) and/or an Activin A inhibitor (e.g., anti-Activin A antibody) includes, e.g., an amount of GDF8 inhibitor and/or Activin A inhibitor that, when administered to a subject, causes a decrease in android fat mass of at least about 2% to 8%, at least 2.5% to 6%, at least 3% to 4%, or at least about 2.0%, at least about 2.5%, at least about 3.0%, or at least about 3.5%, or more. For example, a “therapeutically effective amount” or “effective amount” of a GDF8 inhibitor (e.g., anti-GDF8 antibody) and/or an Activin A inhibitor (e.g., anti-Activin A antibody) includes, e.g., an amount of GDF8 inhibitor and/or Activin A inhibitor that, when administered to a subject, causes a decrease in android fat mass of at least about 3.5%.

In certain embodiments, the amount also results in an increase in TA or GA muscle mass of at least 2%, 5%, 10%, 15%, 20%, 25%, 30%, 40%, 50%, 60% or more, compared to control treated subjects.

In some embodiments, a “therapeutically effective amount” or “effective amount” of a GDF8 inhibitor (e.g., anti-GDF8 antibody) and/or an Activin A inhibitor (e.g., anti-Activin A antibody) includes, e.g., an amount of GDF8 inhibitor and/or Activin A inhibitor that, when administered to a subject, causes an increase in thigh muscle volume of at least about 2% to 8%, 2.5% to 6%, 3% to 4%, or at least 2.0%, at least 2.5%, at least 3.0%, or at least 3.5%, or more

In some embodiments, a “therapeutically effective amount” or “effective amount” of a GDF8 inhibitor (e.g., anti-GDF8 antibody) and/or an Activin A inhibitor (e.g., anti-Activin A antibody) includes, e.g., an amount of GDF8 inhibitor and/or Activin A inhibitor that, when administered to a subject, causes an increase in total lean body mass of at least about 2% to 8%, 2.5% to 6%, 3% to 4%, or at least 2.0%, at least 2.5%, at least 3.0%, or at least 3.5%, or more

In some embodiments, a “therapeutically effective amount” or “effective amount” of a GDF8 inhibitor (e.g., anti-GDF8 antibody) and/or an Activin A inhibitor (e.g., anti-Activin A antibody) includes, e.g., an amount of GDF8 inhibitor and/or Activin A inhibitor that, when administered to a subject, causes an increase in appendicular lean body mass of at least about 2% to 8%, 2.5% to 6%, 3% to 4%, or at least 2.0%, at least 2.5%, at least 3.0%, or at least 3.5%, or more.

In certain embodiments, the amount also results in an increase in TA or GA muscle mass of at least 2%, 3%, 5%, 10%, 15%, 20%, 25% or more, compared to control treated subjects.

In certain embodiments, a (therapeutically) effective amount of an anti-GDF8 antibody, anti-Activin A antibody, or bispecific antibody that specifically binds GDF8 and Activin A can be from about 0.05 mg to about 600 mg; e.g., about 0.05 mg, about 0.1 mg, about 1.0 mg, about 1.5 mg, about 2.0 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, or about 600 mg, of the respective antibody. The dose may vary depending upon the age and the size of a subject to be administered, target disease, conditions, route of administration, and the like. Depending on the severity of the condition, the frequency and the duration of the treatment can be adjusted.

The amount of antibody (e.g., anti-GDF8 antibody, anti-Activin A antibody, or bispecific antibody that specifically binds GDF8 and Activin A) contained within the individual doses may be expressed in terms of milligrams of antibody per kilogram of patient body weight (i.e., mg/kg). For example, the anti-GDF8 antibody, anti-Activin A antibody, and/or anti-GDF8/anti-Activin A bispecific antibody in the first, second, or third composition administered per the methods of the invention may be administered to a patient at a dose of about 0.0001 to about 50 mg/kg of patient body weight (e.g. 0.5 mg/kg, 1.0 mg/kg, 1.5 mg/kg, 2.0 mg/kg, 2.5 mg/kg, 3.0 mg/kg, 3.5 mg/kg, 4.0 mg/kg, 4.5 mg/kg, 5.0 mg/kg, 5.5 mg/kg, 6.0 mg/kg, 6.5 mg/kg, 7.0 mg/kg, 7.5 mg/kg, 8.0 mg/kg, 8.5 mg/kg, 9.0 mg/kg, 9.5 mg/kg, 10.0 mg/kg, 10.5 mg/kg, 11.0 mg/kg, 11.5 mg/kg, etc.).

The effective amount of a GDF8 inhibitor (e.g., an anti-GDF8 antibody) may, in certain embodiments, comprises a dosing regimen selected from the group consisting of at least 0.1 mg/kg to about 10 gm/kg, 1 mg/kg to about 1 gm/kg, and 10 mg/kg to 100 mg/kg. The effective amount of an Activin A inhibitor (e.g., an anti-Activin A antibody) may, in certain embodiments, comprise a dosing regimen selected from the group consisting of at least 0.1 mg/kg to about 10 gm/kg, 1 mg/kg to about 1 gm/kg, and 10 mg/kg to 100 mg/kg.

The effective amount of a GDF8 inhibitor (e.g., an anti-GDF8 antibody) may, in additional embodiments, comprise a dosing regimen selected from a group consisting of a single dose of about 0.01 to about 20 mg/kg body weight, about 0.1 to about 10 mg/kg body weight, and about 0.1 to about 5 mg/kg body weight. The effective amount of an Activin A inhibitor (e.g., an anti-Activin A antibody) may, in additional embodiments, comprise a dosing regimen selected from a group consisting of a single dose of about 0.01 to about 20 mg/kg body weight, about 0.1 to about 10 mg/kg body weight, and about 0.1 to about 5 mg/kg body weight. In one specific aspect, the effective amount of an anti-GDF8 antibody is from about 2 mg/kg-10 mg/kg, 4 mg/kg-8 mg/kg, or about 6 mg/kg body weight of subject, and the effective amount of the anti-Activin A antibody is from 0.5 mg/kg-15 mg/kg, 2 mg/kg-12 mg/kg, about 3 mg/kg, or about 10 mg/kg body weight of a subject.

The first, second, and third compositions administered per methods of the invention may, in certain embodiments, comprise equal amounts of GDF8 inhibitor (e.g., anti-GDF8 antibody) and/or Activin A inhibitor (e.g., anti-Activin A antibody). Alternatively, the amount of GDF8 inhibitor (e.g., anti-GDF8 antibody) in the composition may be less than or greater than the amount of Activin A inhibitor (e.g., an anti-Activin A antibody). The effective amount of a GDF8 inhibitor (e.g., anti-GDF8 antibody) may be lower, when in combination with an Activin A inhibitor (e.g., an anti-Activin A antibody), than in a separate composition. The effective amount of an Activin A inhibitor (e.g., an anti-Activin A antibody) may be lower, when in combination with a GDF8 inhibitor (e.g., anti-GDF8 antibody), than in a separate composition. A person of ordinary skill in the art, using routine experimentation, will be able to determine the appropriate amounts of the individual components in the compositions necessary to produce a desired therapeutic effect.

### Aspects of the Disclosure

The disclosure provides compositions, kits, and methods of using GDF8 inhibitors and Activin A inhibitors to reduce fat mass (induce a reduction in fat mass) in a subject. The disclosure also provides compositions, kits, and methods of using GDF8 inhibitors and Activin A inhibitors to treat diseases, disorders, and/or conditions associated with or characterized by increased fat mass in a subject. In preferred embodiments, the GDF8 inhibitor is an antibody or antigen-binding fragment thereof that specifically binds GDF8.

### Therapeutic Methods

The present invention includes methods for altering body composition, for example, methods for inducing a reduction in fat mass in a subject, methods for increasing muscle mass in a subject, and methods for treating a disease or disorder characterized by increased fat mass, by specifically binding GDF8 and/or Activin A. For example, the present invention includes methods for inducing a reduction in fat mass in a subject, inducing an increase in muscle mass in a subject, and methods for treating a disease or disorder characterized by increased fat mass in a subject, by administering to the subject i) a composition comprising an anti-GDF8 antibody and a composition comprising an anti-Activin A antibody or ii) a composition comprising both an anti-GDF8 antibody and an anti-Activin A antibody or iii) a composition comprising a bispecific antibody comprising a first variable domain comprising a HCVR/LCVR pair that specifically binds GDF8 and a second variable domain comprising a HCVR/LCVR pair that specifically binds Activin A. Any of the GDF8 inhibitors and/or Activin A inhibitors disclosed or referred to herein can be used in the context of these aspects of the invention.

In methods comprising administering a GDF8 inhibitor and an Activin A inhibitor to a subject, the GDF8 inhibitor (for example, an anti-GDF8 antibody) and the Activin A inhibitor (for example, an anti-Activin A antibody) may be administered to the subject at the same or substantially the same time, e.g., in a single therapeutic dosage (third composition) or in two separate dosages (first and second compositions), which are administered simultaneously or within less than about 5 minutes of one another. Alternatively, the GDF8 inhibitor and the Activin A inhibitor (first and second compositions) may be administered to the subject sequentially, e.g., in separate therapeutic dosages separated in time from one another by more than about 5 minutes.

The reduction of fat mass in the subject of the methods according to the invention can be a reduction in total fat mass as measured by DXA (Dual-energy X-ray absorptiometry).

In another embodiment, the reduction of fat mass in the subject of the methods according to the invention is a reduction in android fat mass (i.e., visceral fat associated with the upper/central body) as measured by DXA (Dual-energy X-ray absorptiometry). In android obesity, the subject stores fat around his or her abdominal region. Android obesity can also be manifested in other areas of the upper trunk like the upper chest (front or back) nape area of the neck, and even the shoulders. Subjects who are android obese are at greater risk for obesity-related diseases/disorders like heart disease, and metabolic syndrome. The likelihood of developing gout, arterial-related diseases (due to high blood pressure) and many kinds of cancers are also linked to the central type of fat distribution in subjects who exhibit android obesity.

Body fat assessments are varied in precision and accuracy. Common anthropometric measures include: weight, waist circumference, and skinfold measurements using skin calipers. More complex methods include: bioelectrical impedance analysis (BIA), the BOD POD, and dual-energy X-ray absorptiometry (DEXA or DXA). DXA is especially accurate and valid, because it considers bone mineral content when estimating body fat and muscle. DEXA scans can evaluate different areas of fat distribution to determine the android/gynoid fat ratio, which is distinct from body mass index. DXA can measure total fat mass, total body muscle mass, visceral fat (fat around the organs) levels, intramuscular fat (fat between the muscles), total bone mineral density, and can even provide regional breakdowns. Finally, DXA can accurately assess the distribution of body fat associated with increased insulin resistance.

### Avoidance of Side Effects

The present invention includes methods for altering body composition, for example, inducing a reduction in fat mass in a subject, and methods for treating a disease or disorder characterized by increased fat mass, comprising administering a GDF8 inhibitor and an Activin A inhibitor to the subject, without causing adverse side effects associated with the administration of molecules which bind multiple (e.g., 3 or more) ActRIIB ligands, for example, as set forth in U.S. Pat. No. 8,871,209. For example, the clinical molecule referred to as ACE-031 (Acceleron Pharma, Inc., Cambridge, MA) is a multimer consisting of the extracellular portion of ActRIIB fused to an IgG Fc domain (this molecule is also referred to herein as “ActRIIB-Fc”). ActRIIB-Fc binds GDF8 as well as other ActRIIB ligands such as, e.g., Activin A, Activin B, GDF11, BMP9, BMP10, and TGFβ, and is known to cause various adverse side effects when administered to human patients. For example, administration of ACE-031 to postmenopausal women in a Phase Ib ascending dose study was shown to cause undesired increases in hemoglobin and decreases in FSH levels. In addition, a Phase II study of ACE-031 in pediatric patients with muscular dystrophy was discontinued due to adverse effects including nose and gum bleeding. Dilated blood vessels are also observed in patients treated with ActRIIB-Fc. Effect of ACE-031 in boys with Duchenne muscular dystrophy (DMD) demonstrated trends for increased lean body mass and reduced fat mass but non-muscle-related adverse events contributed to a decision to discontinue the study. (Campbell, et al. 2017 Muscle Nerve 55:458-464). Specifically inhibiting GDF8 and Activin A (e.g., by administering an anti-GDF8 antibody and an anti-Activin A antibody), while not inhibiting other ActRIIB ligands such as Activin B, GDF11, BMP9, BMP10, and TGFβ, results in an increase in a reduction in fat mass, without causing the adverse side effects associated with non-specific Activin-binding agents such as ActRIIB-Fc.

### Administration Regimens

According to certain embodiments of the present invention, multiple doses of the compositions of the present invention (e.g., compositions comprising a GDF8 inhibitor and/or an Activin A inhibitor, for example, an anti-GDF8 antibody and/or an anti-Activin A antibody, or a bispecific antibody against GDF8 and Activin A), may be administered to a subject over a defined time course. The methods according to this aspect of the invention comprise sequentially administering to a subject multiple doses of the composition(s) of the present invention. As used herein, “sequentially administering” means that each dose of the compositions of the present invention are administered to the subject at a different point in time, e.g., on different days separated by a predetermined interval (e.g., hours, days, weeks or months). The present invention includes methods that comprise sequentially administering to the patient an initial dose of a first and/or a second composition; or a third composition; followed by one or more secondary doses of the first and/or second composition; or the third composition; and optionally followed by one or more tertiary doses of the first and/or second composition; or the third composition.

The terms “initial dose,” “secondary doses,” and “tertiary doses,” refer to the temporal sequence of administration of the compositions of the present invention. Thus, the “initial dose” is the dose that is administered at the beginning of the treatment regimen (also referred to as the “baseline dose”); the “secondary doses” are the doses that are administered after the initial dose; and the “tertiary doses” are the doses that are administered after the secondary doses. The initial, secondary, and tertiary doses may all contain the same amount of active ingredient(s), e.g., anti-GDF8 antibody and/or anti-Activin A antibody, but will generally differ from one another in terms of frequency of administration. In certain embodiments, however, the amount of active ingredient(s) contained in the initial, secondary and/or tertiary doses will vary from one another (e.g., adjusted up or down as appropriate) during the course of treatment.

In one exemplary embodiment of the present invention, each secondary and/or tertiary dose is administered 1 to 30 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more) days after the immediately preceding dose. The phrase “the immediately preceding dose,” as used herein, means, in a sequence of multiple administrations, the dose(s) of the compositions of the present invention that are administered to a subject prior to the administration of the very next dose in the sequence, with no intervening doses.

The methods according to this aspect of the invention may comprise administering to a patient any number of secondary and/or tertiary doses of the compositions of the present invention. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 29 days after the immediately preceding dose. Similarly, in embodiments involving multiple tertiary doses, each tertiary dose may be administered at the same frequency as the other tertiary doses. For example, each tertiary dose may be administered to the patient 1 to 60 days after the immediately preceding dose. Alternatively, the frequency at which the secondary and/or tertiary doses are administered to a patient can vary over the course of the treatment regimen. The frequency of administration may also be adjusted during the course of treatment by a physician, depending on the needs of the individual patient following clinical examination.

In one embodiment, a subject may be subjected to preliminary DXA, then receive a composition comprising an anti-GDF8 antibody and an anti-Activin A antibody (or a composition comprising an anti-GDF8 antibody and a composition comprising an anti-Activin A antibody), then be subjected to follow-up DXA. If the fat mass is not measurably reduced in the follow-up DXA (in comparison with the preliminary DXA), the subject may receive the composition(s) again. Subsequent dosage amount and frequency of administration can, in an additional embodiment, be varied based on the results of the follow-up DXA.

### Combination Therapies

The methods of the present invention, according to certain embodiments, comprise administering to the subject one or more additional therapeutic agents that may be advantageously combined with the composition comprising a GDF8 inhibitor and/or an Activin A inhibitor. As used herein, the expression “in combination with” means that the additional therapeutic agent(s) is/are administered before, after, or concurrently with a pharmaceutical composition comprising a GDF8 inhibitor and/or an Activin A inhibitor. The term “in combination with” also includes sequential or concomitant administration of a GDF8 inhibitor, an Activin A inhibitor, or both and a second therapeutic agent. The term “therapeutic agent” is also meant to include a specific therapy.

The additional therapeutic agent may be, e.g., another GDF8 antagonist/inhibitor, another Activin A antagonist/inhibitor, growth factor inhibitors, immunosuppressants, metabolic inhibitors, enzyme inhibitors, and cytotoxic/cytostatic agents, an IL-1 antagonist (including, e.g., an IL-1 antagonist as set forth in U.S. Pat. No. 6,927,044), an IL-6 antagonist, an IL-6R antagonist (including, e.g., an anti-IL-6R antibody as set forth in U.S. Pat. No. 7,582,298), an IL-13 antagonist, a tumor necrosis factor (TNF) antagonist, an IL-8 antagonist, an IL-9 antagonist, an IL-17 antagonist, an IL-5 antagonist, an IgE antagonist, a CD48 antagonist, an IL-31 antagonist (including, e.g., as set forth in U.S. Pat. No. 7,531,637), a thymic stromal lymphopoietin (TSLP) antagonist (including, e.g., as set forth in US 2011/027468), interferon-gamma (IFNγ) antibiotics, topical corticosteroids, tacrolimus, pimecrolimus, cyclosporine, azathioprine, methotrexate, cromolyn sodium, proteinase inhibitors, systemic corticosteroids, systemic immunotherapy, anti-histamines, chemotherapy, light therapy, or combinations thereof.

In further embodiments, the invention features a composition, the additional therapeutic agent is selected from the group consisting of (1) 3-hydroxy-3-methylglutaryl-coenzyme A (HMG-COA) reductase inhibitors, such as cerivastatin, atorvastatin, simvastatin, pitavastatin, rosuvastatin, fluvastatin, lovastatin, pravastatin, and the like; (2) inhibitors of cholesterol uptake and/or bile acid re-absorption; (3) niacin, which increases lipoprotein catabolism4) fibrates or amphipathic carboxylic acids, which reduce low-density lipoprotein (LDL) level, improve high-density lipoprotein (HDL) and TG levels, and reduce the number of non-fatal heart attacks; and (5) activators of the LXR transcription factor that plays a role in cholesterol elimination such as 22-hydroxycholesterol, or fixed combinations such as ezetimibe plus simvastatin; a statin with a bile resin (e.g., cholestyramine, colestipol, colesevelam), a fixed combination of niacin plus a statin (e.g., niacin with lovastatin); or with other lipid lowering agents such as omega-3-fatty acid ethyl esters (for example, omacor).

In still further embodiments, the second therapeutic agent is selected from one or more other inhibitors/antagonists of glucagon or an inhibitor/antagonist of the glucagon receptor, as well as inhibitors of other molecules, such as inhibitors of ANGPTL8 (for example, an anti-ANGPTL8 antibody), as well as inhibitors of other molecules, such as ANGPTL3 (for example, an anti-ANGPTL3 antibody), ANGPTL4, ANGPTL5, ANGPTL6, apolipoprotein C-III (also referred to as APOC3; see for example, inhibitors of APOC3 described in U.S. Pat. No. 8,530,439, U.S. Pat. No. 7,750,141, U.S. Pat. No. 7,598,227 and volanesorsen, also referred to as ISIS-APOCIIIRx) and proprotein convertase subtilisin/kexin type 9 (PCSK9), which are involved in lipid metabolism, in particular, cholesterol and/or triglyceride homeostasis. Inhibitors of these molecules include small molecules, antisense molecules and antibodies that specifically bind to these molecules and block their activity.

The additional therapeutic agent may, in further embodiments, be selected from the group consisting of analgesics, anti-inflammatory agents, including non-steroidal anti-inflammatory drugs (NSAIDS), such as Cox-2 inhibitors, and the like, so as to ameliorate and/or reduce the symptoms accompanying the underlying condition, if needed.

The additional therapeutic agent(s) may be administered prior to, concurrent with, or after the administration of the first and/or second; or third composition(s) described herein. For purposes of the present disclosure, such administration regimens may be considered the administration of an anti-GDF8 antibody and/or an anti-Activin A antibody “in combination with” a second therapeutically active component.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.), but some experimental errors and deviations should be accounted for. Unless indicated otherwise, parts are parts by weight, molecular weight is average molecular weight, temperature is in degrees Centigrade, and pressure is at or near atmospheric.

### Example 1. Anti-GDF8 Alone Increased Total Lean Mass Up to 3% in Clinical Studies: Sarcopenia Phase 2 Data

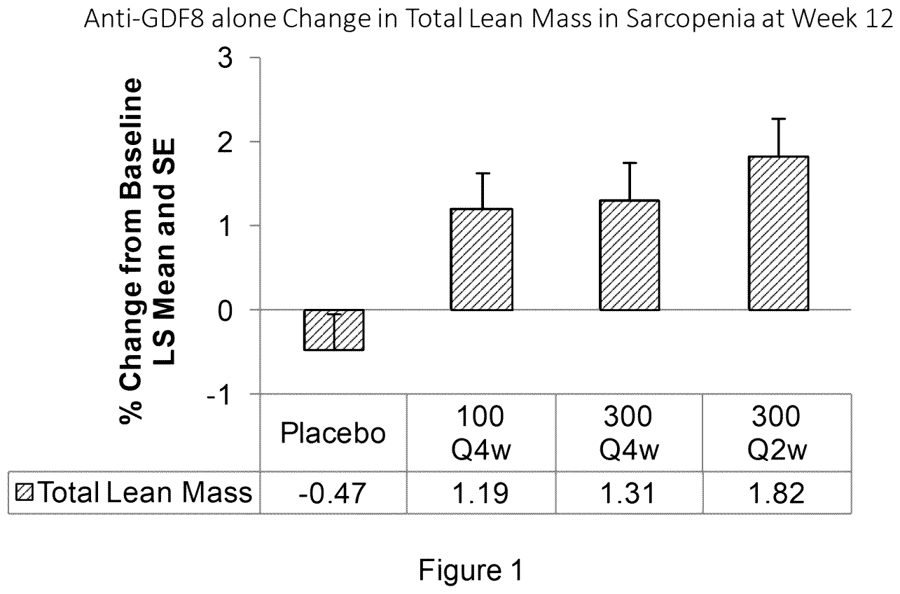

A randomized, double-blind, placebo-controlled interventional study phase 2 clinical trial was performed in patients 70 years and older including men and postmenopausal women having sarcopenia. Patients were treated 12 weeks with subcutaneous anti-GDF8 antibody REGN1033 (H4H1657N2) alone, at either 100 mg anti-GDF8 antibody, Q4W s.c. (n=62); 300 mg anti-GDF8 antibody Q4W s.c. (n=64), or 300 mg anti-GDF8 antibody Q2W s.c. (n=59), or placebo (n=65). As shown in FIG. 1 significant increase in total lean body mass compared to placebo was exhibited in patients after 12 weeks when using REGN1033 alone at each of the three doses, as shown in FIG. 1 and Table 4.

TABLE 4
Total Lean Mass % Change to Week
12 using anti-GDF8 REGN1033 alone

| | | Anti-GDF8 | | |
|---|---|---|---|---|
| | Placebo | 100 Q4W | 300 Q4W | 300 Q2W |
| N | 65 | 62 | 64 | 59 |
| Baseline | 43.6 kg | 42.9 kg | 42.7 | 42.8 |
| Mean | | | | |
| Difference vs. | — | 1.66% | 1.78% | 2.29% |
| placebo | | | | |
| P value | — | 0.0077 | 0.0043 | 0.0004 |

The effects on strength and function were varied. Anti-GDF8 was generally safe and well-tolerated (reactions, if any, were mild). Table 4 shows patients receiving REGN1033 in either 100 mg or 300 mg doses exhibited significantly increased Total lean mass as % change from placebo to week 12 data. Anti-GDF8 antibody REGN1033 alone increased total lean body mass up to 3% in the study. The 300 mg regimens also resulted in decreases in total and android fat mass.

### Example 2. Combination of Anti-GDF8 and Anti-Activin A First In-Human Single Ascending Dose Study

A randomized, double-blind placebo-controlled, ascending dose study was initiated to assess the tolerability and effects on body composition of a combination of an intravenous anti-GDF8 antibody and an anti-Activin A antibody vs. the individual components.

The primary objective of the study was to assess the safety and tolerability of an anti-Activin A antibody (e.g., H4H10446P2=REGN2477) alone and combined with an anti-GDF8 antibody (e.g., H4H1657N2=REGN1033) in healthy postmenopausal women aged 45 to 70 years of age.

Secondary objectives of the study included: an assessment of the effect of REGN2477 alone, REGN1033 alone, and REGN2477+REGN1033 in combination on thigh muscle volume as measured by Magnetic Resonance Imaging (MRI), and an assessment of the effects of REGN2477 alone, REGN1033 alone and REGN2477+REGN1033 in combination on total and regional body composition as measured by dual-energy X-ray absorptiometry (DXA)

### Study Design

This study was a randomized, double-blind, placebo-controlled, ascending dose study to assess the safety, tolerability, and pharmacodynamics of intravenous REGN2477 (anti-Activin A) alone and in combination with REGN1033 (anti-GDF8) in healthy postmenopausal women.

A total of 48 subjects were randomized to 1 of the following 4 sequential ascending REGN2477 IV dose panels where 8 subjects were randomized in a 6:2 ratio into each of the first 3 Panels (Panels A, B, and C) and 24 subjects were randomized in a 1:1:1:1 ratio (6 subjects each) into Panel D.
Panel A: 4 subjects REGN1033 (6 mg/kg IV)+REGN2477 low dose (1 mg/kg IV) or 2 subjects placebo
Panel B: 4 subjects REGN1033 (6 mg/kg IV)+REGN2477 medium dose (3 mg/kg IV) or 2 subjects placebo
Panel C: 4 subjects REGN1033 (6 mg/kg IV)+REGN2477 high dose (10 mg/kg IV) or 2 subjects placebo
Panel D: REGN1033 (6 mg/kg IV+REGN2477 high dose (10 mg/kg IV) placebo, REGN2477 (10 mg/kg IV), or REGN1033 (6 mg/kg IV)

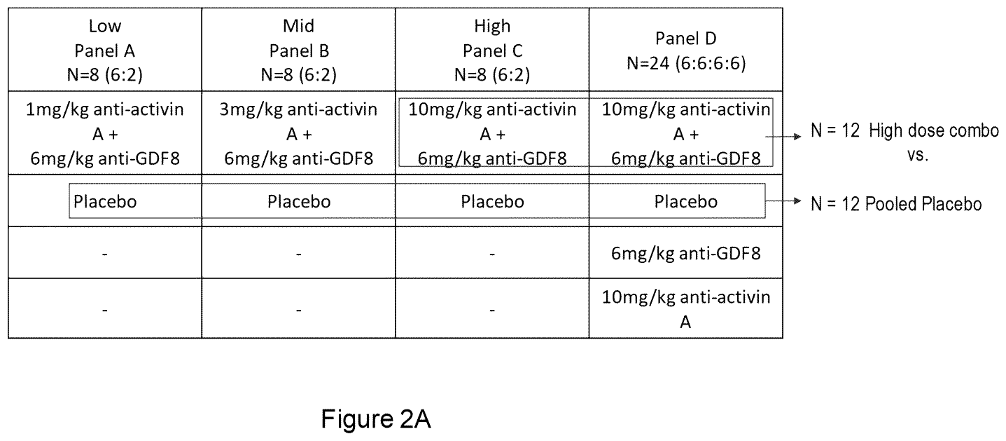

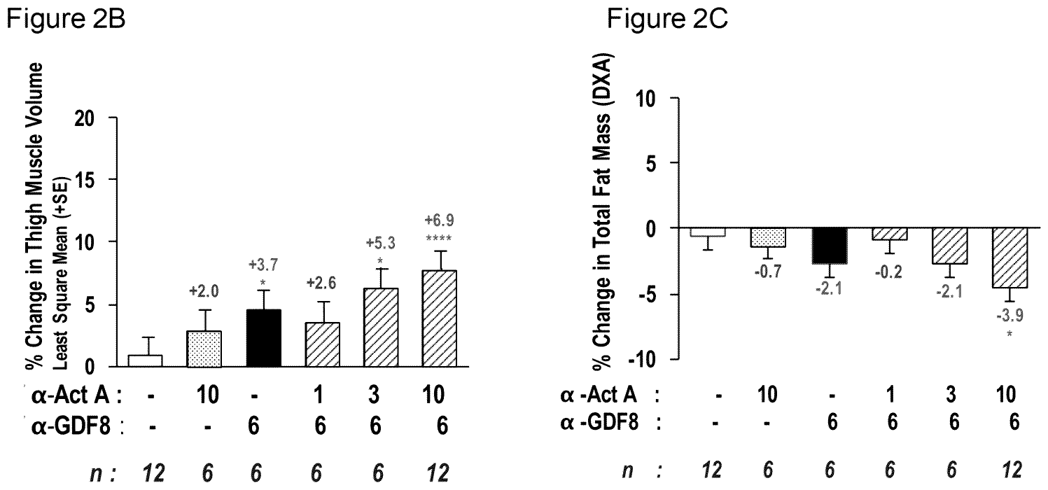

Subjects received a single intravenous dose of one or both of anti-GDF8 antibody REGN1033 and/or anti-Activin A antibody REGN2477. In the primary analyses, the placebo and high dose combination groups were pooled across panels, yielding 12 subjects on placebo and 12 on the high dose combination, as shown in FIG. 2A. The dosing schedule shown in FIG. 2A was used in studies shown in each of the subsequent FIGS. 2B to 17.

Subjects participated in a screening period of up to 28 days, followed by a baseline and treatment visit on day 1, and a follow-up period of 113 days.

### Efficacy and Safety Analysis

The full analysis set (FAS) includes all randomized subjects; it is based on the treatment allocated (as randomized). Efficacy endpoints were analyzed using the FAS. The safety analysis set (SAF) includes all randomized subjects who received any study drug; it is based on the treatment received (as treated). Treatment compliance/administration and all clinical safety variables were analyzed using the SAF.

The efficacy variables included: Thigh muscle tissue volume, excluding and including intramuscular adipose tissue and large vessels as measured by Magnetic Resonance Imaging (MRI); Total lean mass as measured by dual X-ray absorptiometry (DXA); Appendicular lean body mass (calculated by aLBM equation) as measured by DXA; and Total fat mass as measured by DXA.

The demographics and baseline characteristics of the subjects were balanced across the treatment groups, as per the below Table 5:

TABLE 5
Baseline Characteristics were balanced across treatment groups

| | | | | Anti-GDF8 + | Anti-GDF8 + | Anti-GDF8 + |
|---|---|---|---|---|---|---|
| | | | Anti-Activin A | anti-activin A | anti-activin A | anti-activin A |
| | Placebo | Anti-GDF8 | (10 mg/kg) IV | (1 mg/kg) IV | (3 mg/kg) IV | (10 mg/kg) IV |
| | (N = 12) | (N = 6) | (N = 6) | (N = 6) | (N = 6) | (N = 12) |
| Age (Years) | | | | | | |
| Median | 54 | 56 | 61 | 60 | 60 | 55.5 |
| Height (cm) | | | | | | |
| Median | 162.5 | 164.2 | 162.5 | 166.5 | 165 | 163.5 |
| Weight (kg) | | | | | | |
| Median | 68.35 | 70.5 | 69.6 | 61.35 | 67.05 | 70.4 |
| BMI (kg/m²) | | | | | | |
| Median | 26.2 | 26.55 | 26.65 | 22.9 | 25.55 | 26.6 |

### Statistical Methods

The percent change and change of efficacy variables from baseline to either week 4 or 8 in the full analysis set (FAS) were analyzed using analysis of covariance (ANCOVA) model with treatment group as fixed effect, and the baseline value as continuous covariate. Least-squares means at week 4 and week 8 for each treatment group with the corresponding standard error, confidence interval, and the p value for treatment comparisons were provided from this model. Placebo subjects were pooled across panels. Missing efficacy data was not imputed. No adjustment for multiple testing is applied in this study.

### Results

A total of 48 subjects were randomized, administered study drug and completed the study. One subject in the REGN2477+REGN1033 high dose group had an interrupted infusion of study drug due to an adverse event of ‘Infusion site swelling’.

### Efficacy

The blockade of both Activin A and GDF8 in combination increased thigh muscle volume and decreased fat mass at week 8, as shown in FIGS. 2B and 2C. The greatest effect was seen at the highest dose combination.

FIG. 2B shows a bar graph showing % change in thigh muscle volume by MRI at 8 weeks after a single dose in postmenopausal women. Numbers show changes from placebo. * indicates nominal p<0.05 vs. placebo; **** indicates nominal p<0.0001 vs. placebo. The single dose amounts as mg/kg of either anti-Activin A antibody and/or anti-GDF8 antibody are shown below the bar graph. Treatment with REGN2477+REGN1033 in medium and high dose combinations resulted in significantly increased thigh muscle volume (p<0.05; p<0.0001, respectively) in a dose-related manner compared with placebo. Subjects in high dose panel exhibited up to 7.73% as % change from baseline versus 0.88% with placebo at week 8.

FIG. 2C shows a bar graph showing % change in total fat mass by DXA at 8 weeks after a single dose in postmenopausal women. Numbers show changes from placebo. * indicates nominal p<0.05 vs. placebo; **** indicates nominal p<0.0001 vs. placebo. The single dose amounts as mg/kg of either anti-Activin A antibody and/or anti-GDF8 antibody are shown below the bar graph. Treatment with high dose REGN2477+REGN1033 combination resulted in significantly decreased total fat mass (p<0.05) compared with placebo.

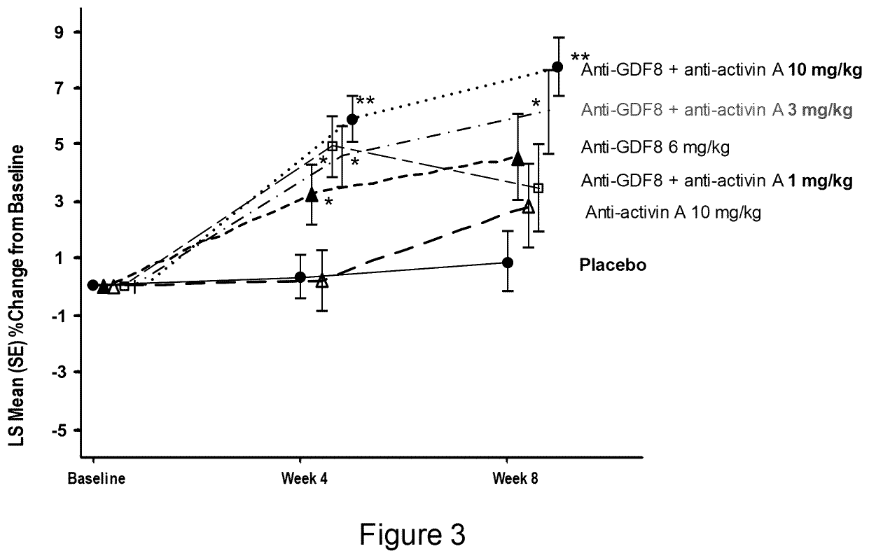

As shown in FIG. 3, anti-Activin A antibody REGN2477 combined with anti-GDF8 antibody REGN1033 led to dose-dependent increases in thigh muscle volume. The thigh muscle volume results, as measured via MRI, excluding intermuscular adipose tissue and large vessels, are likewise summarized in Table 6, below. Treatment with mid-and high dose REGN2477+REGN1033 combination resulted in significantly increased thigh muscle volume (p<0.05) compared with placebo, as shown in Table 6.

TABLE 6
Thigh Muscle Volume measured by MRI*

| | | | | | anti-GDF8 + | anti-GDF8 + | anti-GDF8 + |
|---|---|---|---|---|---|---|---|
| | | | anti- | anti-activin A | anti-activin A | anti-activin A | anti-activin A |
| | | Placebo | GDF8 | High Dose | Low Dose | Mid Dose | High Dose |
| Week 4, | N | 12 | 6 | 6 | 6 | 6 | 12 |
| % change | mean | 0.4 | 3.3 | 0.3 | 4.9 | 4.6 | 5.9 |
| Week 8, | mean | 0.9 | 4.6 | 2.9 | 3.4 | 6.2 | 7.8 |
| % change | Δ LSM vs | | 3.73 (1.819) | 1.97 (1.821) | 2.63 (1.824) | 5.31 (1.817) | 6.85 (1.484) |
| | placebo (SE) | | | | | | |
| | Nominal | | 0.0467 | 0.2846 | 0.1569 | 0.0056 | <0.0001 |
| | p-value | | | | | | |

*excluding intermuscular adipose tissue and large vessels

As shown in Table 4, treatment with REGN2477+REGN1033 in medium and high dose combinations resulted in significantly increased thigh muscle volume in a dose-related manner compared with placebo.

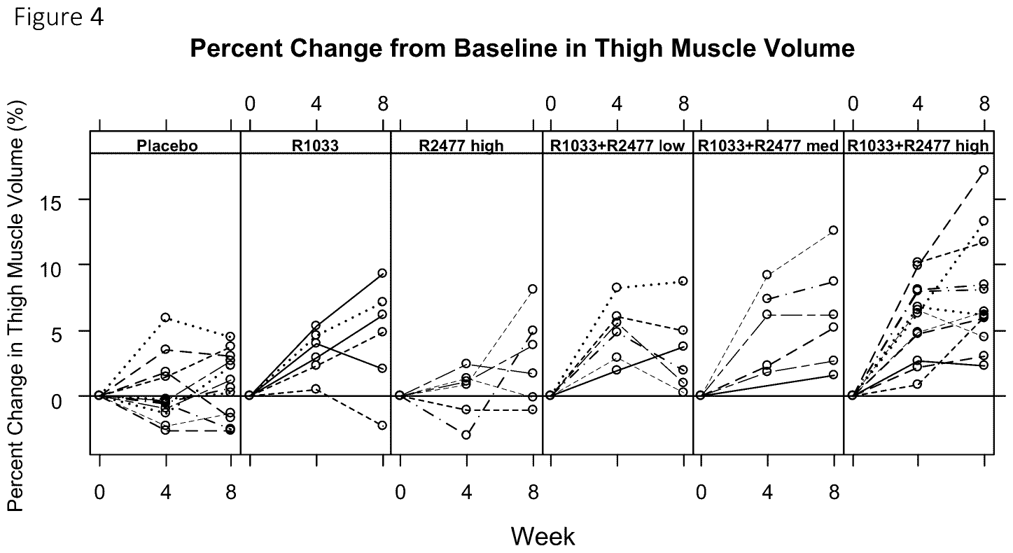

FIG. 4 shows that increases in thigh muscle volume were consistently observed in individual subjects following treatment with anti-Activin A+anti-GDF8 in combination. Within each treatment group, different lines indicate different individuals.

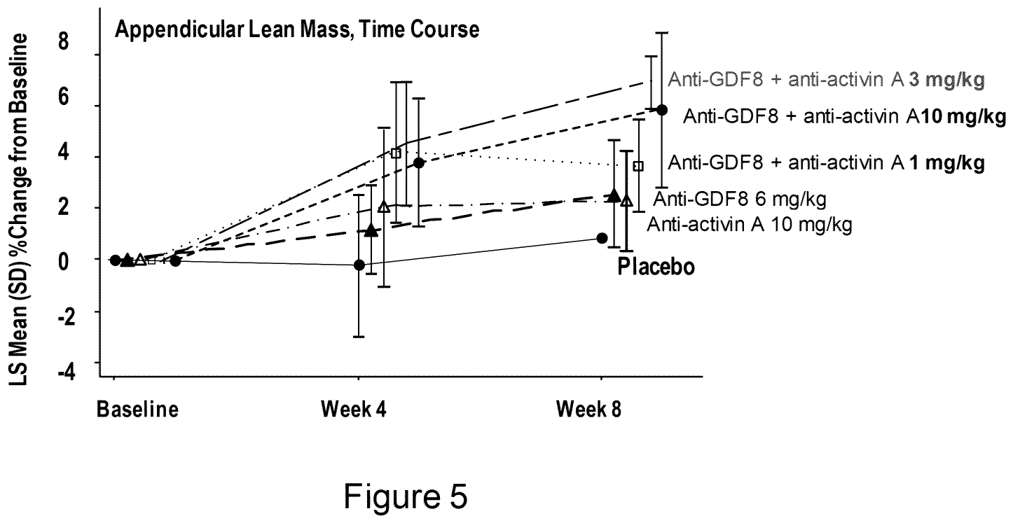

The pattern of effects on appendicular lean mass (the sum of lean mass of arms and legs) with anti-Activin A antibody REGN2477+anti-GDF8 antibody REGN1033 in combination was similar to that seen on thigh muscle volume, as shown in FIG. 5. The appendicular lean mass results, as measured via DXA, are likewise summarized in Table 7. The mid and high dosing combinations significantly increased appendicular lean mass compared to placebo, as shown in Table 7.

TABLE 7
Appendicular Lean Mass by DXA

| | | | | | Anti-GDF8 + | Anti-GDF8 + | Anti-GDF8 + |
|---|---|---|---|---|---|---|---|
| | | | | Anti-activin A | anti-activin A | anti-activin A | anti-activin A |
| | | Placebo | Anti-GDF8 | High Dose | Low Dose | Mid Dose | High Dose |
| | N | 12 | 6 | 6 | 6 | 6 | 12 |
| Baseline, kg | Mean | 17.44 | 18.10 | 18.02 | 17.92 | 18.67 | 17.32 |
| Week 4, | Mean | −0.2 | 1.2 | 2.1 | 4.2 | 4.6 | 3.8 |
| % Change | | | | | | | |
| Week 8, | Mean | 0.8 | 2.6 | 2.3 | 3.7 | 6.9 | 5.8 |
| % Change | ΔLSM vs | | 1.90 (1.266) | 1.61 (1.265) | 2.96 (1.264) | 6.39 (1.275) | 4.97 (1.031) |
| | Placebo (SE) | | | | | | |
| | Nominal | | 0.1418 | 0.2109 | 0.0242 | <0.0001 | <0.0001 |
| | p-value | | | | | | |

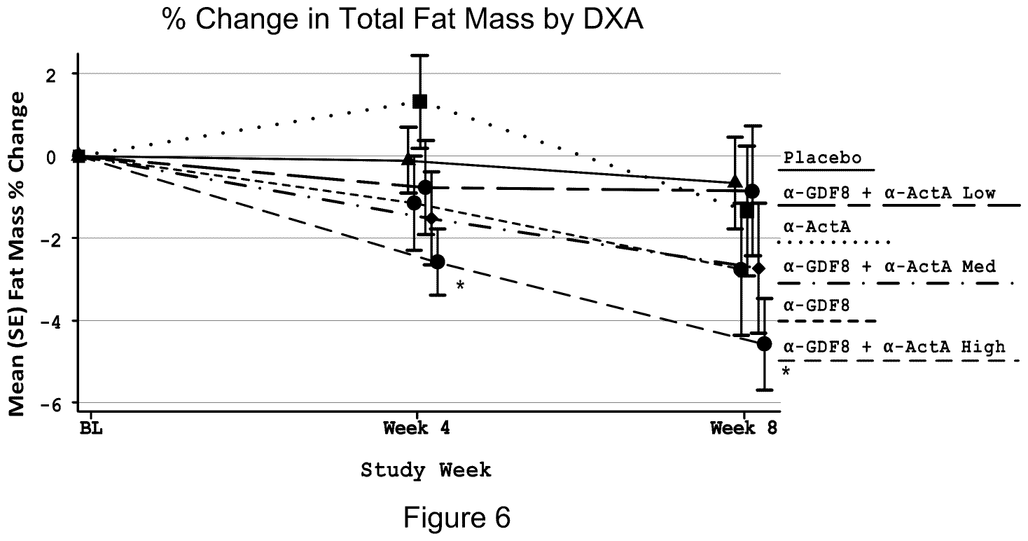

As shown in FIG. 6, blockade of both Activin A and GDF8 led to reductions in total fat mass, as assessed by DXA. The high dose combination of anti-GDF8 antibody REGN1033 and anti-Activin A antibody REGN2477 significantly reduced total fat mass by DXA compared to placebo at week 8 (*p<0.05). The total fat mass results, as measured via DXA, are likewise summarized in Table 8, below.

TABLE 8
Total Fat Mass by DXA

| | | | | anti-GDF8 + | anti-GDF8 + | anti-GDF8 + |
|---|---|---|---|---|---|---|
| | | anti-GDF8 | anti-activin A | anti-activin A | anti-activin A | anti-activin A |
| | Dose | High | High | Low | Mid | High |
| Week 8, | N | 6 | 6 | 6 | 6 | 12 |
| % Change | ΔLSM vs | −2.11 (1.96) | −0.69 (1.93) | −0.2 (1.93) | −2.08 (1.93) | −3.92 (1.58)* |
| | Placebo (SE) | | | | | |

*p < 0.05

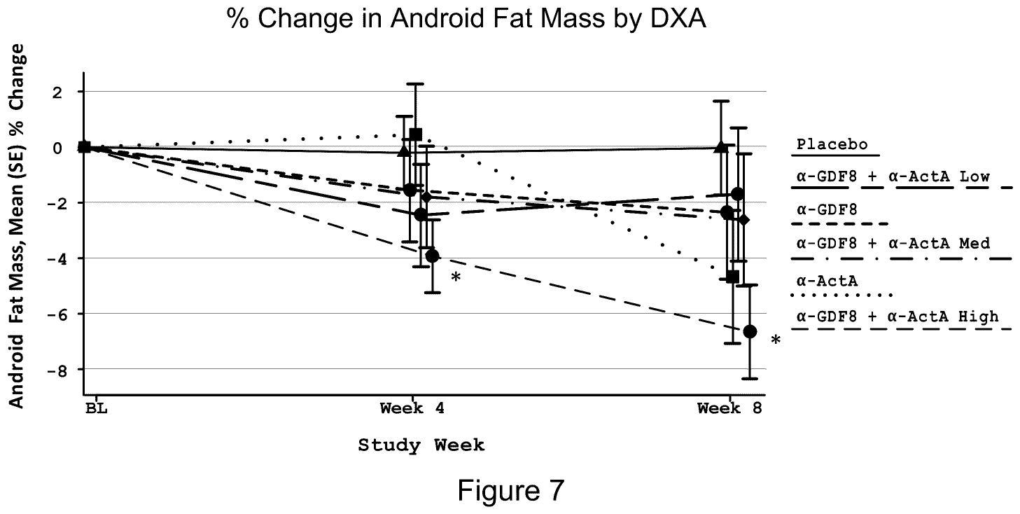

Blockade of Activin A and GDF8 was also found to be associated with decreases in android fat mass, as assessed by DXA, as shown in FIG. 7. The high dose combination of anti-GDF8 antibody REGN1033 and anti-Activin A antibody REGN2477 significantly reduced android fat mass by DXA compared to placebo at week 8 (*p<0.05).

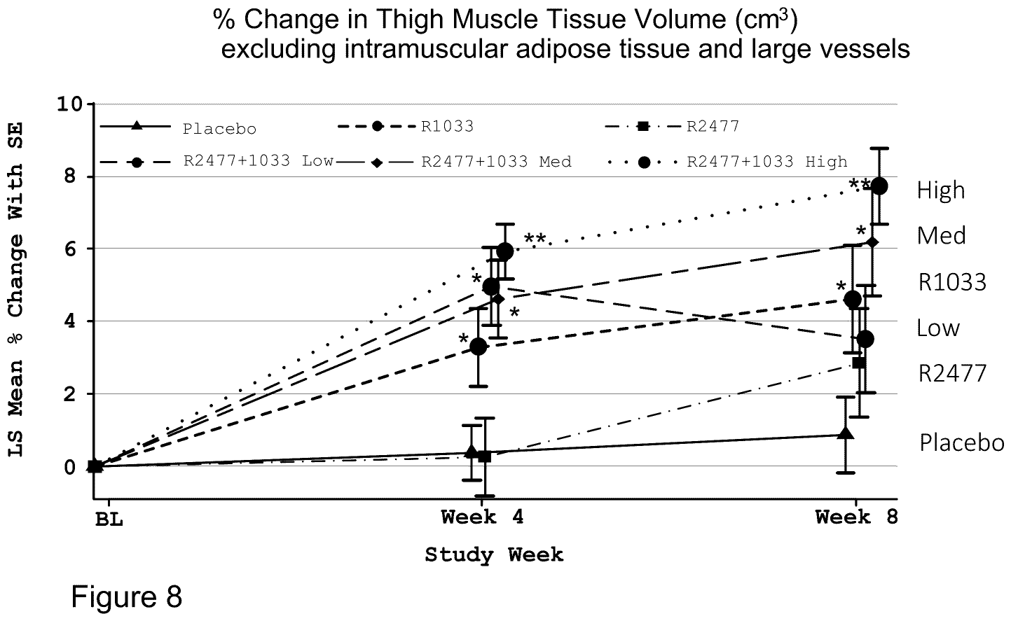

Further efficacy results are shown in FIGS. 8 to 17 and summarized in the tables, below. REGN2477+REGN1033 high, medium and low doses are shown in FIG. 2A.

Compared with placebo, REGN2477+REGN1033 medium (p<0.05) and high dose groups (p<0.001) exhibited significantly increased thigh muscle volume, excluding Intramuscular adipose tissue and large vessels (FIG. 8). Thigh muscle volume increased in the REGN2477+REGN1033 high dose group by 7.73% as compared with 0.88% in the placebo group (nominal p<0.001) at 8 weeks. Compared with placebo, REGN2477+REGN1033 at the medium dose and REGN1033 alone also significantly increased thigh muscle volume. Increases in thigh muscle volume were consistently observed in individual subjects treated with the combination in a dose responsive manner. (data not shown).

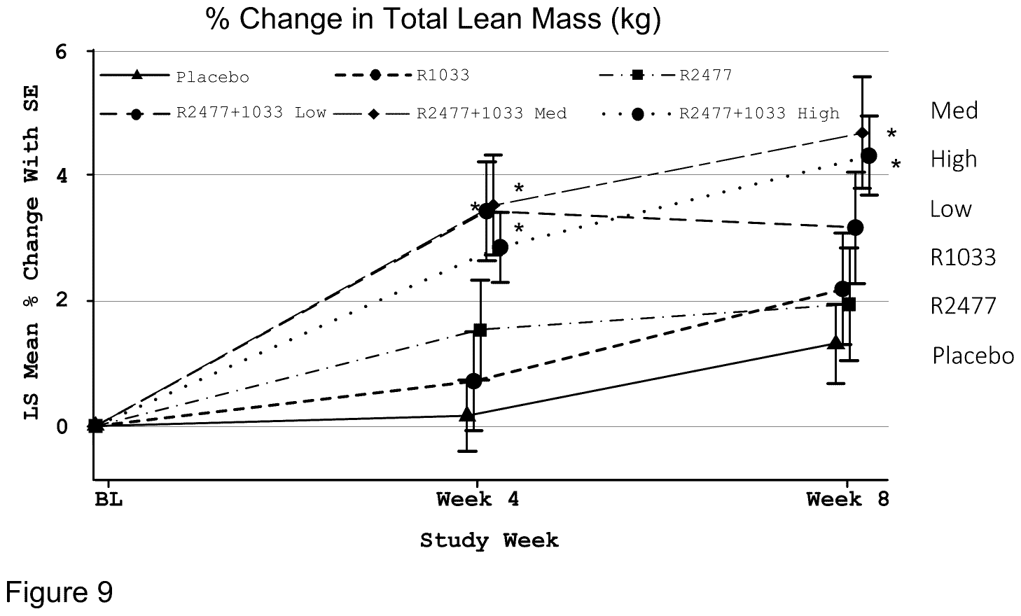

Compared with placebo, REGN2477+REGN1033 high dose group exhibited significantly increased total lean mass by DXA (p<0.05) (FIG. 9).

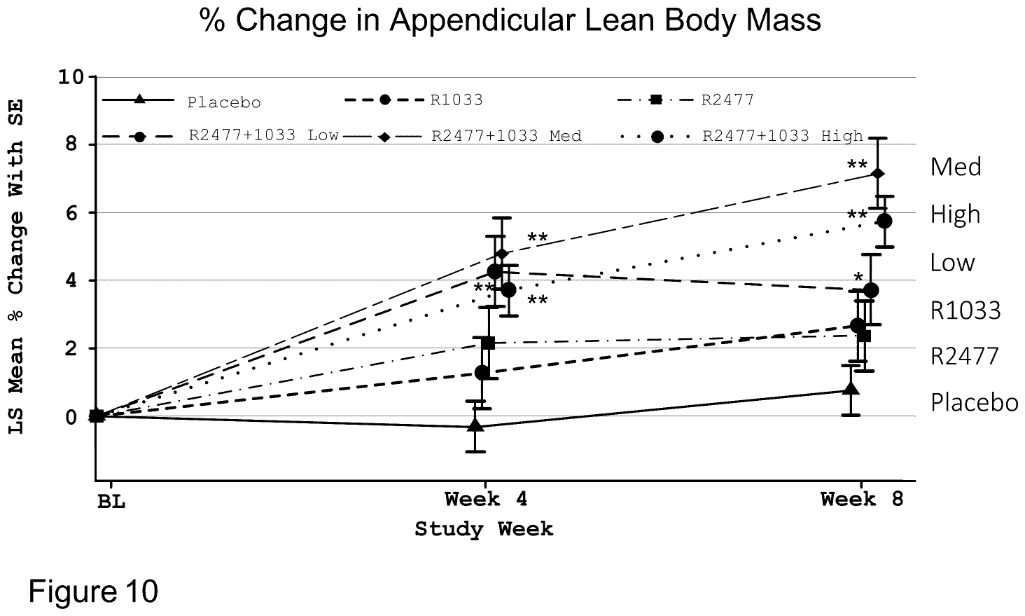

Appendicular lean body mass (calculated via aLBM equation), was significantly increased in each of the combination REGN2477+REGN1033 treatment groups compared with placebo (low dose p<0.05, medium and high dose groups p<0.001) (FIG. 10). Appendicular lean body mass increased in the REGN2477+REGN1033 medium dose group by 7.15% from baseline as compared with 0.76% in the placebo group at 8 weeks. Similarly, appendicular lean body mass increased in the REGN2477+REGN1033 high dose group by 5.7% from baseline as compared with 0.76% in the placebo group.

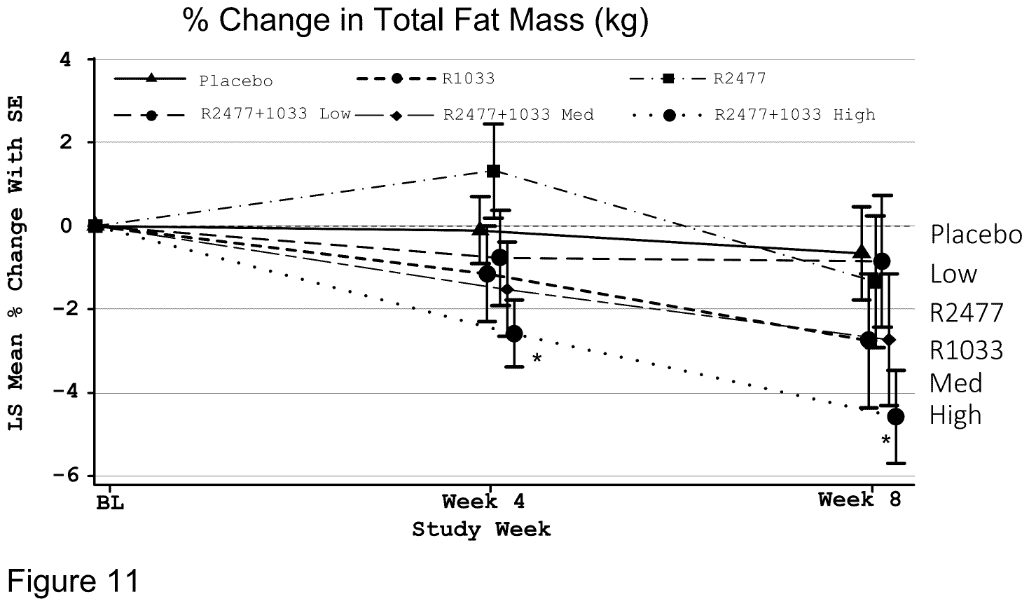

Total fat mass was significantly decreased in the high dose REGN2477+REGN1033 treatment group; total fat mass was decreased: 3.92% (high dose group) compared with placebo at 0.5% (nominal p<0.05) (FIG. 11).

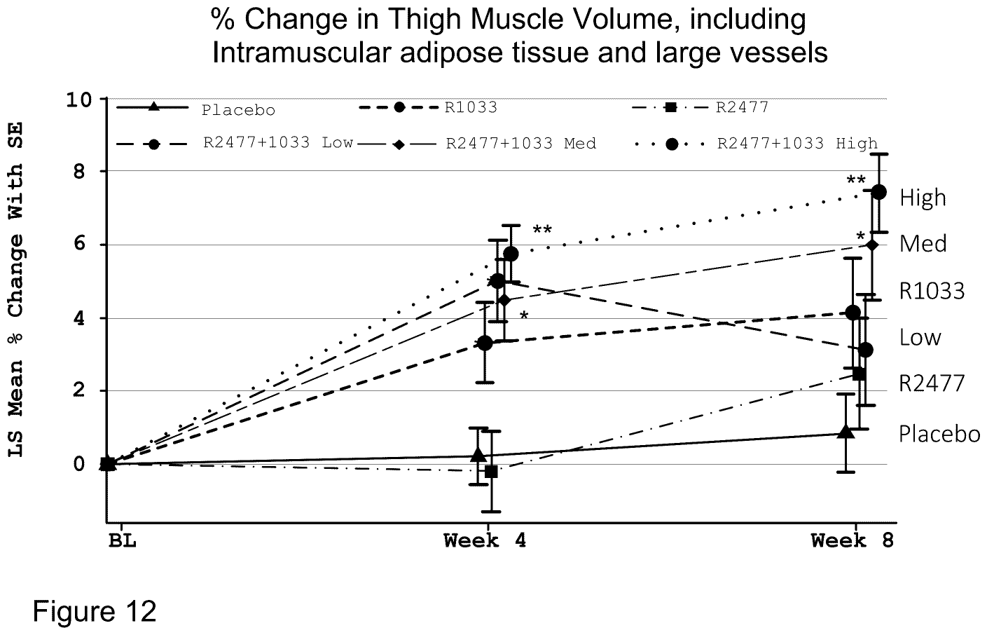

Compared to placebo, thigh muscle volume including intramuscular adipose tissue and large vessels was significantly increased in each of the medium and high dose REGN2477+REGN1033 groups and REGN1033 group at 4 weeks, and at 8 weeks (FIG. 12). Low dose REGN2477+REGN1033 also exhibited significant increase in thigh muscle volume including intramuscular adipose tissue and large vessels compared to placebo at 4 weeks.

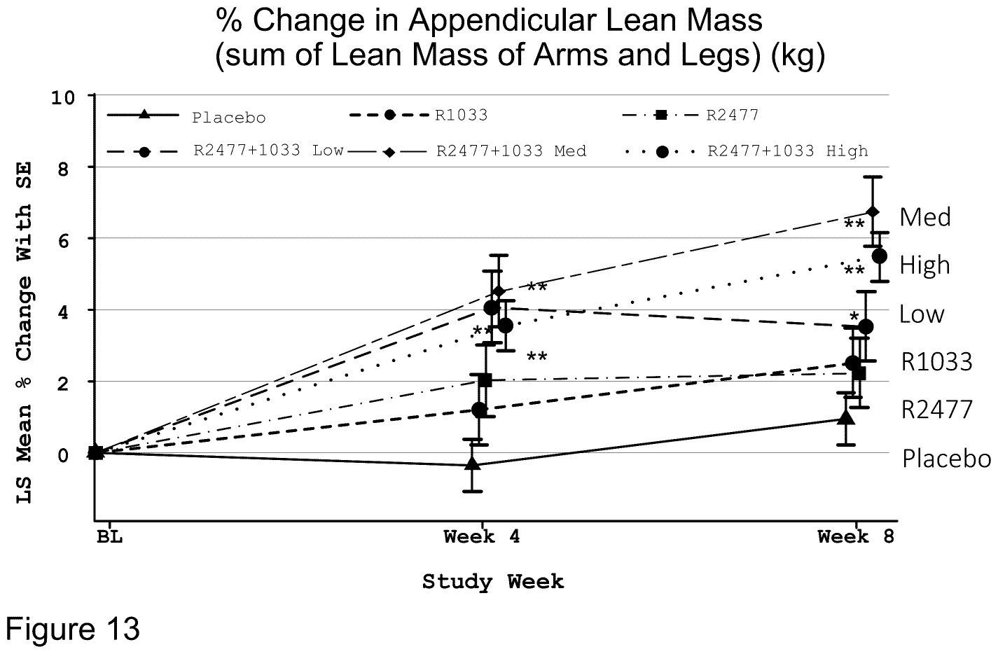

Appendicular lean mass (sum of arms and legs) was significantly increased in each of combination REGN2477+REGN1033 treatment groups (p<0.05) at 4 and 8 weeks compared to placebo (FIG. 13).

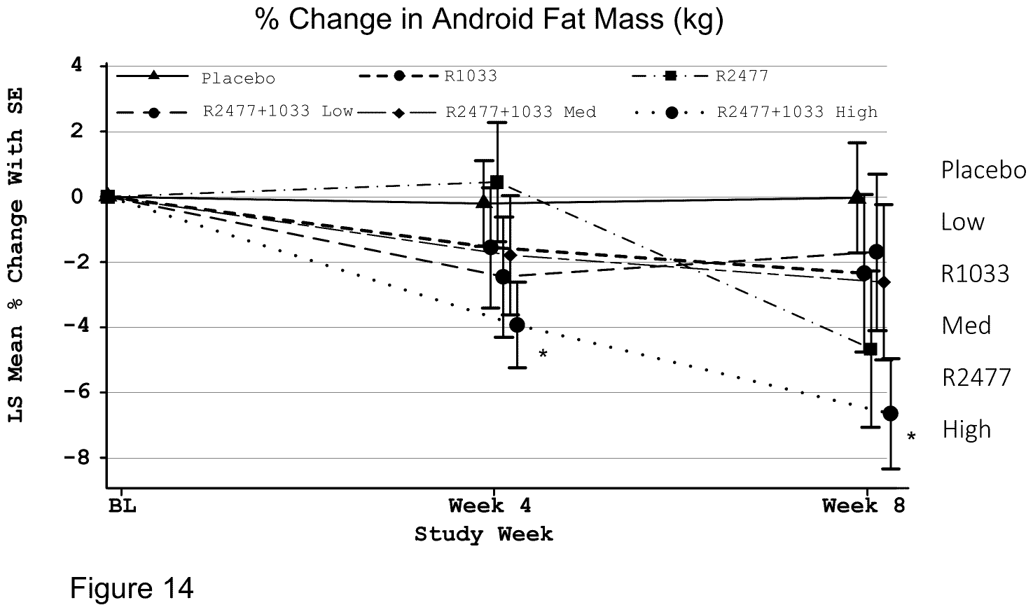

Android fat mass in the high dose REGN2477+REGN1033 treatment group was also significantly reduced (FIG. 14). Android fat mass was reduced 6.6% in the high dose REGN2477+REGN1033 group as compared to no reduction in the placebo.

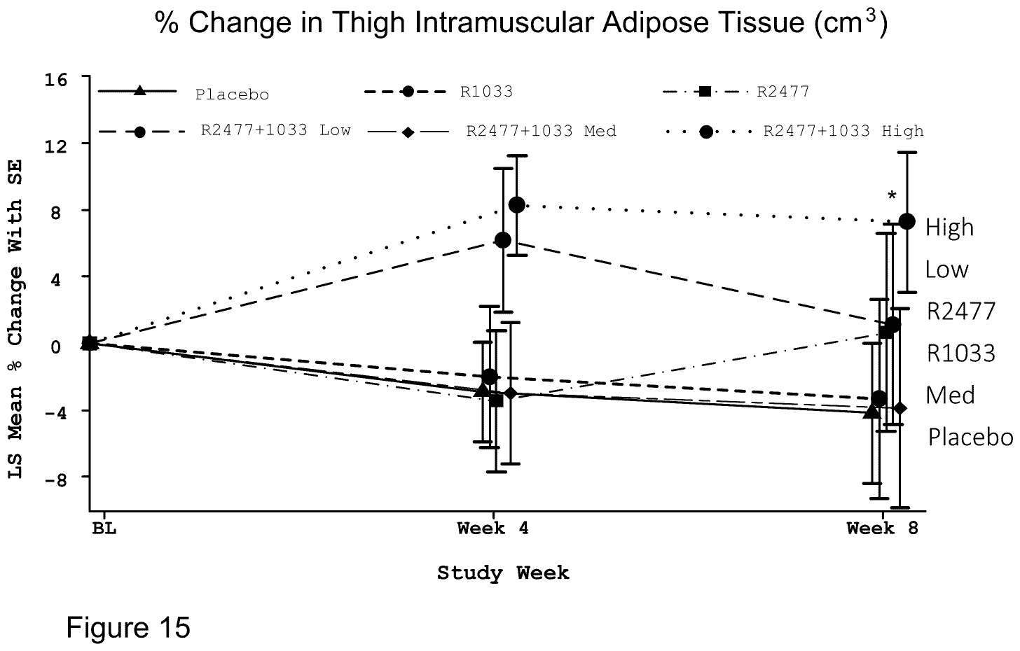

Thigh intramuscular adipose tissue volume (cm3) was significantly increased in the high dose REGN2477+REGN1033 group (p<0.05) as compared to placebo at 8 weeks (FIG. 15).

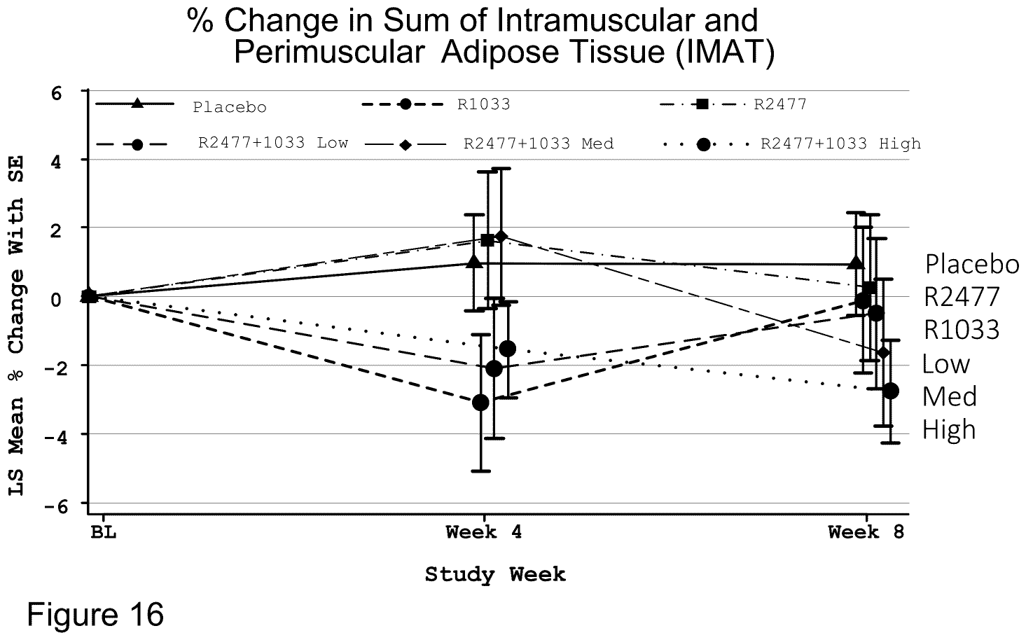

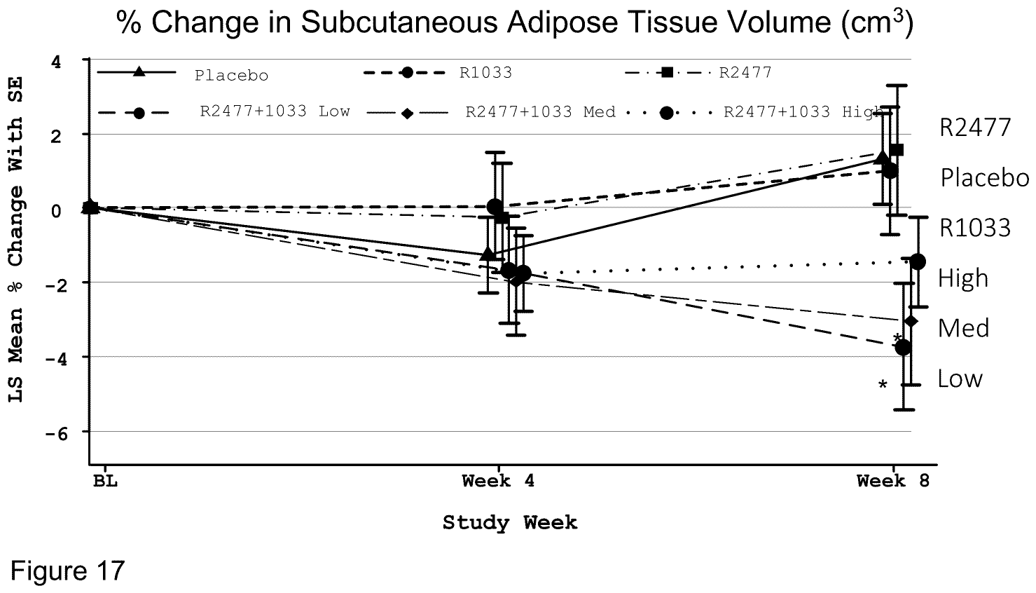

Reductions in adipose tissue in the high dose group were observed in the sum of Intramuscular and Perimuscular Adipose Tissue (IMAT) (FIG. 16) and in the low and medium dose group of subcutaneous adipose tissue. (FIG. 17). In contrast, thigh intramuscular adipose tissue was increased in the high dose group to 8% as compared with placebo with a reduction of 4%. (FIG. 17).

REGN2477+REGN1033, in the high dose group, significantly increased all of the major measures of muscle volume and lean mass compared with placebo, at both Week 4 and Week 8; the effects at Week 4 were generally less pronounced than at Week 8 (Table 9). A summary of Percent Change in Key Body Composition Measures at Week 8 (Full Analysis Set, LS Means and SE presented) is shown in Table 9, below.

TABLE 9
Summary of Change in Body Composition at Week 8

| | | | | | Low | Medium | High |
|---|---|---|---|---|---|---|---|
| | | | | | R2477 | R2477 | R2477 |
| | | | R1033 | R2477 | (1 mg/kg) | (3 mg/kg) | (10 mg/kg) |
| | | Placebo | (6 mg/kg) | (10 mg/kg) | +R1033 | +R1033 | +R1033 |
| Endpoint | | (n = 12) | (n = 6) | (n = 6) | (n = 6) | (n = 6) | (n = 12) |
| Thigh | % Change | 0.88 (1.05) | 4.61 (1.49) | 2.85 (1.49) | 3.51 (1.49) | 6.19 (1.48) | 7.73 (1.05) |
| Muscle | from | | | | | | |
| Volume | baseline | | | | | | |
| | Difference | | 3.73 (1.82)* | 1.97 (1.82) | 2.63 (1.82) | 5.31 (1.82)* | 6.85 (1.48)** |
| | from | | | | | | |
| | placebo | | | | | | |
| Total Lean | % Change | 1.31 (0.64) | 2.18 (0.89) | 1.94 (0.90) | 3.16 (0.89) | 4.67 (0.89) | 4.31 (0.63) |
| Mass by | from | | | | | | |
| DXA | baseline | | | | | | |
| | Difference | | 0.88 (1.10) | 0.63 (1.11) | 1.85 (1.10) | 3.37 (1.10)* | 3.00 (0.89)* |
| | from | | | | | | |
| | placebo | | | | | | |
| Appendicular | % Change | 0.76 (0.73) | 2.65 (1.03) | 2.37 (1.03) | 3.72 (1.03) | 7.15 (1.04) | 5.72 (0.73) |
| Lean Body | from | | | | | | |
| Mass | baseline | | | | | | |
| | Difference | | 1.90 (1.27) | 1.61 (1.26) | 2.96 (1.26)* | 6.39 (1.27)** | 4.97 (1.03)** |
| | from | | | | | | |
| | placebo | | | | | | |
| Total Fat | % Change | −0.65 (1.12) | −2.76 (1.60) | −1.34 (1.58) | −0.85 (1.58) | −2.73 (1.58) | −4.57 (1.12) |
| Mass by | from | | | | | | |
| DXA | baseline | | | | | | |
| | Difference | | −2.11 (1.96) | −0.69 (1.93) | −0.2 (1.93) | −2.08 (1.93) | −3.92 (1.58)* |
| | from | | | | | | |
| | placebo | | | | | | |

*p < 0.05;
**p < 0.001

In Table 9, above, changes from baseline and differences from placebo are Least-Squares (LS) means based on the ANCOVA model with baseline as a covariate and treatment as a fixed factor. Standard errors (SE) and p-values also taken from the ANCOVA. Nominal p-values are reported.

Bone mineral density (BMD) mass and bone mineral content (BMC) mass were measured by DXA, as shown in Table 10. At the high dose, R2477+R1033 increased Bone Mineral Content as measured by DXA, while total bone mineral density did not change (Table 10).

Sum of fat mass of arms and legs were measured as shown in Table 10. At the high dose, R2477+R1033 decreased sum of fat mass of arms and legs (p<0.05). The data from the primary endpoint analysis is shown in Table 10, below.

TABLE 10
Primary analysis of efficacy endpoints

| | | | | | R1033 + | R1033 + | R1033 + |
|---|---|---|---|---|---|---|---|
| | | | | R2477 | R2477 | R2477 | R2477 |
| | | | | (10 mg/kg) | (1 mg/kg) | (3 mg/kg) | (10 mg/kg) |
| | Analysis | Placebo | R1033 | IV | IV | IV | IV |
| Parameter | Visit | (n = 12) | (n = 6) | (n = 6) | ( n = 6) | (n = 6) | (n = 12 ) |
| Thigh Muscle | LS Mean | 0.37 (0.76) | 3.27 (1.07) | 0.27 (1.08) | 4.96 (1.08) | 4.61 (1.07) | 5.92 (0.76) |
| Tissue Volume | (SE)/Week 4 | | | | | | |
| (cm3), | LS Mean | | 2.9 (1.32)* | −0.11 (1.32) | 4.59 (1.32)* | 4.24 (1.31)* | 5.55 (1.07)** |
| Excluding Intra- | Diff (SE)/ | | | | | | |
| muscular Adipose | week 4 | | | | | | |
| Tissue and Large | LS Mean | 0.88 (1.05) | 4.61 (1.49) | 2.85 (1.49) | 3.51 (1.49) | 6.19 (1.48) | 7.73 (1.05) |
| Vessels/% change | (SE)/week 8 | | | | | | |
| | LS Mean | | 3.73 (1.82)* | 1.97 (1.82) | 2.63 (1.82) | 5.31 (1.82)* | 6.85 (1.48)** |
| | Diff (SE)/ | | | | | | |
| | week 8 | | | | | | |
| Change | LS Mean | 1.64 (3.26) | 13.95 (4.61) | −0.14 (4.63) | 20.29 (4.63) | 18.77 (4.61) | 24.5 (3.26) |
| | (SE)/week 4 | | | | | | |
| | LS Mean | | 12.32 (5.65)* | −1.78 (5.66) | 18.65 (5.67)* | 17.14 (5.64)* | 22.86 (4.61)** |
| | Diff (SE)/ | | | | | | |
| | week 4 | | | | | | |
| | LS Mean | 3.5 (4.13) | 20.42 (5.85) | 11.22 (5.87) | 14.55 (5.88) | 25.44 (5.85) | 31.95 (4.14) |
| | (SE)/week 8 | | | | | | |
| | LS Mean | | 16.91 (7.17)* | 7.71 (7.18) | 11.05 (7.19) | 21.94 (7.16)* | 28.45 (5.85)** |
| | Diff (SE)/ | | | | | | |
| | week 8 | | | | | | |
| Thigh Muscle | LS Mean | 0.21 (0.78) | 3.32 (1.1) | −0.2 (1.11) | 5 (1.11) | 4.49 (1.1) | 5.76 (0.78) |
| Volume (cm3), | (SE)/week 4 | | | | | | |
| Including | LS Mean | | 3.1 (1.35)* | −0.41 (1.36) | 4.79 (1.35)* | 4.28 (1.35)* | 5.54 (1.1)** |
| Intra-muscular | Diff (SE)/ | | | | | | |
| Adipose Tissue | week 4 | | | | | | |
| and Large | LS Mean | 0.85 (1.06) | 4.14 (1.51) | 2.47 (1.52) | 3.14 (1.51) | 5.99 (1.5) | 7.41 (1.06) |
| Vessels/ | (SE)/week 8 | | | | | | |
| % change | LS Mean | | 3.29 (1.84) | 1.62 (1.85) | 2.29 (1.85) | 5.14 (1.84)* | 6.57 (1.5)** |
| | Diff (SE)/ | | | | | | |
| | week 8 | | | | | | |
| change | LS Mean | 0.99 (3.45) | 14.76 (4.89) | −2.61 (4.94) | 21.22 (4.91) | 19.35 (4.89) | 25.22 (3.45) |
| | (SE)/week 4 | | | | | | |
| | LS Mean | | 13.77 (5.99)* | −3.6 (6.02) | 20.24 (6.01)* | 18.36 (5.98)* | 24.23 (4.88)** |
| | Diff (SE)/ | | | | | | |
| | week 4 | | | | | | |
| | LS Mean | 3.55 (4.42) | 19.54 (6.25) | 9.26 (6.32) | 13.64 (6.29) | 26.04 (6.25) | 32.27 (4.42) |
| | (SE)/week 8 | | | | | | |
| | LS Mean | | 15.99 (7.66)* | 5.72 (7.7) | 10.09 (7.69) | 22.49 (7.65)* | 28.72 (6.25)** |
| | Diff (SE)/ | | | | | | |
| | week 8 | | | | | | |
| Appendicular | LS Mean | −0.31 (0.74) | 1.26 (1.04) | 2.15 (1.04) | 4.26 (1.04) | 4.77 (1.05) | 3.7 (0.74) |
| Lean Body Mass | (SE)/ | | | | | | |
| (Calculated | week 4 | | | | | | |
| by aLBM | LS Mean | | 1.57 (1.28) | 2.46 (1.28) | 4.57 (1.28)** | 5.08 (1.29)** | 4.01 (1.04)** |
| Equation) | Diff (SE)/ | | | | | | |
| (kg)/% change | week 4 | | | | | | |
| | LS Mean | 0.76 (0.73) | 2.65 (1.03) | 2.37 (1.03) | 3.72 (1.03) | 7.15 (1.04) | 5.72 (0.73) |
| | (SE)/week 8 | | | | | | |
| | LS Mean | | 1.9 (1.27) | 1.61 (1.26) | 2.96 (1.26)* | 6.39 (1.27)** | 4.97 (1.03)** |
| | Diff (SE)/ | | | | | | |
| | week 8 | | | | | | |
| Change | LS Mean | −0.05 (0.13) | 0.23 (0.18) | 0.35 (0.18) | 0.73 (0.18) | 0.84 (0.18) | 0.63 (0.13) |
| | (SE)/week 4 | | | | | | |
| | LS Mean | | 0.28 (0.22) | 0.4 (0.22) | 0.78 (0.22)* | 0.89 (0.22)** | 0.68 (0.18)** |
| | Diff (SE)/ | | | | | | |
| | week 4 | | | | | | |
| | LS Mean | 0.11 (0.13) | 0.45 (0.19) | 0.41 (0.19) | 0.64 (0.18) | 1.28 (0.19) | 1.02 (0.13) |
| | (SE) | | | | | | |
| | LS Mean | | 0.33 (0.23) | 0.29 (0.23) | 0.53 (0.23)* | 1.16 (0.23)** | 0.91 (0.18)** |
| | Diff (SE)/ | | | | | | |
| | week 8 | | | | | | |
| Total Lean Mass | LS Mean | 0.16 (0.56) | 0.71 (0.79) | 1.53 (0.79) | 3.42 (0.79) | 3.52 (0.79) | 2.84 (0.56) |
| (kg)/% change | (SE)/week 4 | | | | | | |
| | LS Mean | | 0.55 (0.97) | 1.37 (0.98) | 3.26 (0.98)* | 3.36 (0.98)* | 2.69 (0.79)* |
| | Diff (SE)/ | | | | | | |
| | week 4 | | | | | | |
| | LS Mean | 1.31 (0.64) | 2.18 (0.89) | 1.94 (0.9) | 3.16 (0.89) | 4.67 (0.89) | 4.31 (0.63) |
| | (SE)/week 8 | | | | | | |
| | LS Mean | | 0.88 (1.1) | 0.63 (1.11) | 1.85 (1.1) | 3.37 (1.1)* | 3 (0.89)* |
| | Diff (SE)/ | | | | | | |
| | week 8 | | | | | | |
| change | LS Mean | 0.08 (0.23) | 0.25 (0.33) | 0.61 (0.33) | 1.42 (0.33) | 1.44 (0.33) | 1.14 (0.23) |
| | (SE)/week 4 | | | | | | |
| | LS Mean | | 0.17 (0.41) | 0.54 (0.41) | 1.34 (0.41)* | 1.36 (0.41)* | 1.07 (0.33)* |
| | Diff (SE)/ | | | | | | |
| | week 4 | | | | | | |
| | LS Mean | 0.53 (0.27) | 0.86 (0.37) | 0.78 (0.37) | 1.27 (0.37) | 1.92 (0.37) | 1.8 (0.26) |
| | (SE)/week 8 | | | | | | |
| | LS Mean | | 0.33 (0.46) | 0.25 (0.46) | 0.74 (0.46) | 1.39 (0.46)* | 1.26 (0.37)* |
| | Diff (SE)/ | | | | | | |
| | week 8 | | | | | | |
| Appendicular | LS Mean | −0.35 (0.74) | 1.2 (1) | 2.02 (1) | 4.06 (1) | 4.51 (1) | 3.54 (0.71) |
| Lean Mass | (SE)/week 4 | | | | | | |
| (Sum of Lean | LS Mean | | 1.56 (1.25) | 2.37 (1.24) | 4.42 (1.24)** | 4.87 (1.26)** | 3.89 (1.02)** |
| Mass of Arms | Diff (SE)/ | | | | | | |
| and Legs) | week 4 | | | | | | |
| (kg)/% change | LS Mean | 0.95 (0.72) | 2.51 (0.97) | 2.22 (0.97) | 3.53 (0.97) | 6.74 (0.98) | 5.48 (0.69) |
| | (SE)/week 8 | | | | | | |
| | LS Mean | | 1.56 (1.22) | 1.28 (1.21) | 2.58 (1.21)* | 5.79 (1.23)** | 4.53 (0.99)** |
| | Diff (SE)/ | | | | | | |
| | week 8 | | | | | | |
| change | LS Mean | −0.06 (0.14) | 0.23 (0.18) | 0.35 (0.18) | 0.73 (0.18) | 0.83 (0.19) | 0.64 (0.13) |
| | (SE)/week 4 | | | | | | |
| | LS Mean | | 0.28 (0.23) | 0.41 (0.23) | 0.79 (0.23)* | 0.89 (0.23)** | 0.69 (0.19)** |
| | Diff (SE)/ | | | | | | |
| | week 4 | | | | | | |
| | LS Mean | 0.17 (0.14) | 0.44 (0.18) | 0.4 (0.18) | 0.64 (0.18) | 1.26 (0.18) | 1.02 (0.13) |
| | (SE)/week 8 | | | | | | |
| | LS Mean | | 0.27 (0.23) | 0.23 (0.23) | 0.48 (0.23)* | 1.09 (0.23)** | 0.86 (0.19)** |
| | Diff (SE)/ | | | | | | |
| | week 8 | | | | | | |
| Total Fat Mass | LS Mean | −0.11 (0.81) | −1.15 (1.15) | 1.32 (1.14) | −0.76 (1.14) | −1.52 (1.14) | −2.58 (0.8) |
| (kg)/% change | (SE)/week 4 | | | | | | |
| | LS Mean | | −1.04 (1.41) | 1.42 (1.39) | −0.65 (1.39) | −1.41 (1.39) | −2.47 (1.14)* |
| | Diff (SE)/ | | | | | | |
| | week 4 | | | | | | |
| | LS Mean | −0.65 (1.12) | −2.76 (1.6) | −1.34 (1.58) | −0.85 (1.58) | −2.73 (1.58) | −4.57 (1.12) |
| | (SE)/week 8 | | | | | | |
| | LS Mean | | −2.11 (1.96) | −0.69 (1.93) | −0.2 (1.93) | −2.08 (1.93) | −3.92 (1.58)* |
| | Diff (SE)/ | | | | | | |
| | week 8 | | | | | | |
| change | LS Mean | 0.07 (0.21) | −0.18 (0.3) | 0.29 (0.29) | −0.29 (0.29) | −0.38 (0.29) | −0.6 (0.21) |
| | (SE)/week 4 | | | | | | |
| | LS Mean | | −0.25 (0.36) | 0.23 (0.36) | −0.36 (0.36) | −0.45 (0.36) | −0.67 (0.29)* |
| | Diff (SE)/ | | | | | | |
| | week 4 | | | | | | |
| | LS Mean | −0.04 (0.28) | −0.54 (0.4) | −0.46 (0.4) | −0.34 (0.4) | −0.75 (0.4) | −1.16 (0.28) |
| | (SE)/week 8 | | | | | | |
| | LS Mean | | −0.5 (0.49) | −0.42 (0.49) | −0.3 (0.49) | −0.71 (0.49) | −1.12 (0.4)* |
| | Diff (SE)/ | | | | | | |
| | week 8 | | | | | | |
| Android Fat Mass | LS Mean | −0.21 (1.3) | −1.57 (1.85) | 0.44 (1.84) | −2.47 (1.84) | −1.8 (1.83) | −3.94 (1.3) |
| (kg)/% change | (SE)/week 4 | | | | | | |
| | LS Mean | | −1.35 (2.26) | 0.65 (2.25) | −2.25 (2.25) | −1.59 (2.25) | −3.73 (1.83)* |
| | Diff (SE)/ | | | | | | |
| | week 4 | | | | | | |
| | LS Mean | −0.05 (1.69) | −2.35 (2.4) | −4.67 (2.39) | −1.71 (2.39) | −2.62 (2.38) | −6.65 (1.69) |
| | (SE) | | | | | | |
| | LS Mean | | −2.31 (2.93) | −4.62 (2.92) | −1.66 (2.93) | −2.57 (2.92) | −6.61 (2.38)* |
| | Diff (SE)/ | | | | | | |
| | week 8 | | | | | | |
| change | LS Mean | 0.03 (0.03) | 0.01 (0.04) | 0 (0.04) | −0.05 (0.04) | −0.04 (0.04) | −0.07 (0.03) |
| | (SE)/week 4 | | | | | | |
| | LS Mean | | −0.01 (0.05) | −0.02 (0.05) | −0.07 (0.05) | −0.06 (0.05) | −0.1 (0.04)* |
| | Diff (SE)/ | | | | | | |
| | week 4 | | | | | | |
| | LS Mean | 0.03 (0.04) | −0.01 (0.05) | −0.09 (0.05) | −0.03 (0.05) | −0.07 (0.05) | −0.13 (0.04) |
| | (SE)/week 8 | | | | | | |
| | LS Mean | | −0.04 (0.06) | −0.11 (0.06) | −0.06 (0.06) | −0.1 (0.06) | −0.16 (0.05)* |
| | Diff (SE)/ | | | | | | |
| | week 8 | | | | | | |
| Thigh Intra- | LS Mean | −2.9 (3) | −2 (4.24) | −3.44 (4.23) | 6.19 (4.28) | −2.99 (4.24) | 8.25 (2.98) |
| muscular | (SE)/week 4 | | | | | | |
| Adipose Tissue | LS Mean | | 0.9 (5.22) | −0.54 (5.17) | 9.09 (5.27) | −0.09 (5.17) | 11.15 (4.23)* |
| Volume (cm3)/ | Diff (SE)/ | | | | | | |
| % volume | week 4 | | | | | | |
| | LS Mean | −4.19 (4.21) | −3.35 (5.96) | 0.65 (5.95) | 1.14 (6.02) | −3.88 (5.96) | 7.26 (4.19) |
| | (SE)/week 8 | | | | | | |
| | LS Mean | | 0.84 (7.34) | 4.84 (7.26) | 5.33 (7.41) | 0.31 (7.27) | 11.45 (5.95) |
| | Diff (SE)/ | | | | | | |
| | week 8 | | | | | | |
| change | LS Mean | −0.09 (0.13) | −0.07 (0.18) | −0.2 (0.18) | 0.14 (0.18) | −0.12 (0.18) | 0.24 (0.13) |
| | (SE)/week 4 | | | | | | |
| | LS Mean | | 0.02 (0.22) | −0.11 (0.22) | 0.23 (0.22) | −0.03 (0.22) | 0.33 (0.18) |
| | Diff (SE)/ | | | | | | |
| | week 4 | | | | | | |
| | LS Mean | −0.17 (0.17) | −0.13 (0.24) | −0.02 (0.24) | −0.08 (0.25) | −0.22 (0.24) | 0.2 (0.17) |
| | (SE)/week 8 | | | | | | |
| | LS Mean | | 0.04 (0.3) | 0.15 (0.3) | 0.09 (0.3) | −0.05 (0.3) | 0.37 (0.24) |
| | Diff (SE)/ | | | | | | |
| | week 8 | | | | | | |
| Sum of Intra- | LS Mean | 0.98 (1.4) | −3.09 (1.98) | 1.63 (1.99) | −2.09 (2.03) | 1.75 (1.99) | −1.54 (1.4) |
| muscular and | (SE)/week 4 | | | | | | |
| Peri-muscular | LS Mean | | −4.07 (2.43) | 0.65 (2.43) | −3.07 (2.47) | 0.77 (2.43) | −2.52 (1.98) |
| Adipose Tissue | Diff (SE)/ | | | | | | |
| (IMAT)/% change | week 4 | | | | | | |
| | LS Mean | 0.94 (1.49) | −0.12 (2.12) | 0.26 (2.13) | −0.5 (2.17) | −1.63 (2.12) | −2.76 (1.49) |
| | (SE)/week 8 | | | | | | |
| | LS Mean | | −1.06 (2.6) | −0.68 (2.59) | −1.44 (2.64) | −2.57 (2.59) | −3.7 (2.11) |
| | Diff (SE)/ | | | | | | |
| | week 8 | | | | | | |
| Change | LS Mean | 1.05 (1.65) | −3.45 (2.34) | 2.29 (2.35) | −3.2 (2.4) | 1.93 (2.35) | −1.75 (1.65) |
| | (SE)/week 4 | | | | | | |
| | LS Mean | | −4.5 (2.87) | 1.24 (2.87) | −4.25 (2.92) | 0.88 (2.87) | −2.8 (2.33) |
| | Diff (SE)/ | | | | | | |
| | week 4 | | | | | | |
| | LS Mean | 0.69 (1.8) | 0.28 (2.56) | 0.48 (2.57) | −1.73 (2.62) | −1.97 (2.57) | −3.04 (1.81) |
| | (SE)/week 8 | | | | | | |
| | LS Mean | | −0.4 (3.14) | −0.21 (3.13) | −2.42 (3.19) | −2.66 (3.13) | −3.73 (2.55) |
| | Diff (SE)/ | | | | | | |
| | week 8 | | | | | | |
| Subcutaneous | LS Mean | −1.27 (1.02) | 0.05 (1.45) | −0.26 (1.47) | −1.67 (1.44) | −1.98 (1.44) | −1.76 (1.02) |
| Adipose Tissue | (SE)/week 4 | | | | | | |
| Volume (cm3)/ | LS Mean | | 1.32 (1.78) | 1.01 (1.82) | −0.4 (1.76) | −0.71 (1.76) | −0.49 (1.44) |
| % change | Diff (SE)/ | | | | | | |
| | week 4 | | | | | | |
| | LS Mean | 1.31 (1.21) | 1.01 (1.71) | 1.56 (1.75) | −3.73 (1.71) | −3.04 (1.7) | −1.45 (1.21) |
| | (SE)/week 8 | | | | | | |
| | LS Mean | | −0.31 (2.11) | 0.25 (2.15) | −5.04 (2.09)* | −4.36 (2.09)* | −2.77 (1.7) |
| | Diff (SE)/ | | | | | | |
| | week 8 | | | | | | |
| change | LS Mean | −3.94 (3.83) | 0.35 (5.42) | −0.74 (5.52) | −8.23 (5.39) | −3.02 (5.39) | −7.29 (3.82) |
| | (SE)/week 4 | | | | | | |
| | LS Mean | | 4.29 (6.68) | 3.19 (6.8) | −4.3 (6.6) | 0.92 (6.6) | −3.35 (5.39) |
| | Diff (SE)/ | | | | | | |
| | week 4 | | | | | | |
| | LS Mean | 4.74 (4.74) | 5.7 (6.7) | 5.53 (6.83) | −17.58 (6.67) | −8.75 (6.66) | −5.55 (4.73) |
| | (SE)/week 8 | | | | | | |
| | LS Mean | | 0.95 (8.26) | 0.79 (8.41) | −22.33 (8.16)* | −13.5 (8.17) | −10.3 (6.66) |
| | Diff (SE)/ | | | | | | |
| | week 8 | | | | | | |
| Sum of Fat | LS Mean | −0.8 (1.01) | −0.59 (1.44) | 1.89 (1.42) | 0.48 (1.42) | −2.87 (1.42) | −3.31 (1) |
| Mass of Arms | (SE)/week 4 | | | | | | |
| and Legs (kg)/ | LS Mean | | 0.21 (1.78) | 2.69 (1.74) | 1.28 (1.74) | −2.07 (1.74) | −2.51 (1.42) |
| % change | Diff (SE)/ | | | | | | |
| | week 4 | | | | | | |
| | LS Mean | −0.47 (1.34) | −2.44 (1.9) | 0.11 (1.87) | 0.27 (1.87) | −3.51 (1.87) | −5.2 (1.32) |
| | (SE)/week 8 | | | | | | |
| | LS Mean | | −1.97 (2.35) | 0.58 (2.31) | 0.74 (2.3) | −3.04 (2.3) | −4.73 (1.88)* |
| | Diff (SE)/ | | | | | | |
| | week 8 | | | | | | |
| change | LS Mean | −0.09 (0.12) | −0.06 (0.17) | 0.21 (0.16) | 0.01 (0.16) | −0.33 (0.16) | −0.41 (0.11) |
| | (SE)/week 4 | | | | | | |
| | LS Mean | | 0.02 (0.2) | 0.3 (0.2) | 0.09 (0.2) | −0.25 (0.2) | −0.33 (0.16) |
| | Diff (SE)/ | | | | | | |
| | week 4 | | | | | | |
| | LS Mean | −0.05 (0.15) | −0.26 (0.22) | −0.03 (0.21) | −0.07 (0.21) | −0.41 (0.21) | −0.62 (0.15) |
| | (SE)/week 8 | | | | | | |
| | LS Mean | | −0.21 (0.27) | 0.02 (0.26) | −0.02 (0.26) | −0.37 (0.26) | −0.58 (0.22)* |
| | Diff (SE)/ | | | | | | |
| | week 8 | | | | | | |
| Total Bone | LS Mean | 0.05 (0.25) | −0.22 (0.36) | −0.48 (0.35) | −0.04 (0.36) | 0.47 (0.36) | 0.55 (0.25) |
| Mineral Density | (SE)/week 4 | | | | | | |
| (BMD) Mass | LS Mean | | −0.27 (0.43) | −0.53 (0.43) | −0.09 (0.44) | 0.42 (0.44) | 0.5 (0.35) |
| (g/cm2)/% | Diff (SE)/ | | | | | | |
| change | week 4 | | | | | | |
| | LS Mean | −0.01 (0.28) | −0.47 (0.4) | −0.72 (0.4) | −0.08 (0.4) | −0.22 (0.41) | 0.16 (0.28) |
| | (SE)/week 8 | | | | | | |
| | LS Mean | | −0.46 (0.49) | −0.71 (0.49) | −0.07 (0.5) | −0.21 (0.5) | 0.17 (0.4) |
| | Diff (SE)/ | | | | | | |
| | week 8 | | | | | | |
| change | LS Mean | 0 (0.003) | −0.002 (0.004) | −0.005 (0.004) | 0 (0.004) | 0.005 (0.004) | 0.006 (0.003) |
| | (SE)/week 4 | | | | | | |
| | LS Mean | | 0 (0) | −0.01 (0) | 0 (0) | 0 (0) | 0.01 (0) |
| | Diff (SE)/ | | | | | | |
| | week 4 | | | | | | |
| | LS Mean | 0 (0.003) | −0.006 (0.004) | −0.008 (0.004) | 0 (0.004) | −0.002 (0.004) | 0.002 (0.003) |
| | (SE)/week 8 | | | | | | |
| | LS Mean | | −0.01 (0.01) | −0.01 (0.01) | 0 (0.01) | 0 (0.01) | 0 (0) |
| | Diff (SE)/ | | | | | | |
| | week 8 | | | | | | |
| Total Bone | LS Mean | 0.1 (0.28) | −0.16 (0.4) | −0.23 (0.4) | 0.25 (0.4) | −0.19 (0.4) | 0.62 (0.28) |
| Mineral Content | (SE)/week 4 | | | | | | |
| (BMC) Mass | LS Mean | | −0.26 (0.49) | −0.33 (0.49) | 0.15 (0.49) | −0.29 (0.49) | 0.52 (0.4) |
| (kg)/% change | Diff (SE)/ | | | | | | |
| | week 4 | | | | | | |
| | LS Mean | −0.42 (0.26) | −0.27 (0.36) | −0.48 (0.36) | −0.08 (0.36) | −0.56 (0.36) | 0.65 (0.25) |
| | (SE)/week 8 | | | | | | |
| | LS Mean | | 0.16 (0.45) | −0.05 (0.44) | 0.34 (0.44) | −0.14 (0.44) | 1.07 (0.36)* |
| | Diff (SE)/ | | | | | | |
| | week 8 | | | | | | |
| change | LS Mean | 0.002 (0.006) | −0.003 (0.009) | −0.005 (0.009) | 0.006 (0.009) | −0.003 (0.009) | 0.014 (0.006) |
| | (SE)/week 4 | | | | | | |
| | LS Mean | | −0.01 (0.01) | −0.01 (0.01) | 0 (0.01) | −0.01 (0.01) | 0.01 (0.01) |
| | Diff (SE)/ | | | | | | |
| | week 4 | | | | | | |
| | LS Mean | −0.009 (0.006) | −0.007 (0.008) | −0.011 (0.008) | −0.001 (0.008) | −0.012 (0.008) | 0.014 (0.006) |
| | (SE)/week 8 | | | | | | |
| | LS Mean | | 0 (0.01) | 0 (0.01) | 0.01 (0.01) | 0 (0.01) | 0.02 (0.01)* |
| | Diff (SE)/ | | | | | | |
| | week 8 | | | | | | |
| T-score for Total | LS Mean | −18.26 (18.01) | −51.19 (25.74) | −2.95 (25.45) | −6.66 (25.7) | −39.76 (25.87) | −0.9 (17.96) |
| Body/% change | (SE)/week 4 | | | | | | |
| | LS Mean | | −32.93 (31.23) | 15.31 (31.1) | 11.6 (31.56) | −21.5 (31.73) | 17.37 (25.43) |
| | Diff (SE)/ | | | | | | |
| | week 4 | | | | | | |
| | LS Mean | −20.87 (17.94) | −58.54 (25.64) | 3.83 (25.35) | 5.16 (25.6) | 23.12 (25.77) | 8.49 (17.89) |
| | (SE)/week 8 | | | | | | |
| | LS Mean | | −37.67 (31.11) | 24.71 (30.98) | 26.04 (31.44) | 44 (31.61) | 29.36 (25.33) |
| | Diff (SE)/ | | | | | | |
| | week 8 | | | | | | |
| change | LS Mean | 0.01 (0.03) | −0.03 (0.05) | −0.07 (0.05) | 0 (0.05) | 0.06 (0.05) | 0.08 (0.03) |
| | (SE)/week 4 | | | | | | |
| | LS Mean | | −0.04 (0.06) | −0.07 (0.06) | −0.01 (0.06) | 0.06 (0.06) | 0.07 (0.05) |
| | Diff (SE)/ | | | | | | |
| | week 4 | | | | | | |
| | LS Mean | 0 (0.04) | −0.07 (0.05) | −0.1 (0.05) | −0.01 (0.05) | −0.03 (0.06) | 0.03 (0.04) |
| | (SE)/week 8 | | | | | | |
| | LS Mean | | −0.07 (0.07) | −0.1 (0.07) | −0.01 (0.07) | −0.03 (0.07) | 0.03 (0.05) |
| | Diff (SE)/ | | | | | | |
| | week 8 | | | | | | |

Note:
Least-squares (LS) means, standard errors (SE) and p-value taken from ANCOVA. The model includes baseline measurement as covariate and the treatment as fixed factor.
*indicated p-value < 0.05;
**indicated p-value < 0.001.

Compared with placebo, R2477+R1033 combination significantly increased thigh muscle volume and total lean mass in medium and high dose groups (FIGS. 8 and 9), significantly increased appendicular lean body mass in all dose groups (FIG. 10) and significantly decreased total fat mass as well as android fat mass in the high dose group (Table 10). R2477+R1033, in the high dose group, significantly increased all of the major measures of muscle volume and lean mass compared with placebo, at both Week 4 and Week 8; the effects at Week 4 were generally less pronounced than at Week 8 (Table 10). At Week 8, the high dose anti-Activin A R2477+anti-GDF8 R1033 group exhibited increased % change in total bone mineral content from placebo, as measured by DXA, while total bone mineral density did not change (Table 10).

### Safety

All treatment emergent adverse events (TEAEs) were mild to moderate in severity except one severe TEAE of ‘radius fracture’ reported by a placebo subject. There were no serious adverse events, no deaths, and no discontinuations due to TEAEs. Headache was the most frequent TEAE in each of the treatment groups, occurring in 58.3% of all study subjects and in 50% of placebo subjects. Muscle spasms, nausea and mouth ulceration were the other frequent TEAEs in REGN2477+REGN1033 groups that occurred in 25% or more of subjects in the combination R2477+R1033 groups; these TEAEs occurred less frequently in the placebo group, but there does not appear to be any clear dose-response relationship. There were no clear signals of bleeding or diarrhea, adverse events that have been associated with blockade of activin receptors. One TEAE of a nosebleed (preferred term of epistaxis) occurred in a R2477+R1033 dose group-it resolved after 9 minutes.

Review of Potentially Clinically Significant Values (PCSVs) revealed no significant differences between REGN2477+REGN1033 and Placebo in Labs, Vital signs, and ECG that would indicate negative effects of REGN2477+REGN1033. Within laboratory, vital sign and ECG categories, there were 0-2 subjects with PCSVs in the combined REGN2477+REGN1033 dose groups (N of 24); however the percentage of subjects with PCSVs was equal to or lower than that found in the placebo group. There were no treatment-emergent PCSVs related to liver function tests.

### Conclusions

In healthy postmenopausal women, single intravenous doses of REGN2477+REGN1033 increased thigh muscle volume, total lean mass, and appendicular lean body mass. One surprising finding was the uniformity of the thigh muscle changes: all of the individuals exposed to the combination exhibited an increase in thigh muscle volume, as shown in FIG. 4. In addition, single intravenous doses of REGN2477+REGN1033 decreased total fat, and in particular, android fat mass. Treatment with REGN1033 alone increased thigh muscle volume.

In general, REGN2477, REGN1033, and REGN2477+REGN1033 in this clinical study were considered to have an acceptable safety profile and were well tolerated. There were no serious adverse events.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

What is claimed is:

1. A method for altering the body composition of a subject comprising administering a first composition comprising an effective amount of a GDF8 inhibitor and a second composition comprising an effective amount of an Activin A inhibitor to the subject.

2. The method of claim 1, wherein the altering of the body composition comprises inducing a reduction in fat mass in the subject comprising administering the first composition comprising an effective amount of a GDF8 inhibitor and the second composition comprising an effective amount of an Activin A inhibitor to the subject.

3. The method of claim 1, wherein the altering of the body composition comprises inducing an increase in muscle mass in the subject, the method comprising administering the first composition comprising an effective amount of the GDF8 inhibitor and the second composition comprising an effective amount of the Activin A inhibitor.

4. A method for altering the body composition of a subject comprising administering a composition comprising an effective amount of a GDF8 inhibitor and an effective amount of an Activin A inhibitor to the subject.

5. The method of claim 4, wherein the altering of the body composition comprises inducing a reduction in fat mass in a subject comprising administering the composition comprising an effective amount of the GDF8 inhibitor and an effective amount of the Activin A inhibitor to the subject.

6. The method of claim 3, wherein the altering of the body composition comprises-inducing an increase in muscle mass in the subject, the method comprising administering to the subject in need thereof the composition comprising an effective amount of the GDF8 inhibitor and an effective amount of the Activin A inhibitor.

7. The method of claim 1, wherein the effective amount of a GDF8 inhibitor comprises a dosing regimen selected from the group consisting of at least 0.01 mg/kg to about 10 gm/kg, 1 mg/kg to about 1 gm/kg, and 10 mg/kg to 100 mg/kg.

8. The method of claim 1, wherein the effective amount of an Activin A inhibitor comprises a dosing regimen selected from the group consisting of at least 0.01 mg/kg to about 10 gm/kg, 1 mg/kg to about 1 gm/kg, and 10 mg/kg to 100 mg/kg.

9. The method of claim 1, wherein the effective amount of a GDF8 inhibitor comprises a dosing regimen selected from a group consisting of a single dose of about 0.01 to about 20 mg/kg body weight, about 0.1 to about 10 mg/kg body weight, and about 0.1 to about 5 mg/kg body weight.

10. The method of claim 1, wherein the effective amount of an Activin A inhibitor comprises a dosing regimen selected from a group consisting of a single dose of about 0.01 to about 20 mg/kg body weight, about 0.1 to about 10 mg/kg body weight, and about 0.1 to about 5 mg/kg body weight.

11. The method of claim 7, wherein the effective amount of the GDF8 inhibitor is 6 mg/kg body weight of the subject.

12. The method of claim 7, wherein the effective amount of the Activin A inhibitor is 3 mg/kg or 10 mg/kg body weight of the subject.

13. The method of claim 1, wherein the first composition is formulated for intravenous, subcutaneous, or oral administration.

14. The method of claim 1, wherein the second composition is formulated for intravenous, subcutaneous, or oral administration.

15. The method of claim 1, wherein the first and second compositions are combined into a third composition prior to administration.

16. The method of claim 15, wherein the third composition is formulated for intravenous, subcutaneous, or oral administration.

17. The method of claim 3, wherein the composition is formulated for intravenous, subcutaneous, or oral administration.

18. The method of claim 1, further comprising measuring total fat mass and/or android fat mass in the subject before administration.

19. The method of claim 18, further comprising measuring total fat mass and/or android fat mass in the subject after administration, and administering the composition until the subject has a reduction in total fat mass and/or android fat mass of at least about 2% to 8%, at least about 2.5% to 6%, at least about 3% to 4%, or at least about 3.5%.

20. The method of claim 1, wherein the GDF8 inhibitor is an isolated antibody or an antigen-binding fragment thereof that specifically binds to GDF8.

21. The method of claim 20, wherein the antibody or antigen-binding fragment that specifically binds GDF8 comprises the heavy chain complementarity determining regions (HCDRs) of a heavy chain variable region (HCVR) comprising SEQ ID NO:360, and the light chain complementarity determining regions (LCDRs) of a light chain variable region (LCVR) comprising SEQ ID NO:368.

22. The method of claim 20, wherein the antibody or antigen-binding fragment that specifically binds GDF8 comprises three HCDRs comprising SEQ ID NO: 362, SEQ ID NO:364, and SEQ ID NO:366, and three LCDRs comprising SEQ ID NO:370, TTS, and SEQ ID NO:374.

23. The method of claim 1, wherein the Activin A inhibitor is an isolated antibody or antigen-binding fragment thereof that specifically binds Activin A.

24. The method of claim 23, wherein the antibody or antigen-binding fragment that specifically binds Activin A comprises the heavy chain complementarity determining regions (HCDRs) of a heavy chain variable region (HCVR) comprising SEQ ID NO:553, and the light chain complementarity determining regions (LCDRs) of a light chain variable region (LCVR) comprising SEQ ID NO:537.

25. The method of claim 23, wherein the antibody or antigen-binding fragment that specifically binds Activin A comprises three HCDRs comprising SEQ ID NO: 555, SEQ ID NO:557, and SEQ ID NO:559, and three LCDRs comprising SEQ ID NO:539, GAS, and SEQ ID NO:543.

26. The method of claim 1, wherein the Activin A inhibitor is in an amount selected from the group consisting of between 100% to 200% of the amount of the GDF8 inhibitor, between 100% and 250% of the amount of the GDF8 inhibitor, between 100% and 300% of the amount of the GDF8 inhibitor, and between 100% and 400% by weight of the amount of the GDF8 inhibitor.

27. The method of claim 26, wherein the amount of the Activin A inhibitor is about 1.5 to 2.0 times as large by weight as the amount of the GDF8 inhibitor.

28. The method of claim 1, wherein a reduction of fat mass in the subject is a reduction in total fat mass as measured by DXA (Dual-energy X-ray absorptiometry).

29. The method of claim 1, wherein a reduction of fat mass in the subject is a reduction in android fat mass as measured by DXA (Dual-energy X-ray absorptiometry).

30. The method of claim 1, wherein the subject experiences an increase in muscle volume after administration.

31. The method of claim 1, wherein the subject does not have a muscle wasting condition or disease.

32. A GDF8 inhibitor for use in a method for treating a disease or disorder characterized by increased fat mass, wherein the method comprises administrating to a subject the GDF8 inhibitor and an Activin A inhibitor.

33. An Activin A inhibitor for use in a method for treating a disease or disorder characterized by increased fat mass, wherein the method comprises administrating to a subject the Activin A inhibitor and a GDF8 inhibitor.

34. A non-therapeutic method for decreasing fat mass in a subject, the method comprising administering to the subject an Activin A inhibitor and a GDF8 inhibitor.


SEQUENCE LISTING (648 sequences)

SEQ ID NO: 1 - DNA, 366 bp, synthetic construct
  source 1..366: mol_type = other DNA
caggtgcagctggtgcagtctgggggaggcttggtccagcctggggggtccctgagactctcctgtgcagcctctggattcacctttaacacctatgccataagctgggtccgccaggctccagggaaggggctggaatgggtctcaactattactggtagtggttataacacatactacgcagactccgtgaagggccggttcaccatctccagagacaattccaagaacacactgtatctacagatgagcagcctgagagccgaggacacggccgtattttactgtgcgaaagactctcggtataactggaattacggaatttttgactactggggccagggaaccacggtcaccgtctcctca

SEQ ID NO: 2 - protein, 122 aa, synthetic construct
QVQLVQSGGGLVQPGGSLRLSCAASGFTFNTYAISWVRQAPGKGLEWVSTITGSGYNTYYADSVKGRFTISRDNSKNTLYLQMSSLRAEDTAVFYCAKDSRYNWNYGIFDYWGQGTTVTVSS

SEQ ID NO: 3 - DNA, 24 bp, synthetic construct
  source 1..24: mol_type = other DNA
ggattcacctttaacacctatgcc

SEQ ID NO: 4 - protein, 8 aa, synthetic construct
GFTFNTYA

SEQ ID NO: 5 - DNA, 24 bp, synthetic construct
  source 1..24: mol_type = other DNA
attactggtagtggttataacaca

SEQ ID NO: 6 - protein, 8 aa, synthetic construct
ITGSGYNT

SEQ ID NO: 7 - DNA, 45 bp, synthetic construct
  source 1..45: mol_type = other DNA
gcgaaagactctcggtataactggaattacggaatttttgactac

SEQ ID NO: 8 - protein, 15 aa, synthetic construct
AKDSRYNWNYGIFDY

SEQ ID NO: 9 - DNA, 324 bp, synthetic construct
  source 1..324: mol_type = other DNA
gacatccagatgacccagtctccagccaccctgtctgtgtctccaggggaaagagccaccctctcctgcagggccagtcagagtgttagcagcaacttagcctggtaccatcagaaacctggccaggctcccaggctcctcatctatggtgtatccaccagggccactggtatcccagccaggttcagtggcaatgggtctgggacagagttcactctcaccatcagcagcctgcagtctgaagattttgcagtttattactgtcagcagcataataactggccgctcactttcggcggagggaccaaggtggagatcaaacga

SEQ ID NO: 10 - protein, 108 aa, synthetic construct
DIQMTQSPATLSVSPGERATLSCRASQSVSSNLAWYHQKPGQAPRLLIYGVSTRATGIPARFSGNGSGTEFTLTISSLQSEDFAVYYCQQHNNWPLTFGGGTKVEIKR

SEQ ID NO: 11 - DNA, 18 bp, synthetic construct
  source 1..18: mol_type = other DNA
cagagtgttagcagcaac

SEQ ID NO: 12 - protein, 6 aa, synthetic construct
QSVSSN

SEQ ID NO: 13-14 - skipped (2 entries)

SEQ ID NO: 15 - DNA, 27 bp, synthetic construct
  source 1..27: mol_type = other DNA
cagcagcataataactggccgctcact

SEQ ID NO: 16 - protein, 9 aa, synthetic construct
QQHNNWPLT

SEQ ID NO: 17 - DNA, 375 bp, synthetic construct
  source 1..375: mol_type = other DNA
caggtgcagctggtgcagtcgggcccaggactggtgaagccttcggagaccctgtccctcacctgcactgtctctggtggctccatcagcaatagtaattactactggggctggatccgccagcccccagggaaggggctggagtggattgggactacctattatagtgggaccacctactacaacccgtccctcaagagtcgagtcaccatatccgtagacacgtccaagaaccagttctccctgaagctgagctctgtgaccgccgcagacacggctgtgtattactgtgcgagagattattatgatagtagtggttattattacaactggttcgatccctggggccagggaaccacggtcaccgtctcctca

SEQ ID NO: 18 - protein, 125 aa, synthetic construct
QVQLVQSGPGLVKPSETLSLTCTVSGGSISNSNYYWGWIRQPPGKGLEWIGTTYYSGTTYYNPSLKSRVTISVDTSKNQFSLKLSSVTAADTAVYYCARDYYDSSGYYYNWFDPWGQGTTVTVSS

SEQ ID NO: 19 - DNA, 30 bp, synthetic construct
  source 1..30: mol_type = other DNA
ggtggctccatcagcaatagtaattactac

SEQ ID NO: 20 - protein, 10 aa, synthetic construct
GGSISNSNYY

SEQ ID NO: 21 - DNA, 21 bp, synthetic construct
  source 1..21: mol_type = other DNA
acctattatagtgggaccacc

SEQ ID NO: 22 - protein, 7 aa, synthetic construct
TYYSGTT

SEQ ID NO: 23 - DNA, 51 bp, synthetic construct
  source 1..51: mol_type = other DNA
gcgagagattattatgatagtagtggttattattacaactggttcgatccc

SEQ ID NO: 24 - protein, 17 aa, synthetic construct
ARDYYDSSGYYYNWFDP

SEQ ID NO: 25 - DNA, 324 bp, synthetic construct
  source 1..324: mol_type = other DNA
gacatccagttgacccagtctccatcctccctgtctgcatctgtaggagacagagtcaccatcacttgccgggcaagtcaggacattagaaatgatttaggctggtatcagcagaaaccagggaaagcccctaagcgcctgatctatgctgcatccagtttgcaaagtggggtcccatcaaggttcagcggcagtggatctgggacagaattcagtctcacactcagcagcctgcagcctgaagattttgcaacttatttctgtctacagcatcatatttacccgtggacgttcggccaagggaccaagctggagatcaaacga

SEQ ID NO: 26 - protein, 108 aa, synthetic construct
DIQLTQSPSSLSASVGDRVTITCRASQDIRNDLGWYQQKPGKAPKRLIYAASSLQSGVPSRFSGSGSGTEFSLTLSSLQPEDFATYFCLQHHIYPWTFGQGTKLEIKR

SEQ ID NO: 27 - DNA, 18 bp, synthetic construct
  source 1..18: mol_type = other DNA
caggacattagaaatgat

SEQ ID NO: 28 - protein, 6 aa, synthetic construct
QDIRND

SEQ ID NO: 29-30 - skipped (2 entries)

SEQ ID NO: 31 - DNA, 27 bp, synthetic construct
  source 1..27: mol_type = other DNA
ctacagcatcatatttacccgtggacg

SEQ ID NO: 32 - protein, 9 aa, synthetic construct
LQHHIYPWT

SEQ ID NO: 33 - DNA, 375 bp, synthetic construct
  source 1..375: mol_type = other DNA
caagttcagctggtggagtcgggcccaggactggtgaagccttcggagaccctgtccctcacctgcactgtctatggtggctccatcagcagtggtaattactactggggctggatccgccagcccccagggaagggactggagtggattgggactatctattatagtggaagcgcctactacaacccgtccctcaagagtcgagtcaccatgtccgtagacacgtccaagaaccagttctccctgaaactgagctctgtgaccgccgcagacacggctgtttattactgtgtgagagattactatgatagtagtggtcattattacaactggttcgacccctggggccagggaaccacggtcaccgtctcctca

SEQ ID NO: 34 - protein, 125 aa, synthetic construct
QVQLVESGPGLVKPSETLSLTCTVYGGSISSGNYYWGWIRQPPGKGLEWIGTIYYSGSAYYNPSLKSRVTMSVDTSKNQFSLKLSSVTAADTAVYYCVRDYYDSSGHYYNWFDPWGQGTTVTVSS

SEQ ID NO: 35 - DNA, 30 bp, synthetic construct
  source 1..30: mol_type = other DNA
ggtggctccatcagcagtggtaattactac

SEQ ID NO: 36 - protein, 10 aa, synthetic construct
GGSISSGNYY

SEQ ID NO: 37 - DNA, 21 bp, synthetic construct
  source 1..21: mol_type = other DNA
atctattatagtggaagcgcc

SEQ ID NO: 38 - protein, 7 aa, synthetic construct
IYYSGSA

SEQ ID NO: 39 - DNA, 51 bp, synthetic construct
  source 1..51: mol_type = other DNA
gtgagagattactatgatagtagtggtcattattacaactggttcgacccc

SEQ ID NO: 40 - protein, 17 aa, synthetic construct
VRDYYDSSGHYYNWFDP

SEQ ID NO: 41 - DNA, 324 bp, synthetic construct
  source 1..324: mol_type = other DNA
gccatccagatgacccagtctccatcctccctgtctgcatctgtaggagacagagtcaccatcacttgccgggcaagtcaggacattagacatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatacttacccgtggacgttcggccaagggaccaaggtggagatcaaacga

SEQ ID NO: 42 - protein, 108 aa, synthetic construct
AIQMTQSPSSLSASVGDRVTITCRASQDIRHDLGWYQQKPGKAPKRLIYAASSLQSGVPSRFSGSGSGTEFTLTISSLQPEDFATYYCLQHNTYPWTFGQGTKVEIKR

SEQ ID NO: 43 - DNA, 18 bp, synthetic construct
  source 1..18: mol_type = other DNA
caggacattagacatgat

SEQ ID NO: 44 - protein, 6 aa, synthetic construct
QDIRHD

SEQ ID NO: 45-46 - skipped (2 entries)

SEQ ID NO: 47 - DNA, 27 bp, synthetic construct
  source 1..27: mol_type = other DNA
ctacagcataatacttacccgtggacg

SEQ ID NO: 48 - protein, 9 aa, synthetic construct
LQHNTYPWT

SEQ ID NO: 49 - DNA, 357 bp, synthetic construct
  source 1..357: mol_type = other DNA
caggtgcagctggtgcagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagagatcgtggatacacctttggggttgactactggggccagggaaccacggtcaccgtctcctca

SEQ ID NO: 50 - protein, 119 aa, synthetic construct
QVQLVQSGGGLVKPGGSLRLSCAASGFTFSSYSMNWVRQAPGKGLEWVSSISSSSSYIYYADSVKGRFTISRDNAKNSLYLQMNSLRAEDTAVYYCARDRGYTFGVDYWGQGTTVTVSS

SEQ ID NO: 51 - DNA, 24 bp, synthetic construct
  source 1..24: mol_type = other DNA
ggattcaccttcagtagctatagc

SEQ ID NO: 52 - protein, 8 aa, synthetic construct
GFTFSSYS

SEQ ID NO: 53 - DNA, 24 bp, synthetic construct
  source 1..24: mol_type = other DNA
attagtagtagtagtagttacata

SEQ ID NO: 54 - protein, 8 aa, synthetic construct
ISSSSSYI

SEQ ID NO: 55 - DNA, 36 bp, synthetic construct
  source 1..36: mol_type = other DNA
gcgagagatcgtggatacacctttggggttgactac

SEQ ID NO: 56 - protein, 12 aa, synthetic construct
ARDRGYTFGVDY

SEQ ID NO: 57 - DNA, 324 bp, synthetic construct
  source 1..324: mol_type = other DNA
gccatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatagttacccgtacacttttggccaggggaccaaggtggagatcaaacga

SEQ ID NO: 58 - protein, 108 aa, synthetic construct
AIQMTQSPSSLSASVGDRVTITCRASQGIRNDLGWYQQKPGKAPKRLIYAASSLQSGVPSRFSGSGSGTEFTLTISSLQPEDFATYYCLQHNSYPYTFGQGTKVEIKR

SEQ ID NO: 59 - DNA, 18 bp, synthetic construct
  source 1..18: mol_type = other DNA
cagggcattagaaatgat

SEQ ID NO: 60 - protein, 6 aa, synthetic construct
QGIRND

SEQ ID NO: 61-62 - skipped (2 entries)

SEQ ID NO: 63 - DNA, 27 bp, synthetic construct
  source 1..27: mol_type = other DNA
ctacagcataatagttacccgtacact

SEQ ID NO: 64 - protein, 9 aa, synthetic construct
LQHNSYPYT

SEQ ID NO: 65 - DNA, 375 bp, synthetic construct
  source 1..375: mol_type = other DNA
caggtgcagctggtgcagtcgggcccaggactggtgaagccttcggagaccctgtccctcacctgcactgtctctggtggctccatcatcacttatagttactactggggctggatccgccagcccccagggaaggggctggagtggattgggactatccatcatagtgggagcacctactacaacccgtccctcaagagtcgagtcaccatatccgtagacacgtccaagaaccagttctccctgacactgagttctgtgaccgccgcagacacggctgtgtattactgtgcgagagactactatgatagtagtggttattattataactggttcgacccctggggccagggaaccatggtcaccgtctcctca

SEQ ID NO: 66 - protein, 125 aa, synthetic construct
QVQLVQSGPGLVKPSETLSLTCTVSGGSIITYSYYWGWIRQPPGKGLEWIGTIHHSGSTYYNPSLKSRVTISVDTSKNQFSLTLSSVTAADTAVYYCARDYYDSSGYYYNWFDPWGQGTMVTVSS

SEQ ID NO: 67 - DNA, 30 bp, synthetic construct
  source 1..30: mol_type = other DNA
ggtggctccatcatcacttatagttactac

SEQ ID NO: 68 - protein, 10 aa, synthetic construct
GGSIITYSYY

SEQ ID NO: 69 - DNA, 21 bp, synthetic construct
  source 1..21: mol_type = other DNA
atccatcatagtgggagcacc

SEQ ID NO: 70 - protein, 7 aa, synthetic construct
IHHSGST

SEQ ID NO: 71 - DNA, 51 bp, synthetic construct
  source 1..51: mol_type = other DNA
gcgagagactactatgatagtagtggttattattataactggttcgacccc

SEQ ID NO: 72 - protein, 17 aa, synthetic construct
ARDYYDSSGYYYNWFDP

SEQ ID NO: 73 - DNA, 324 bp, synthetic construct
  source 1..324: mol_type = other DNA
gccatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatagttccccgtggacgttcggccaagggaccaaggtggagatcaaacga

SEQ ID NO: 74 - protein, 108 aa, synthetic construct
AIQMTQSPSSLSASVGDRVTITCRASQGIRNDLGWYQQKPGKAPKRLIYAASSLQSGVPSRFSGSGSGTEFTLTISSLQPEDFATYYCLQHNSSPWTFGQGTKVEIKR

SEQ ID NO: 75 - DNA, 18 bp, synthetic construct
  source 1..18: mol_type = other DNA
cagggcattagaaatgat

SEQ ID NO: 76 - protein, 6 aa, synthetic construct
QGIRND

SEQ ID NO: 77-78 - skipped (2 entries)

SEQ ID NO: 79 - DNA, 27 bp, synthetic construct
  source 1..27: mol_type = other DNA
ctacagcataatagttccccgtggacg

SEQ ID NO: 80 - protein, 9 aa, synthetic construct
LQHNSSPWT

SEQ ID NO: 81 - DNA, 369 bp, synthetic construct
  source 1..369: mol_type = other DNA
caggtgcacttgaaggagtctggtcctacgctggtgaaacccacacagaccctcacgctgacctgcaccttctctgggttctcactcagcactagtggagtgggtgtgggctggatccgtcagcccccaggaaaggccctggagtggcttgcactcatttattggaatgatgataagcgctacagcccatctctgaagagcaggctcaccatcaccaaggacacctccaaaaaccaggtggtccttacaatgaccaacatggaccctgtggacacagccacatattactgtacacacacctcccgttataactggcactacggcttccttgactactggggccagggaaccacggtcaccgtctcctca

SEQ ID NO: 82 - protein, 123 aa, synthetic construct
QVHLKESGPTLVKPTQTLTLTCTFSGFSLSTSGVGVGWIRQPPGKALEWLALIYWNDDKRYSPSLKSRLTITKDTSKNQVVLTMTNMDPVDTATYYCTHTSRYNWHYGFLDYWGQGTTVTVSS

SEQ ID NO: 83 - DNA, 30 bp, synthetic construct
  source 1..30: mol_type = other DNA
gggttctcactcagcactagtggagtgggt

SEQ ID NO: 84 - protein, 10 aa, synthetic construct
GFSLSTSGVG

SEQ ID NO: 85 - DNA, 21 bp, synthetic construct
  source 1..21: mol_type = other DNA
atttattggaatgatgataag

SEQ ID NO: 86 - protein, 7 aa, synthetic construct
IYWNDDK

SEQ ID NO: 87 - DNA, 45 bp, synthetic construct
  source 1..45: mol_type = other DNA
acacacacctcccgttataactggcactacggcttccttgactac

SEQ ID NO: 88 - protein, 15 aa, synthetic construct
THTSRYNWHYGFLDY

SEQ ID NO: 89 - DNA, 324 bp, synthetic construct
  source 1..324: mol_type = other DNA
gccatccagatgacccagtctccatcctcactgtctgcatctgtaggagacagagtcaccatcacttgtcgggcgagtcagggcattagcaattatttagcctggtttcagcagaaaccagggaaagcccctaagtccctgatctatgctgcatccagtttgcaaagtggggtcccatcaaagttcagcggcagtggatctgggacagatttcactctcaccatcagcagcctgcagcctgaagattttgcaacttattactgccaacaatataatagttacccgctcactttcggcggagggaccaaggtggaaatcaaacga

SEQ ID NO: 90 - protein, 108 aa, synthetic construct
AIQMTQSPSSLSASVGDRVTITCRASQGISNYLAWFQQKPGKAPKSLIYAASSLQSGVPSKFSGSGSGTDFTLTISSLQPEDFATYYCQQYNSYPLTFGGGTKVEIKR

SEQ ID NO: 91 - DNA, 18 bp, synthetic construct
  source 1..18: mol_type = other DNA
cagggcattagcaattat

SEQ ID NO: 92 - protein, 6 aa, synthetic construct
QGISNY

SEQ ID NO: 93-94 - skipped (2 entries)

SEQ ID NO: 95 - DNA, 27 bp, synthetic construct
  source 1..27: mol_type = other DNA
caacaatataatagttacccgctcact

SEQ ID NO: 96 - protein, 9 aa, synthetic construct
QQYNSYPLT

SEQ ID NO: 97 - DNA, 375 bp, synthetic construct
  source 1..375: mol_type = other DNA
gaggtgcagctggtgcagttgggcccaggactggtgaagccttcggagaccctgtccctcacctgcactgtctctggtggctccatcagcaatagtaattactactggggctggatccgccagcccccagggaaggggctggagtggattgggactacctattatagtgggaccacctactacaacccgtccctcaagagtcgagtcaccatatccgtagacacgtccaagaaccagttctccctgaagctgagctctgtgaccgccgcagacacggctgtgtattactgtgcgagagattattatgatagtagtggttattattacaactggttcgatccctggggccagggaaccacggtcaccgtctcctca

SEQ ID NO: 98 - protein, 125 aa, synthetic construct
EVQLVQLGPGLVKPSETLSLTCTVSGGSISNSNYYWGWIRQPPGKGLEWIGTTYYSGTTYYNPSLKSRVTISVDTSKNQFSLKLSSVTAADTAVYYCARDYYDSSGYYYNWFDPWGQGTTVTVSS

SEQ ID NO: 99 - DNA, 30 bp, synthetic construct; identical to SEQ ID NO: 19
  source 1..30: mol_type = other DNA

SEQ ID NO: 100 - protein, 10 aa, synthetic construct
GGSISNSNYY

SEQ ID NO: 101 - DNA, 21 bp, synthetic construct
  source 1..21: mol_type = other DNA
acctattatagtgggaccacc

SEQ ID NO: 102 - protein, 7 aa, synthetic construct
TYYSGTT

SEQ ID NO: 103 - DNA, 51 bp, synthetic construct; identical to SEQ ID NO: 23
  source 1..51: mol_type = other DNA

SEQ ID NO: 104 - protein, 17 aa, synthetic construct
ARDYYDSSGYYYNWFDP

SEQ ID NO: 105 - DNA, 324 bp, synthetic construct
  source 1..324: mol_type = other DNA
gccatccagatgacccagtctccatcctccctgtctgcatctgtaggagacagagtcaccatcacttgccgggcaagtcaggacattagaaatgatttaggctggtatcagcagaaaccagggaaagcccctaagcgcctgatctatgctgcatccagtttgcaaagtggggtcccatcaaggttcagcggcagtggatctgggacagaattcagtctcacactcagcagcctgcagcctgaagattttgcaacttatttctgtctacagcatcatatttacccgtggacgttcggccaagggaccaaggtggagatcaaacga

SEQ ID NO: 106 - protein, 108 aa, synthetic construct
AIQMTQSPSSLSASVGDRVTITCRASQDIRNDLGWYQQKPGKAPKRLIYAASSLQSGVPSRFSGSGSGTEFSLTLSSLQPEDFATYFCLQHHIYPWTFGQGTKVEIKR

SEQ ID NO: 107 - DNA, 18 bp, synthetic construct
  source 1..18: mol_type = other DNA
caggacattagaaatgat

SEQ ID NO: 108 - protein, 6 aa, synthetic construct
QDIRND

SEQ ID NO: 109-110 - skipped (2 entries)

SEQ ID NO: 111 - DNA, 27 bp, synthetic construct
  source 1..27: mol_type = other DNA
ctacagcatcatatttacccgtggacg

SEQ ID NO: 112 - protein, 9 aa, synthetic construct
LQHHIYPWT

SEQ ID NO: 113 - DNA, 355 bp, synthetic construct
  source 1..355: mol_type = other DNA
caggtgcagctggtggagtctgggggaggcgtggtccagcctgggaggtccctgagactctcctgtgcagcctctggattcaccttcagtcgctatggcattcactgggtccgccaggctccaggcaaggggctggagtgggtggctgttatatcttatgatggaagtgatgaatactatgtagactccgtgaagggccgattcagcatctcccgagacaattccaagaacacgctttatctacaaatgaacagtctgaggcctgcggactcggctgtttattactgtgtgaaaggagatctggaacttggttttgactactggggccagggaaccctggtcaccgtctcctcaa

SEQ ID NO: 114 - protein, 118 aa, synthetic construct
QVQLVESGGGVVQPGRSLRLSCAASGFTFSRYGIHWVRQAPGKGLEWVAVISYDGSDEYYVDSVKGRFSISRDNSKNTLYLQMNSLRPADSAVYYCVKGDLELGFDYWGQGTLVTVSS

SEQ ID NO: 115 - DNA, 24 bp, synthetic construct
  source 1..24: mol_type = other DNA
ggattcaccttcagtcgctatggc

SEQ ID NO: 116 - protein, 8 aa, synthetic construct
GFTFSRYG

SEQ ID NO: 117 - DNA, 24 bp, synthetic construct
  source 1..24: mol_type = other DNA
atatcttatgatggaagtgatgaa

SEQ ID NO: 118 - protein, 8 aa, synthetic construct
ISYDGSDE

SEQ ID NO: 119 - DNA, 33 bp, synthetic construct
  source 1..33: mol_type = other DNA
gtgaaaggagatctggaacttggttttgactac

SEQ ID NO: 120 - protein, 11 aa, synthetic construct
VKGDLELGFDY

SEQ ID NO: 121 - DNA, 337 bp, synthetic construct
  source 1..337: mol_type = other DNA
gatattgtgatgactcaggctgcaccctctatacctgtcattccaggagagtcagtatccatgtcctgcaggtctagtaagagtctcctgtacagtaatggacatacttacgtgtattggtttgtgcagaggccaggccagtctcctcagctcctgatatatcggatgtccaaccttgcctcaggagtcccagacaggttcagtggcagtgggtcaggaactgctttcacactgagaatcagtagagtggaggctgaggatgtgggtgtttattactgtatgcaaaatctagaatttccgctcacgttcggtgctgggaccaagctggagctgaaac

SEQ ID NO: 122 - protein, 112 aa, synthetic construct
DIVMTQAAPSIPVIPGESVSMSCRSSKSLLYSNGHTYVYWFVQRPGQSPQLLIYRMSNLASGVPDRFSGSGSGTAFTLRISRVEAEDVGVYYCMQNLEFPLTFGAGTKLELK

SEQ ID NO: 123 - DNA, 33 bp, synthetic construct
  source 1..33: mol_type = other DNA
aagagtctcctgtacagtaatggacatacttac

SEQ ID NO: 124 - protein, 11 aa, synthetic construct
KSLLYSNGHTY

SEQ ID NO: 125-126 - skipped (2 entries)

SEQ ID NO: 127 - DNA, 27 bp, synthetic construct
  source 1..27: mol_type = other DNA
atgcaaaatctagaatttccgctcacg

SEQ ID NO: 128 - protein, 9 aa, synthetic construct
MQNLEFPLT

SEQ ID NO: 129 - DNA, 355 bp, synthetic construct
  source 1..355: mol_type = other DNA
caggtgcagctggtggaggcggggggaggcgtggtccagcctgggaggtccctgagactctcctgtgcagcctctggattcaccttcagtcgctatggcattcactgggtccgccaggctccaggcaaggggctggagtgggtggctgttatatcttatgatggaactgatgaatactatgcagactccgtgaagggccgattcaccatctcccgagacaattccaagaacacgctttatctacaaatgaacagtctgagacctgcggactcggctgtatattactgtgcgaaaggagatctggaacttggttttgactactggggccagggaaccctggtcaccgtctcctcaa

SEQ ID NO: 130 - protein, 118 aa, synthetic construct
QVQLVEAGGGVVQPGRSLRLSCAASGFTFSRYGIHWVRQAPGKGLEWVAVISYDGTDEYYADSVKGRFTISRDNSKNTLYLQMNSLRPADSAVYYCAKGDLELGFDYWGQGTLVTVSS

SEQ ID NO: 131 - DNA, 24 bp, synthetic construct
  source 1..24: mol_type = other DNA
ggattcaccttcagtcgctatggc

SEQ ID NO: 132 - protein, 8 aa, synthetic construct
GFTFSRYG

SEQ ID NO: 133 - DNA, 24 bp, synthetic construct
  source 1..24: mol_type = other DNA
atatcttatgatggaactgatgaa

SEQ ID NO: 134 - protein, 8 aa, synthetic construct
ISYDGTDE

SEQ ID NO: 135 - DNA, 33 bp, synthetic construct
  source 1..33: mol_type = other DNA
gcgaaaggagatctggaacttggttttgactac

SEQ ID NO: 136 - protein, 11 aa, synthetic construct
AKGDLELGFDY

SEQ ID NO: 137 - DNA, 322 bp, synthetic construct
  source 1..322: mol_type = other DNA
gccatccggttgacccagtctccatcttccgtgtctgcatctgtgggagacagagtcaccatcacttgtcgggcgagtcaggatattagtatttggttagcctggtatcagcagagtccagggaaagcccctaaactcctgatcaatgttgcatcccgtttgcaaagtggggtcccatcaaggttcagcggcagtggatctgggacagatttcactctcaccatcaacggtctgcagcctgaagattttgtaacttactattgtcaacaggctaacagtttcccgatcaccttcggccaagggacacgactggcgaccaaac

SEQ ID NO: 138 - protein, 107 aa, synthetic construct
AIRLTQSPSSVSASVGDRVTITCRASQDISIWLAWYQQSPGKAPKLLINVASRLQSGVPSRFSGSGSGTDFTLTINGLQPEDFVTYYCQQANSFPITFGQGTRLATK

SEQ ID NO: 139 - DNA, 18 bp, synthetic construct
  source 1..18: mol_type = other DNA
caggatattagtatttgg

SEQ ID NO: 140 - protein, 6 aa, synthetic construct
QDISIW

SEQ ID NO: 141-142 - skipped (2 entries)

SEQ ID NO: 143 - DNA, 27 bp, synthetic construct
  source 1..27: mol_type = other DNA
caacaggctaacagtttcccgatcacc

SEQ ID NO: 144 - protein, 9 aa, synthetic construct
QQANSFPIT

SEQ ID NO: 145 - DNA, 358 bp, synthetic construct
  source 1..358: mol_type = other DNA
caggtgcagctggtgcagtctgggggaggcctggtcaagcctggggggtccctgagactctcctgtgcagcctctggattcatcttcaatacctataccatgaattgggtccgccaggctccagggaaggggctggagtgggtctcatccatcactagtcgtggtacttatatattctactcagactcacttaagggccgattcaccatttccagagacaacgccaataactcactgtttctgcaaatgaacagcctgagagtcgaagacacggctgtttattactgttcgagagatcgtggatacacctttggtcctgactactggggccagggaaccctggtcaccgtctcttcag

SEQ ID NO: 146 - protein, 119 aa, synthetic construct
QVQLVQSGGGLVKPGGSLRLSCAASGFIFNTYTMNWVRQAPGKGLEWVSSITSRGTYIFYSDSLKGRFTISRDNANNSLFLQMNSLRVEDTAVYYCSRDRGYTFGPDYWGQGTLVTVSS

SEQ ID NO: 147 - DNA, 24 bp, synthetic construct
  source 1..24: mol_type = other DNA
ggattcatcttcaatacctatacc

SEQ ID NO: 148 - protein, 8 aa, synthetic construct
GFIFNTYT

SEQ ID NO: 149 - DNA, 24 bp, synthetic construct
  source 1..24: mol_type = other DNA
atcactagtcgtggtacttatata

SEQ ID NO: 150 - protein, 8 aa, synthetic construct
ITSRGTYI

SEQ ID NO: 151 - DNA, 36 bp, synthetic construct
  source 1..36: mol_type = other DNA
tcgagagatcgtggatacacctttggtcctgactac

SEQ ID NO: 152 - protein, 12 aa, synthetic construct
SRDRGYTFGPDY

SEQ ID NO: 153 - DNA, 322 bp, synthetic construct
  source 1..322: mol_type = other DNA
gacatccagatgacccagtctccatcctccctgtctgcatctgtaggagacagagtcaccatcacttgccgggcaagtcaggacattagaaatgatttaggctggtatcagcagaaaccagggaaagcccctaagggcctgatctatgctgcatccagtttgcaaagtggggtcccatcaaggttcagcggcagtggatctgggacagaattcactctcacaatcagcagcctgcagcctgaagattttgcaacttatcactgtctacattatgattttcatcctcggacgttcggccaagggaccaaggtggaaatcaagc

SEQ ID NO: 154 - protein, 107 aa, synthetic construct
DIQMTQSPSSLSASVGDRVTITCRASQDIRNDLGWYQQKPGKAPKGLIYAASSLQSGVPSRFSGSGSGTEFTLTISSLQPEDFATYHCLHYDFHPRTFGQGTKVEIK

SEQ ID NO: 155 - DNA, 18 bp, synthetic construct
  source 1..18: mol_type = other DNA
caggacattagaaatgat

SEQ ID NO: 156 - protein, 6 aa, synthetic construct
QDIRND

SEQ ID NO: 157-158 - skipped (2 entries)

SEQ ID NO: 159 - DNA, 27 bp, synthetic construct
  source 1..27: mol_type = other DNA
ctacattatgattttcatcctcggacg

SEQ ID NO: 160 - protein, 9 aa, synthetic construct
LHYDFHPRT

SEQ ID NO: 161 - DNA, 391 bp, synthetic construct
  source 1..391: mol_type = other DNA
caggtgcaactggtggagtctgggggaggcgtggtccagcctgggaggtccctgagactctcctgtgcagcgtctggattcaccttcagtagctatgccatgcactgggtccgccaggctccaggcaaggggctggagtgggtggcagttatatggtatgatggaactaattattactatgcagactccgtgaagggccgattcaccatctccagagacaattccaagaacacgctgtatctgcaaatgaacagcctgagagccgaggacacggctgtgtattactgtgcgagagatcccctatattacgatattttgactggttattcccccgactactactacggtatggacgtctggggccaagggaccacggtcaccgtctcctcag

SEQ ID NO: 162 - protein, 130 aa, synthetic construct
QVQLVESGGGVVQPGRSLRLSCAASGFTFSSYAMHWVRQAPGKGLEWVAVIWYDGTNYYYADSVKGRFTISRDNSKNTLYLQMNSLRAEDTAVYYCARDPLYYDILTGYSPDYYYGMDVWGQGTTVTVSS

SEQ ID NO: 163 - DNA, 24 bp, synthetic construct
  source 1..24: mol_type = other DNA
ggattcaccttcagtagctatgcc

SEQ ID NO: 164 - protein, 8 aa, synthetic construct
GFTFSSYA

SEQ ID NO: 165 - DNA, 24 bp, synthetic construct
  source 1..24: mol_type = other DNA
atatggtatgatggaactaattat

SEQ ID NO: 166 - protein, 8 aa, synthetic construct
IWYDGTNY

SEQ ID NO: 167 - DNA, 69 bp, synthetic construct
  source 1..69: mol_type = other DNA
gcgagagatcccctatattacgatattttgactggttattcccccgactactactacggtatggacgtc

SEQ ID NO: 168 - protein, 23 aa, synthetic construct
ARDPLYYDILTGYSPDYYYGMDV

SEQ ID NO: 169 - DNA, 322 bp, synthetic construct
  source 1..322: mol_type = other DNA
gaaatagtgatgacgcagtctccagccaccctgtctgtgtctccaggggaaagagccaccctctcctgcagggccagtcagacttttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataagtggccgctcactttcggcggagggaccaaggtggagatcaaac

SEQ ID NO: 170 - protein, 107 aa, synthetic construct
EIVMTQSPATLSVSPGERATLSCRASQTFSSNLAWYQQKPGQAPRLLIYGASTRATGIPARFSGSGSGTEFTLTISSLQSEDFAVYYCQQYNKWPLTFGGGTKVEIK

SEQ ID NO: 171 - DNA, 18 bp, synthetic construct
  source 1..18: mol_type = other DNA
cagacttttagcagcaac

SEQ ID NO: 172 - protein, 6 aa, synthetic construct
QTFSSN

SEQ ID NO: 173-174 - skipped (2 entries)

SEQ ID NO: 175 - DNA, 27 bp, synthetic construct
  source 1..27: mol_type = other DNA
cagcagtataataagtggccgctcact

SEQ ID NO: 176 - protein, 9 aa, synthetic construct
QQYNKWPLT

SEQ ID NO: 177 - DNA, 367 bp, synthetic construct
  source 1..367: mol_type = other DNA
gaggtgcagctgttggagtctgggggaggcttggtccagcctggggagtccctgagactctcctgtgcagcctctggattcacctttagcagctatgccatgaactgggtccgccaggctccagggaaggggctggagtgggtctcaactatcagtggtagtggtggttatatatactacgcagactccgtgaagggccggttcaccatctccagagacaattccaagaacacgctgtatctgcaaatgaacagcctgagagccgaagacacggccgtatatttctgtgcgaaagattccaggtataactggaactacggcaattttgactactggggccagggaaccctggtcaccgtctcctcag

SEQ ID NO: 178 - protein, 122 aa, synthetic construct
EVQLLESGGGLVQPGESLRLSCAASGFTFSSYAMNWVRQAPGKGLEWVSTISGSGGYIYYADSVKGRFTISRDNSKNTLYLQMNSLRAEDTAVYFCAKDSRYNWNYGNFDYWGQGTLVTVSS

SEQ ID NO: 179 - DNA, 24 bp, synthetic construct
  source 1..24: mol_type = other DNA
ggattcacctttagcagctatgcc

SEQ ID NO: 180 - protein, 8 aa, synthetic construct
GFTFSSYA

SEQ ID NO: 181 - DNA, 24 bp, synthetic construct
  source 1..24: mol_type = other DNA
atcagtggtagtggtggttatata

SEQ ID NO: 182 - protein, 8 aa, synthetic construct
ISGSGGYI

SEQ ID NO: 183 - DNA, 45 bp, synthetic construct
  source 1..45: mol_type = other DNA
gcgaaagattccaggtataactggaactacggcaattttgactac

SEQ ID NO: 184 - protein, 15 aa, synthetic construct
AKDSRYNWNYGNFDY

SEQ ID NO: 185 - DNA, 322 bp, synthetic construct
  source 1..322: mol_type = other DNA
gaaatagtgatgacgcagtctccagccaccctgtctgtgtctccaggggaaagagccaccctctcctgcagggccagtcagaatgttagcagcaacttagcctggaacaagcagaaacctggccaggctcccagactcctcatctatgctacatccaccagggccactggtgtcccagccaggttcagtgccagtgggtctgggacagacttcgctctcaccatcaacagcctgcagtctgaagattttgcagtttattactgtcagcagtataataactggcctctcactttcggcggagggaccaaggtggagatcaaac

SEQ ID NO: 186 - protein, 107 aa, synthetic construct
EIVMTQSPATLSVSPGERATLSCRASQNVSSNLAWNKQKPGQAPRLLIYATSTRATGVPARFSASGSGTDFALTINSLQSEDFAVYYCQQYNNWPLTFGGGTKVEIK

SEQ ID NO: 187 - DNA, 18 bp, synthetic construct
  source 1..18: mol_type = other DNA
cagaatgttagcagcaac

SEQ ID NO: 188 - protein, 6 aa, synthetic construct
QNVSSN

SEQ ID NO: 189-190 - skipped (2 entries)

SEQ ID NO: 191 - DNA, 27 bp, synthetic construct
  source 1..27: mol_type = other DNA
cagcagtataataactggcctctcact

SEQ ID NO: 192 - protein, 9 aa, synthetic construct
QQYNNWPLT

SEQ ID NO: 193 - DNA, 367 bp, synthetic construct
  source 1..367: mol_type = other DNA
gaggtgcaactgttggaatctgggggaggcttggtacagcctggggggtccctgagactctcctgtgtagcctctcgattcaccttcagcagcaatgccatgagttgggtccgccaggctccagggacggggctggagtgggtctcagctattactggtagtggtagtaggacatactacgcagactccgtgaagggccggttcaccatctccagagacaattccaagaacacggtgtatctgcaaatgaacagcctgagaggcgaggacacggccgtatattactgtgcgaaagatcaagggggtacctggaactacggagattttgactactggggccagggaaccctggtcaccgtctcctcag

SEQ ID NO: 194 - protein, 122 aa, synthetic construct
EVQLLESGGGLVQPGGSLRLSCVASRFTFSSNAMSWVRQAPGTGLEWVSAITGSGSRTYYADSVKGRFTISRDNSKNTVYLQMNSLRGEDTAVYYCAKDQGGTWNYGDFDYWGQGTLVTVSS

SEQ ID NO: 195 - DNA, 24 bp, synthetic construct
  source 1..24: mol_type = other DNA
cgattcaccttcagcagcaatgcc

SEQ ID NO: 196 - protein, 8 aa, synthetic construct
RFTFSSNA

SEQ ID NO: 197 - DNA, 24 bp, synthetic construct
  source 1..24: mol_type = other DNA
attactggtagtggtagtaggaca

SEQ ID NO: 198 - protein, 8 aa, synthetic construct
ITGSGSRT

SEQ ID NO: 199 - DNA, 45 bp, synthetic construct
  source 1..45: mol_type = other DNA
gcgaaagatcaagggggtacctggaactacggagattttgactac

SEQ ID NO: 200 - protein, 15 aa, synthetic construct
AKDQGGTWNYGDFDY

SEQ ID NO: 201 - DNA, 322 bp, synthetic construct
  source 1..322: mol_type = other DNA
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtctcccagccaggttcagtggcagtgggtctgggacagacttcactctcaccatcagcagcctgcagtctgaagattttgcagtttattactgtcagcagtataataactggcctctcactttcggcggagggaccaaggtggagatcaagc

SEQ ID NO: 202 - protein, 107 aa, synthetic construct
EIVMTQSPATLSVSPGERATLSCRASQSVSSNLAWYQQKPGQAPRLLIYGASTRATGLPARFSGSGSGTDFTLTISSLQSEDFAVYYCQQYNNWPLTFGGGTKVEIK

SEQ ID NO: 203 - DNA, 18 bp, synthetic construct
  source 1..18: mol_type = other DNA
cagagtgttagcagcaac

SEQ ID NO: 204 - protein, 6 aa, synthetic construct
QSVSSN

SEQ ID NO: 205-206 - skipped (2 entries)

SEQ ID NO: 207 - DNA, 27 bp, synthetic construct
  source 1..27: mol_type = other DNA
cagcagtataataactggcctctcact

SEQ ID NO: 208 - protein, 9 aa, synthetic construct
QQYNNWPLT

SEQ ID NO: 209 - DNA, 373 bp, synthetic construct
  source 1..373: mol_type = other DNA
caggtgcagctggtggagtctgggggagacgtggtccagcctgggaggtccctgagactctcctgtacagcctctggattcaccttcagtagttatggcatgcactgggtccgccaggctccaggcagggggctggagtgggtggcagttatatcatttgatggaaaaaataaatactatgcagactccgtgaagggccgattcaccgtctccagagacaattccaagaacacgctgtttctgcaaatgaacagcctgagagctgaggacacggctctatattactgtgcgaaaaggatagcagcaactggttactactacttctacggtttggacgtctggggccaagggaccacggtcaccgtctcctcag

SEQ ID NO: 210 - protein, 124 aa, synthetic construct
QVQLVESGGDVVQPGRSLRLSCTASGFTFSSYGMHWVRQAPGRGLEWVAVISFDGKNKYYADSVKGRFTVSRDNSKNTLFLQMNSLRAEDTALYYCAKRIAATGYYYFYGLDVWGQGTTVTVSS

SEQ ID NO: 211 - DNA, 24 bp, synthetic construct
  source 1..24: mol_type = other DNA
ggattcaccttcagtagttatggc

SEQ ID NO: 212 - protein, 8 aa, synthetic construct
GFTFSSYG

SEQ ID NO: 213 - DNA, 24 bp, synthetic construct
  source 1..24: mol_type = other DNA
atatcatttgatggaaaaaataaa

SEQ ID NO: 214 - protein, 8 aa, synthetic construct
ISFDGKNK

SEQ ID NO: 215 - DNA, 51 bp, synthetic construct
  source 1..51: mol_type = other DNA
gcgaaaaggatagcagcaactggttactactacttctacggtttggacgtc

SEQ ID NO: 216 - protein, 17 aa, synthetic construct
AKRIAATGYYYFYGLDV

SEQ ID NO: 217 - DNA, 322 bp, synthetic construct
  source 1..322: mol_type = other DNA
gaaataatgatgacgcagtctccagccaccctgtctgtgtctccaggggaaagaggcaccctctcctgcagggccagtcagagtgttagtagcaacttagcctggtaccagcagaaacctggccaggctcccaggctcctcatctatggtgcatccaccagggccagtggtatcccagccaggttcagtggcagtgggtctgggacagagttcactctcaccgtcagcagcctgcagtctgaagattttgcagtttattactgtcagcagtataataactggccgctcactttcggcggagggaccaaggtggagatcaaac

SEQ ID NO: 218 - protein, 107 aa, synthetic construct
EIMMTQSPATLSVSPGERGTLSCRASQSVSSNLAWYQQKPGQAPRLLIYGASTRASGIPARFSGSGSGTEFTLTVSSLQSEDFAVYYCQQYNNWPLTFGGGTKVEIK

SEQ ID NO: 219 - DNA, 18 bp, synthetic construct
  source 1..18: mol_type = other DNA
cagagtgttagtagcaac

SEQ ID NO: 220 - protein, 6 aa, synthetic construct
QSVSSN

SEQ ID NO: 221-222 - skipped (2 entries)

SEQ ID NO: 223 - DNA, 27 bp, synthetic construct
  source 1..27: mol_type = other DNA
cagcagtataataactggccgctcact

SEQ ID NO: 224 - protein, 9 aa, synthetic construct
QQYNNWPLT

SEQ ID NO: 225 - DNA, 367 bp, synthetic construct
  source 1..367: mol_type = other DNA
gaggttcagctgttggagtctgggggaggcttggtacagccgggggggtccctgagactctcctgtgcagcctctggattcacctctatcacctatgccatgagctgggtccgccaggctccagggaaggggctggagtgggtctcagctattagtgttagtggtactaatacatactacgcagactccgtgaagggccggttcaccatctccagagacaagtccaagaacatgctgtatctgcaaatgaacagcctgagagccgaggacacggccgtgtattactgtgcgaaagatctcctacataactggaaatacgggacttttgatatctggggccaagggacaatggtcaccgtctcttcag

SEQ ID NO: 226 - protein, 122 aa, synthetic construct
EVQLLESGGGLVQPGGSLRLSCAASGFTSITYAMSWVRQAPGKGLEWVSAISVSGTNTYYADSVKGRFTISRDKSKNMLYLQMNSLRAEDTAVYYCAKDLLHNWKYGTFDIWGQGTMVTVSS

SEQ ID NO: 227 - DNA, 24 bp, synthetic construct
  source 1..24: mol_type = other DNA
ggattcacctctatcacctatgcc

SEQ ID NO: 228 - protein, 8 aa, synthetic construct
GFTSITYA

SEQ ID NO: 229 - DNA, 24 bp, synthetic construct
  source 1..24: mol_type = other DNA
attagtgttagtggtactaataca

SEQ ID NO: 230 - protein, 8 aa, synthetic construct
ISVSGTNT

SEQ ID NO: 231 - DNA, 45 bp, synthetic construct
  source 1..45: mol_type = other DNA
gcgaaagatctcctacataactggaaatacgggacttttgatatc

SEQ ID NO: 232 - protein, 15 aa, synthetic construct
AKDLLHNWKYGTFDI

SEQ ID NO: 233 - DNA, 322 bp, synthetic construct
  source 1..322: mol_type = other DNA
gaaatagtgatgacgcagtctccagccaccctgtctgtgtctccaggggaaagagccaccctctcctgcagggccagtcagagtgttgacagcaacttagtctggtaccaacaaaaacctggccaggttcccaggctcctcatctatggtgcatccaccagggccactggtatcccagccaggttcagtggcagtgggtctgggacagagttcactctcaccatcagcagcctgcagtctgaagattttgcagtttattactgtcagcagtataataagtggccgctcactttcggcggagggaccaaggtggagatcaaac

SEQ ID NO: 234 - protein, 107 aa, synthetic construct
EIVMTQSPATLSVSPGERATLSCRASQSVDSNLVWYQQKPGQVPRLLIYGASTRATGIPARFSGSGSGTEFTLTISSLQSEDFAVYYCQQYNKWPLTFGGGTKVEIK

SEQ ID NO: 235 - DNA, 18 bp, synthetic construct
  source 1..18: mol_type = other DNA
cagagtgttgacagcaac

SEQ ID NO: 236 - protein, 6 aa, synthetic construct
QSVDSN

SEQ ID NO: 237-238 - skipped (2 entries)

SEQ ID NO: 239 - DNA, 27 bp, synthetic construct
  source 1..27: mol_type = other DNA
cagcagtataataagtggccgctcact

SEQ ID NO: 240 - protein, 9 aa, synthetic construct
QQYNKWPLT

SEQ ID NO: 241 - DNA, 367 bp, synthetic construct; identical to SEQ ID NO: 225
  source 1..367: mol_type = other DNA

SEQ ID NO: 242 - protein, 122 aa, synthetic construct; identical to SEQ ID NO: 226

SEQ ID NO: 243 - DNA, 24 bp, synthetic construct
  source 1..24: mol_type = other DNA
ggattcacctctatcacctatgcc

SEQ ID NO: 244 - protein, 8 aa, synthetic construct
GFTSITYA

SEQ ID NO: 245 - DNA, 24 bp, synthetic construct
  source 1..24: mol_type = other DNA
attagtgttagtggtactaataca

SEQ ID NO: 246 - protein, 8 aa, synthetic construct
ISVSGTNT

SEQ ID NO: 247 - DNA, 45 bp, synthetic construct; identical to SEQ ID NO: 231
  source 1..45: mol_type = other DNA

SEQ ID NO: 248 - protein, 15 aa, synthetic construct
AKDLLHNWKYGTFDI

SEQ ID NO: 249 - DNA, 325 bp, synthetic construct
  source 1..325: mol_type = other DNA
gaaatagtgatgacgcagtctccagccaccctgtctgtgtctccaggggaaagagccaccctctcctgcagggccagtcagagtgttagcagcaacttagcctggaaccagcagaaacctggccaggctcccaggctcctcatctatggtgcatccaccagggccacgggtatcccagccaggttcagtggcagtgggtctgggacagagttcactctcaccatcagcagcctgcagtctgaagattttgcagtttattactgtcagcagtataacaactggcccatgtacacttttggccaggggaccaagctggagatcaaac

SEQ ID NO: 250 - protein, 108 aa, synthetic construct
EIVMTQSPATLSVSPGERATLSCRASQSVSSNLAWNQQKPGQAPRLLIYGASTRATGIPARFSGSGSGTEFTLTISSLQSEDFAVYYCQQYNNWPMYTFGQGTKLEIK

SEQ ID NO: 251 - DNA, 18 bp, synthetic construct
  source 1..18: mol_type = other DNA
cagagtgttagcagcaac

SEQ ID NO: 252 - protein, 6 aa, synthetic construct
QSVSSN

SEQ ID NO: 253-254 - skipped (2 entries)

SEQ ID NO: 255 - DNA, 30 bp, synthetic construct
  source 1..30: mol_type = other DNA
cagcagtataacaactggcccatgtacact

SEQ ID NO: 256 - protein, 10 aa, synthetic construct
QQYNNWPMYT

SEQ ID NO: 257 - DNA, 361 bp, synthetic construct
  source 1..361: mol_type = other DNA
caggtgcagctggtggagtctgggggaggcgtggtccagcctgggaggtccctgagactctcctgtgaagcctctggattcaccttcagtagttctggcatgcactgggtccgccaggctccaggcaaggggctgcagtgggtggctgttatatcatatgatggaaataataaattttatgaagactccgtgaagggccgattgaccatttccagagacaattccaacaacactctgtggctgcaaatgaacagcctgagagttgaagacacggctgtttattactgtgcgaaatcaggaggtagagtgggagccgcctttgcctactggggccagggaaccctggtcaccgtctcctcag

SEQ ID NO: 258 - protein, 120 aa, synthetic construct
QVQLVESGGGVVQPGRSLRLSCEASGFTFSSSGMHWVRQAPGKGLQWVAVISYDGNNKFYEDSVKGRLTISRDNSNNTLWLQMNSLRVEDTAVYYCAKSGGRVGAAFAYWGQGTLVTVSS

SEQ ID NO: 259 - DNA, 24 bp, synthetic construct
  source 1..24: mol_type = other DNA
ggattcaccttcagtagttctggc

SEQ ID NO: 260 - protein, 8 aa, synthetic construct
GFTFSSSG

SEQ ID NO: 261 - DNA, 24 bp, synthetic construct
  source 1..24: mol_type = other DNA
atatcatatgatggaaataataaa

SEQ ID NO: 262 - protein, 8 aa, synthetic construct
ISYDGNNK

SEQ ID NO: 263 - DNA, 39 bp, synthetic construct
  source 1..39: mol_type = other DNA
gcgaaatcaggaggtagagtgggagccgcctttgcctac

SEQ ID NO: 264 - protein, 13 aa, synthetic construct
AKSGGRVGAAFAY

SEQ ID NO: 265 - DNA, 337 bp, synthetic construct
  source 1..337: mol_type = other DNA
gatattgtgaacactcagtctccactctctctgcccgtcacccctggagagccggcctccatctcctgcaggtctagtcagagcctcctgtatggtaatggatacaactatttggattggtacctgcagaagccagggcactctccacagctcctgatctatttgggttctaatcggggctccggggtccctgacaggttcagtggcagtggatcaggcacagattttacactgaagatcagcagagtggaggctgaagatgttggcatttattactgcatgcaaactctacaaactccattcactttcggccctgggaccaaaatgtatatcaaac

SEQ ID NO: 266 - protein, 112 aa, synthetic construct
DIVNTQSPLSLPVTPGEPASISCRSSQSLLYGNGYNYLDWYLQKPGHSPQLLIYLGSNRGSGVPDRFSGSGSGTDFTLKISRVEAEDVGIYYCMQTLQTPFTFGPGTKMYIK

SEQ ID NO: 267 - DNA, 33 bp, synthetic construct
  source 1..33: mol_type = other DNA
cagagcctcctgtatggtaatggatacaactat

SEQ ID NO: 268 - protein, 11 aa, synthetic construct
QSLLYGNGYNY

SEQ ID NO: 269-270 - skipped (2 entries)

SEQ ID NO: 271 - DNA, 27 bp, synthetic construct
  source 1..27: mol_type = other DNA
atgcaaactctacaaactccattcact

SEQ ID NO: 272 - protein, 9 aa, synthetic construct
MQTLQTPFT

SEQ ID NO: 273 - DNA, 376 bp, synthetic construct
  source 1..376: mol_type = other DNA
gaggtgcagctgttggagtctgggggaggcttggtacagcctggggggtccctgagactctcctgtgcaggctctggaatcacctttagcagctatgccatgagctgggtccgccaggctccagggaaggggctggagtgggtctcagctattagtggtaatggtggtaccacaaactacgcagactccgtgaagggccggttcaccatctccagagacaactccaagaacacgctgtatctgcaaatgaacagcctgagagccgaggacacggccgtatattactgtgcgaaagaaagaatccttaccagcagctggacgaggtacggtattatggacgtctggggccaagggaccacggtcaccgtctcctcag

SEQ ID NO: 274 - protein, 125 aa, synthetic construct
EVQLLESGGGLVQPGGSLRLSCAGSGITFSSYAMSWVRQAPGKGLEWVSAISGNGGTTNYADSVKGRFTISRDNSKNTLYLQMNSLRAEDTAVYYCAKERILTSSWTRYGIMDVWGQGTTVTVSS

SEQ ID NO: 275 - DNA, 24 bp, synthetic construct
  source 1..24: mol_type = other DNA
ggaatcacctttagcagctatgcc

SEQ ID NO: 276 - protein, 8 aa, synthetic construct
GITFSSYA

SEQ ID NO: 277 - DNA, 24 bp, synthetic construct
  source 1..24: mol_type = other DNA
attagtggtaatggtggtaccaca

SEQ ID NO: 278 - protein, 8 aa, synthetic construct
ISGNGGTT

SEQ ID NO: 279 - DNA, 54 bp, synthetic construct
  source 1..54: mol_type = other DNA
gcgaaagaaagaatccttaccagcagctggacgaggtacggtattatggacgtc

SEQ ID NO: 280 - protein, 18 aa, synthetic construct
AKERILTSSWTRYGIMDV

SEQ ID NO: 281 - DNA, 322 bp, synthetic construct
  source 1..322: mol_type = other DNA
gaaatagtgatgacgcagtctccagccaccctgtctatgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggcctctcactttcggcggagggaccaagttagagatcaaac

SEQ ID NO: 282 - protein, 107 aa, synthetic construct
EIVMTQSPATLSMSPGERATLSCRASQSVSSNLAWYQQKPGQAPRLLIYGASTRATGIPARFSGSGSGTEFTLTISSLQSEDFAVYYCQQYNNWPLTFGGGTKLEIK

SEQ ID NO: 283 - DNA, 18 bp, synthetic construct
  source 1..18: mol_type = other DNA
cagagtgttagcagcaac

SEQ ID NO: 284 - protein, 6 aa, synthetic construct
QSVSSN

SEQ ID NO: 285-286 - skipped (2 entries)

SEQ ID NO: 287 - DNA, 27 bp, synthetic construct
  source 1..27: mol_type = other DNA
cagcagtataataactggcctctcact

SEQ ID NO: 288 - protein, 9 aa, synthetic construct
QQYNNWPLT

SEQ ID NO: 289 - DNA, 358 bp, synthetic construct
  source 1..358: mol_type = other DNA
caggtgcaactacagcagtggggcgcaggactgttgaagccttcggagaccctgtccctcacctgcgctgtctatggtgggtccttcagtggttactactggagctggatccgccagcccccagggaaggggctggagtggattggggaaatcaatcatagtggaaacaccaactacaacccgtccctcaagagtcgagtcaccatatcagtagacacgtccaagaaccagttctccctgaaactgaactctgtgaccgccgcggacacggctgtgtattactgtgcgagaagagaggctacagtaactccatactttgactactggggccagggaaccctggtcaccgtctcctcag

SEQ ID NO: 290 - protein, 119 aa, synthetic construct
QVQLQQWGAGLLKPSETLSLTCAVYGGSFSGYYWSWIRQPPGKGLEWIGEINHSGNTNYNPSLKSRVTISVDTSKNQFSLKLNSVTAADTAVYYCARREATVTPYFDYWGQGTLVTVSS

SEQ ID NO: 291 - DNA, 24 bp, synthetic construct
  source 1..24: mol_type = other DNA
ggtgggtccttcagtggttactac

SEQ ID NO: 292 - protein, 8 aa, synthetic construct
GGSGSGYY

SEQ ID NO: 293 - DNA, 21 bp, synthetic construct
  source 1..21: mol_type = other DNA
atcaatcatagtggaaacacc

SEQ ID NO: 294 - protein, 7 aa, synthetic construct
INHSGNT

SEQ ID NO: 295 - DNA, 39 bp, synthetic construct
  source 1..39: mol_type = other DNA
gcgagaagagaggctacagtaactccatactttgactac

SEQ ID NO: 296 - protein, 13 aa, synthetic construct
ARREATVTPYFDY

SEQ ID NO: 297 - DNA, 322 bp, synthetic construct
  source 1..322: mol_type = other DNA
gacatccagttgacccagtctccatccttcctgtctgcatctgtaggagacagagtcaccatcacttgctgggccagtcagggcattagcagttatttagcctggtatcagcaaaaaccagggaaagcccctaagctcctgatctatgctgcatccactttgcaaagtggggtcccatcaaggttcagcggcagtggatctgggacagaattcactctcacaatcagcagcctgcagcctgaagattttgcaacttattactgtcaacagcttaatagttatccgctcactttcggcggagggaccaaggtggagatcaaac

SEQ ID NO: 298 - protein, 107 aa, synthetic construct
DIQLTQSPSFLSASVGDRVTITCWASQGISSYLAWYQQKPGKAPKLLIYAASTLQSGVPSRFSGSGSGTEFTLTISSLQPEDFATYYCQQLNSYPLTFGGGTKVEIK

SEQ ID NO: 299 - DNA, 18 bp, synthetic construct
  source 1..18: mol_type = other DNA
cagggcattagcagttat

SEQ ID NO: 300 - protein, 6 aa, synthetic construct
QGISSY

SEQ ID NO: 301-302 - skipped (2 entries)

SEQ ID NO: 303 - DNA, 27 bp, synthetic construct
  source 1..27: mol_type = other DNA
caacagcttaatagttatccgctcact

SEQ ID NO: 304 - protein, 9 aa, synthetic construct
QQLNSYPLT

SEQ ID NO: 305 - DNA, 355 bp, synthetic construct
  source 1..355: mol_type = other DNA
caggtgcagctggtggagtctgggggaggcgtggtccagcctgggaggtccctgagactctcctgtgtagtctctggattcaacttcagtaggaatggcatacactgggtccgccaggctccaggcaaggggctggaatgggtggcagttatatcatatgatggaagaaataaattttatgtagagtccgtgaagggccgattcaccatctccagagacaattccaagaacacgctgtatctgcaaatgaacagtctgagagttgaggacacggctgtatattactgtgcgaaatcctcaattggagggttttttgaatactggggccagggaaccctggtcaccgtctcctcag

SEQ ID NO: 306 - protein, 118 aa, synthetic construct
QVQLVESGGGVVQPGRSLRLSCVVSGFNFSRNGIHWVRQAPGKGLEWVAVISYDGRNKFYVESVKGRFTISRDNSKNTLYLQMNSLRVEDTAVYYCAKSSIGGFFEYWGQGTLVTVSS

SEQ ID NO: 307 - DNA, 24 bp, synthetic construct
  source 1..24: mol_type = other DNA
ggattcaacttcagtaggaatggc

SEQ ID NO: 308 - protein, 8 aa, synthetic construct
GFNFSRNG

SEQ ID NO: 309 - DNA, 24 bp, synthetic construct
  source 1..24: mol_type = other DNA
atatcatatgatggaagaaataaa

SEQ ID NO: 310 - protein, 8 aa, synthetic construct
ISYDGRNK

SEQ ID NO: 311 - DNA, 33 bp, synthetic construct
  source 1..33: mol_type = other DNA
gcgaaatcctcaattggagggttttttgaatac

SEQ ID NO: 312 - protein, 11 aa, synthetic construct
AKSSIGGFFEY

SEQ ID NO: 313 - DNA, 337 bp, synthetic construct
  source 1..337: mol_type = other DNA
gatattgtgatgactcagtctccactctccctgcccgtcactcctggagagccggcctccatctcctgcaggtctagtcagagcctcctgcatagtaatggatacaactatttggattggtacctgcagaagccaggacagtctccacaactcatgatctatttgggttctcatcgggcctccggggtccctgacaggttcagtggcagtggatcaggcacagattttacactgaaaatcagcagagtggaggctgaggatgttggagtctattactgcattcaagttcaacaaactccgatcaccttcggccaagggacacggctggagattaaac

SEQ ID NO: 314 - protein, 112 aa, synthetic construct
DIVMTQSPLSLPVTPGEPASISCRSSQSLLHSNGYNYLDWYLQKPGQSPQLMIYLGSHRASGVPDRFSGSGSGTDFTLKISRVEAEDVGVYYCIQVQQTPITFGQGTRLEIK

SEQ ID NO: 315 - DNA, 33 bp, synthetic construct
  source 1..33: mol_type = other DNA
cagagcctcctgcatagtaatggatacaactat

SEQ ID NO: 316 - protein, 11 aa, synthetic construct
QSLLHSNGYNY

SEQ ID NO: 317-318 - skipped (2 entries)

SEQ ID NO: 319 - DNA, 27 bp, synthetic construct
  source 1..27: mol_type = other DNA
attcaagttcaacaaactccgatcacc

SEQ ID NO: 320 - protein, 9 aa, synthetic construct
IQVQQTPIT

SEQ ID NO: 321 - DNA, 337 bp, synthetic construct
  source 1..337: mol_type = other DNA
gatattgtgatgactcagactccactctcctcacctgtcacccttggacagccggcctccatctcctgcaggtctagtcaaagcctcgtacacagtgatggaaacacctacttgagttggcttcagcagaggccaggccagcctccaagactcctaatttacaagatttctaaccggttctctggggtcccagacagattcagtggcagtggggcagggacagatttcacactgaaaatcagcagggtggaagctgaggatgtcggggtttatttctgcatgcaagctacacaatttccgtacacttttggccaggggaccaagctggagatcaaag

SEQ ID NO: 322 - protein, 112 aa, synthetic construct
DIVMTQTPLSSPVTLGQPASISCRSSQSLVHSDGNTYLSWLQQRPGQPPRLLIYKISNRFSGVPDRFSGSGAGTDFTLKISRVEAEDVGVYFCMQATQFPYTFGQGTKLEIK

SEQ ID NO: 323 - DNA, 33 bp, synthetic construct
  source 1..33: mol_type = other DNA
caaagcctcgtacacagtgatggaaacacctac

SEQ ID NO: 324 - protein, 11 aa, synthetic construct
QSLVHSDGNTY

SEQ ID NO: 325-326 - skipped (2 entries)

SEQ ID NO: 327 - DNA, 27 bp, synthetic construct
  source 1..27: mol_type = other DNA
atgcaagctacacaatttccgtacact

SEQ ID NO: 328 - protein, 9 aa, synthetic construct
MQATQFPYT

SEQ ID NO: 329 - protein, 8 aa, synthetic construct
  VARIANT 6: note = Ala or Ser
  VARIANT 7: note = Phe or Tyr
  VARIANT 8: note = Gly or Ala
GFTFSXXX

SEQ ID NO: 330-331 - skipped (2 entries)

SEQ ID NO: 332 - protein, 6 aa, synthetic construct
  VARIANT 2: note = Asp or Gly
  VARIANT 6: note = Tyr or Trp
QXISBX

SEQ ID NO: 333 - skipped

SEQ ID NO: 334 - protein, 9 aa, synthetic construct
  VARIANT 2: note = Lys or Gln
  VARIANT 3: note = Ala or Tyr
  VARIANT 6: note = Ala or Phe
QXXBSXPLT

SEQ ID NO: 335 - protein, 330 aa, synthetic construct [sequence omitted]

SEQ ID NO: 336 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 337 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 338 - DNA, 1128 bp, Homo sapiens
  source 1..1128: mol_type = unassigned DNA
atgcaaaaactgcaactctgtgtttatatttacctgtttatgctgattgttgctggtccagtggatctaaatgagaacagtgagcaaaaagaaaatgtggaaaaagaggggctgtgtaatgcatgtacttggagacaaaacactaaatcttcaagaatagaagccattaagatacaaatcctcagtaaacttcgtctggaaacagctcctaacatcagcaaagatgttataagacaacttttacccaaagctcctccactccgggaactgattgatcagtatgatgtccagagggatgacagcagcgatggctctttggaagatgacgattatcacgctacaacggaaacaatcattaccatgcctacagagtctgattttctaatgcaagtggatggaaaacccaaatgttgcttctttaaatttagctctaaaatacaatacaataaagtagtaaaggcccaactatggatatatttgagacccgtcgagactcctacaacagtgtttgtgcaaatcctgagactcatcaaacctatgaaagacggtacaaggtatactggaatccgatctctgaaacttgacatgaacccaggcactggtatttggcagagcattgatgtgaagacagtgttgcaaaattggctcaaacaacctgaatccaacttaggcattgaaataaaagctttagatgagaatggtcatgatcttgctgtaaccttcccaggaccaggagaagatgggctgaatccgtttttagaggtcaaggtaacagacacaccaaaaagatccagaagggattttggtcttgactgtgatgagcactcaacagaatcacgatgctgtcgttaccctctaactgtggattttgaagcttttggatgggattggattatcgctcctaaaagatataaggccaattactgctctggagagtgtgaatttgtatttttacaaaaatatcctcatactcatctggtacaccaagcaaaccccagaggttcagcaggcccttgctgtactcccacaaagatgtctccaattaatatgctatattttaatggcaaagaacaaataatatatgggaaaattccagcgatggtagtagaccgctgtgggtgctcatga

SEQ ID NO: 339 - protein, 375 aa, Homo sapiens
MQKLQLCVYIYLFMLIVAGPVDLNENSEQKENVEKEGLCNACTWRQNTKSSRIEAIKIQILSKLRLETAPNISKDVIRQLLPKAPPLRELIDQYDVQRDDSSDGSLEDDDYHATTETIITMPTESDFLMQVDGKPKCCFFKFSSKIQYNKVVKAQLWIYLRPVETPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRRDFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 340 - protein, 109 aa, Homo sapiens
DFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 341 - DNA, 1224 bp, Homo sapiens
  source 1..1224: mol_type = unassigned DNA
atggtgctcgcggccccgctgctgctgggcttcctgctcctcgccctggagctgcggccccggggggaggcggccgagggccccgcggcggcggcggcggcggcggcggcggcggcagcggcgggggtcgggggggagcgctccagccggccagccccgtccgtggcgcccgagccggacggctgccccgtgtgcgtttggcggcagcacagccgcgagctgcgcctagagagcatcaagtcgcagatcttgagcaaactgcggctcaaggaggcgcccaacatcagccgcgaggtggtgaagcagctgctgcccaaggcgccgccgctgcagcagatcctggacctacacgacttccagggcgacgcgctgcagcccgaggacttcctggaggaggacgagtaccacgccaccaccgagaccgtcattagcatggcccaggagacggacccagcagtacagacagatggcagccctctctgctgccattttcacttcagccccaaggtgatgttcacaaaggtactgaaggcccagctgtgggtgtacctacggcctgtaccccgcccagccacagtctacctgcagatcttgcgactaaaacccctaactggggaagggaccgcagggggagggggcggaggccggcgtcacatccgtatccgctcactgaagattgagctgcactcacgctcaggccattggcagagcatcgacttcaagcaagtgctacacagctggttccgccagccacagagcaactggggcatcgagatcaacgcctttgatcccagtggcacagacctggctgtcacctccctggggccgggagccgaggggctgcatccattcatggagcttcgagtcctagagaacacaaaacgttcccggcggaacctgggtctggactgcgacgagcactcaagcgagtcccgctgctgccgatatcccctcacagtggactttgaggctttcggctgggactggatcatcgcacctaagcgctacaaggccaactactgctccggccagtgcgagtacatgttcatgcaaaaatatccgcatacccatttggtgcagcaggccaatccaagaggctctgctgggccctgttgtacccccaccaagatgtccccaatcaacatgctctacttcaatgacaagcagcagattatctacggcaagatccctggcatggtggtggatcgctgtggctgctcttaa

SEQ ID NO: 342 - protein, 407 aa, Homo sapiens
MVLAAPLLLGFLLLALELRPRGEAAEGPAAAAAAAAAAAAAGVGGERSSRPAPSVAPEPDGCPVCVWRQHSRELRLESIKSQILSKLRLKEAPNISREVVKQLLPKAPPLQQILDLHDFQGDALQPEDFLEEDEYHATTETVISMAQETDPAVQTDGSPLCCHFHFSPKVMFTKVLKAQLWVYLRPVPRPATVYLQILRLKPLTGEGTAGGGGGGRRHIRIRSLKIELHSRSGHWQSIDFKQVLHSWFRQPQSNWGIEINAFDPSGTDLAVTSLGPGAEGLHPFMELRVLENTKRSRRNLGLDCDEHSSESRCCRYPLTVDFEAFGWDWIIAPKRYKANYCSGQCEYMFMQKYPHTHLVQQANPRGSAGPCCTPTKMSPINMLYFNDKQQIIYGKIPGMVVDRCGCS

SEQ ID NO: 343 - protein, 109 aa, Homo sapiens
NLGLDCDEHSSESRCCRYPLTVDFEAFGWDWIIAPKRYKANYCSGQCEYMFMQKYPHTHLVQQANPRGSAGPCCTPTKMSPINMLYFNDKQQIIYGKIPGMVVDRCGCS

SEQ ID NO: 344 - DNA, 1503 bp, Homo sapiens
  source 1..1503: mol_type = unassigned DNA
atgagactccccaaactcctcactttcttgctttggtacctggcttggctggacctggaattcatctgcactgtgttgggtgcccctgacttgggccagagaccccaggggaccaggccaggattggccaaagcagaggccaaggagaggccccccctggcccggaacgtcttcaggccagggggtcacagctatggtgggggggccaccaatgccaatgccagggcaaagggaggcaccgggcagacaggaggcctgacacagcccaagaaggatgaacccaaaaagctgccccccagaccgggcggccctgaacccaagccaggacaccctccccaaacaaggcaggctacagcccggactgtgaccccaaaaggacagcttcccggaggcaaggcacccccaaaagcaggatctgtccccagctccttcctgctgaagaaggccagggagcccgggcccccacgagagcccaaggagccgtttcgcccaccccccatcacaccccacgagtacatgctctcgctgtacaggacgctgtccgatgctgacagaaagggaggcaacagcagcgtgaagttggaggctggcctggccaacaccatcaccagctttattgacaaagggcaagatgaccgaggtcccgtggtcaggaagcagaggtacgtgtttgacattagtgccctggagaaggatgggctgctgggggccgagctgcggatcttgcggaagaagccctcggacacggccaagccagcggcccccggaggcgggcgggctgcccagctgaagctgtccagctgccccagcggccggcagccggcctccttgctggatgtgcgctccgtgccaggcctggacggatctggctgggaggtgttcgacatctggaagctcttccgaaactttaagaactcggcccagctgtgcctggagctggaggcctgggaacggggcagggccgtggacctccgtggcctgggcttcgaccgcgccgcccggcaggtccacgagaaggccctgttcctggtgtttggccgcaccaagaaacgggacctgttctttaatgagattaaggcccgctctggccaggacgataagaccgtgtatgagtacctgttcagccagcggcgaaaacggcgggccccactggccactcgccagggcaagcgacccagcaagaaccttaaggctcgctgcagtcggaaggcactgcatgtcaacttcaaggacatgggctgggacgactggatcatcgcaccccttgagtacgaggctttccactgcgaggggctgtgcgagttcccattgcgctcccacctggagcccacgaatcatgcagtcatccagaccctgatgaactccatggaccccgagtccacaccacccacctgctgtgtgcccacgcggctgagtcccatcagcatcctcttcattgactctgccaacaacgtggtgtataagcagtatgaggacatggtcgtggagtcgtgtggctgcagg

SEQ ID NO: 345 - protein, 501 aa, Homo sapiens
MRLPKLLTFLLWYLAWLDLEFICTVLGAPDLGQRPQGTRPGLAKAEAKERPPLARNVFRPGGHSYGGGATNANARAKGGTGQTGGLTQPKKDEPKKLPPRPGGPEPKPGHPPQTRQATARTVTPKGQLPGGKAPPKAGSVPSSFLLKKAREPGPPREPKEPFRPPPITPHEYMLSLYRTLSDADRKGGNSSVKLEAGLANTITSFIDKGQDDRGPVVRKQRYVFDISALEKDGLLGAELRILRKKPSDTAKPAAPGGGRAAQLKLSSCPSGRQPASLLDVRSVPGLDGSGWEVFDIWKLFRNFKNSAQLCLELEAWERGRAVDLRGLGFDRAARQVHEKALFLVFGRTKKRDLFFNEIKARSGQDDKTVYEYLFSQRRKRRAPLATRQGKRPSKNLKARCSRKALHVNFKDMGWDDWIIAPLEYEAFHCEGLCEFPLRSHLEPTNHAVIQTLMNSMDPESTPPTCCVPTRLSPISILFIDSANNVVYKQYEDMVVESCGCR

SEQ ID NO: 346 - protein, 120 aa, Homo sapiens
APLATRQGKRPSKNLKARCSRKALHVNFKDMGWDDWIIAPLEYEAFHCEGLCEFPLRSHLEPTNHAVIQTLMNSMDPESTPPTCCVPTRLSPISILFIDSANNVVYKQYEDMVVESCGCR

SEQ ID NO: 347 - protein, 107 aa, synthetic construct
QAKHKQRKRLKSSCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 348 - protein, 109 aa, synthetic construct
ALDTNYCFSSTEKNCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 349 - protein, 111 aa, synthetic construct
DFGLDCDEHSTESRCCKRHPLYVDFSDVGWNDWIVAPPGYHAFYCSGECEFVFLQKYPHTHLVHQANPRGSAGPCCTPTKMSPINMLYFNGKEQIIYGKIPAMVVDRCGCS

SEQ ID NO: 350 - protein, 110 aa, synthetic construct
DFGLDCDEHSTESRCCVRQLYIDFRKDLGWKWIHEPKGYHANFCSGECEFVFLQKYPHTHLVHQANPRGSAGPCCTPTKMSPINMLYFNGKEQIIYGKIPAMVVDRCGCS

SEQ ID NO: 351 - protein, 114 aa, synthetic construct
DFGLDCDEHSTESRCCRYPLTVDFEAFGWDWIIAPKRYKANYCSGECPFPLADHLNSTNHAIVQTLVNSVNSKIPKACCTPTKMSPINMLYFNGKEQIIYGKIPAMVVDRCGCS

SEQ ID NO: 352 - protein, 112 aa, synthetic construct
DFGLDCDEHSTESRCCRYPLTVDFEAFGWDWIIAPKRYKANYCSGECPYIWSLDTQYSKVLALYNQHNPGASAAPCCTPTKMSPINMLYFNGKEQIIYGKIPAMVVDRCGCS

SEQ ID NO: 353 - protein, 109 aa, synthetic construct
DFGLDCDEHSTESRCCRYPLTVDFEAFGWDWIIAPKRYKANYCSGECEFVFLQKYPHTHLVHQANSVNSKIPCCTPTKMSPINMLYFNGKEQIIYGKIPAMVVDRCGCS

SEQ ID NO: 354 - protein, 109 aa, synthetic construct
DFGLDCDEHSTESRCCRYPLTVDFEAFGWDWIIAPKRYKANYCSGECEFVFLQKYPHTHLVHQANPGASAAPCCTPTKMSPINMLYFNGKEQIIYGKIPAMVVDRCGCS

SEQ ID NO: 355 - protein, 109 aa, synthetic construct
DFGLDCDEHSTESRCCRYPLTVDFEAFGWDWIIAPKRYKANYCSGECEFVFLQKYPHTHLVHQANPRGSAGPCCVPTELSAISMLYLDENEKVVLKNYQDMVVEGCGCS

SEQ ID NO: 356 - protein, 108 aa, synthetic construct
DFGLDCDEHSTESRCCRYPLTVDFEAFGWDWIIAPKRYKANYCSGECEFVFLQKYPHTHLVHQANPRGSAGPCCVPQALEPLPIVYYVGRKPKVEQLSNMIVRSCGCS

SEQ ID NO: 357 - protein, 109 aa, synthetic construct
DFGLDCDEHSTESRCCRYPLTVDFEAFGWDWIIAPKRYKANYCSGECEFVFLQKYPHTHLVHQANPRGSAGPCCTPTKMSPINMLYFNGKEKVVLKNYQDMVVEGCGCS

SEQ ID NO: 358 - protein, 108 aa, synthetic construct
DFGLDCDEHSTESRCCRYPLTVDFEAFGWDWIIAPKRYKANYCSGECEFVFLQKYPHTHLVHQANPRGSAGPCCTPTKMSPINMLYFNGKKPKVEQLSNMIVRSCGCS

SEQ ID NO: 359 - DNA, 360 bp, synthetic construct
  source 1..360: mol_type = other DNA
gaggtgcaggtgttggagtctgggggagacttggtacagcctggggggtccctgagactctcctgtgcagcctctggattcacctttagtgcctatgccatgacctgggtccgccaggctccagggaaggggctggagtgggtctcagctattagtggtagtggtggtagcgcatactacgcagactccgtgaagggccggttcaccatctccagagacaattccaagaacacggtatatctgcagatgaacagcctgagagccgaggacacggccgtatattactgtgcgaaagatggggcctggaaaatgtccggtttggacgtctggggccaagggaccacggtcatcgtctcctca

SEQ ID NO: 360 - protein, 120 aa, synthetic construct
EVQVLESGGDLVQPGGSLRLSCAASGFTFSAYAMTWVRQAPGKGLEWVSAISGSGGSAYYADSVKGRFTISRDNSKNTVYLQMNSLRAEDTAVYYCAKDGAWKMSGLDVWGQGTTVIVSS

SEQ ID NO: 361 - DNA, 24 bp, synthetic construct
  source 1..24: mol_type = other DNA
ggattcacctttagtgcctatgcc

SEQ ID NO: 362 - protein, 8 aa, synthetic construct
GFTFSAYA

SEQ ID NO: 363 - DNA, 24 bp, synthetic construct
  source 1..24: mol_type = other DNA
attagtggtagtggtggtagcgca

SEQ ID NO: 364 - protein, 8 aa, synthetic construct
ISGSGGSA

SEQ ID NO: 365 - DNA, 39 bp, synthetic construct
  source 1..39: mol_type = other DNA
gcgaaagatggggcctggaaaatgtccggtttggacgtc

SEQ ID NO: 366 - protein, 13 aa, synthetic construct
AKDGAWKMSGLDV

SEQ ID NO: 367 - DNA, 321 bp, synthetic construct
  source 1..321: mol_type = other DNA
gacatccagatgacccagtctccagcctccctgtctgcatctgttggagacagagtcaccatcacttgtcgggcgagtcaggacattagcgattatttagcctggtatcagcagaaaccagggaaaattcctaggctcctgatctatactacatccactttgcaatcaggggtcccatctcggttccgtggcagtgggtctgggacagatttcactctcaccatcagcagcctgcagcctgaagatgttgcaacttattactgtcagaagtatgacagtgccccgctcactttcggcggagggaccaaggtggagatcaaa

SEQ ID NO: 368 - protein, 107 aa, synthetic construct
DIQMTQSPASLSASVGDRVTITCRASQDISDYLAWYQQKPGKIPRLLIYTTSTLQSGVPSRFRGSGSGTDFTLTISSLQPEDVATYYCQKYDSAPLTFGGGTKVEIK

SEQ ID NO: 369 - DNA, 18 bp, synthetic construct
  source 1..18: mol_type = other DNA
caggacattagcgattat

SEQ ID NO: 370 - protein, 6 aa, synthetic construct
QDISDY

SEQ ID NO: 371-372 - skipped (2 entries)

SEQ ID NO: 373 - DNA, 27 bp, synthetic construct
  source 1..27: mol_type = other DNA
cagaagtatgacagtgccccgctcact

SEQ ID NO: 374 - protein, 9 aa, synthetic construct
QKYDSAPLT

SEQ ID NO: 375 - DNA, 363 bp, synthetic construct
  source 1..363: mol_type = other DNA
caggtgcagctggtggagtctgggggaggcgtggtccagcctgggaagtccctgcgactctcctgtgcagcgtctggattcaccttcagtagttttggcatgcattgggtccgccaggctccaggcaaggggctggagtgggtggcagttattgggtatgatggaggtaatgaatactatgccgactccgtgaagggccgattcaccatctccagagacaattccaagaacacgctgaatctgcaaatgagcagcctgagagccgaagacacggctgtgtattattgttcgactataagtcattacgatattttgagcggtatggacgtctggggccgagggaccacggtcaccgtctcctca

SEQ ID NO: 376 - protein, 121 aa, synthetic construct
QVQLVESGGGVVQPGKSLRLSCAASGFTFSSFGMHWVRQAPGKGLEWVAVIGYDGGNEYYADSVKGRFTISRDNSKNTLNLQMSSLRAEDTAVYYCSTISHYDILSGMDVWGRGTTVTVSS

SEQ ID NO: 377 - DNA, 24 bp, synthetic construct
  source 1..24: mol_type = other DNA
ggattcaccttcagtagttttggc

SEQ ID NO: 378 - protein, 8 aa, synthetic construct
GFTFSSFG

SEQ ID NO: 379 - DNA, 24 bp, synthetic construct
  source 1..24: mol_type = other DNA
attgggtatgatggaggtaatgaa

SEQ ID NO: 380 - protein, 8 aa, synthetic construct
IGYDGGNE

SEQ ID NO: 381 - DNA, 42 bp, synthetic construct
  source 1..42: mol_type = other DNA
tcgactataagtcattacgatattttgagcggtatggacgtc

SEQ ID NO: 382 - protein, 14 aa, synthetic construct
STISHYDILSGMDV

SEQ ID NO: 383 - DNA, 324 bp, synthetic construct
  source 1..324: mol_type = other DNA
gacatccagatgacccagtctccatcttccgtgtctgcatctgtaggagacagagtcaccatcacttgtcgggcgagtcagggtattagcaactggttagcctggtatcagcagaaaccagggaaagcccctaagctcctgatctttgctgcatccagtttgcaaagtggggtcccatcacggttcagcggcagtgcatctgggacagatttcactctcaccatcaacagcctgcagcctgaagattttgcaacttactattgtcaacaggctaacagtttcccgctcactttcggcggagggaccaaggtggagatcaaacga

SEQ ID NO: 384 - protein, 108 aa, synthetic construct
DIQMTQSPSSVSASVGDRVTITCRASQGISNWLAWYQQKPGKAPKLLIFAASSLQSGVPSRFSGSASGTDFTLTINSLQPEDFATYYCQQANSFPLTFGGGTKVEIKR

SEQ ID NO: 385 - DNA, 18 bp, synthetic construct
  source 1..18: mol_type = other DNA
cagggtattagcaactgg

SEQ ID NO: 386 - protein, 6 aa, synthetic construct
QGISNW

SEQ ID NO: 387-388 - skipped (2 entries)

SEQ ID NO: 389 - DNA, 27 bp, synthetic construct
  source 1..27: mol_type = other DNA
caacaggctaacagtttcccgctcact

SEQ ID NO: 390 - protein, 9 aa, synthetic construct
QQANSFPLT

SEQ ID NO: 391 - protein, 340 aa, synthetic construct
EAETRECIYYNANWELERTNQSGLERCEGEQDKRLHCYASWANSSGTIELVKKGCWLDDFNCYDRQECVATEENPQVYFCCCEGNFCNERFTHLPEAGGPEVTYEPPPTAPSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 392 - DNA, 366 bp, synthetic construct
  source 1..366: mol_type = other DNA
caggtacagctgcagcagtcaggtccaggactgctgaagccctcgcagaccctctcactcacctgtgccatctccggggacagtgtctctagcaacagtgctgcttggagttggatcaggcagtccccatcgagaggccttgagtggctgggaaggacatattacagggccaactggtttaatgattatgcactttctgtgaaaagtcgaataaccatcaacccagtcacatccacgaaccacttctccctgcagctgcactctgtgactcccgaggacacggctgtgtattactgtgcaagagaaggggctctgggatactactttgactcctggggccagggaaccctggtcaccgtctcctca

SEQ ID NO: 393 - protein, 122 aa, synthetic construct
QVQLQQSGPGLLKPSQTLSLTCAISGDSVSSNSAAWSWIRQSPSRGLEWLGRTYYRANWFNDYALSVKSRITINPVTSTNHFSLQLHSVTPEDTAVYYCAREGALGYYFDSWGQGTLVTVSS

SEQ ID NO: 394 - DNA, 30 bp, synthetic construct
  source 1..30: mol_type = other DNA
ggggacagtgtctctagcaacagtgctgct

SEQ ID NO: 395 - protein, 10 aa, synthetic construct
GDSVSSNSAA

SEQ ID NO: 396 - DNA, 27 bp, synthetic construct
  source 1..27: mol_type = other DNA
acatattacagggccaactggtttaat

SEQ ID NO: 397 - protein, 9 aa, synthetic construct
TYYRANWFN

SEQ ID NO: 398 - DNA, 36 bp, synthetic construct
  source 1..36: mol_type = other DNA
gcaagagaaggggctctgggatactactttgactcc

SEQ ID NO: 399 - protein, 12 aa, synthetic construct
AREGALGYYFDS

SEQ ID NO: 400 - DNA, 339 bp, synthetic construct
  source 1..339: mol_type = other DNA
gacatcgtgatgacccagtctccagactccctggctgtgtctctgggcgagagggccaccatcaactgcaagtccagtcaaagtgttttatacagctccaacaataagaattatttagcttggtaccaacagaaaccagggcagcctcctacactgctcatttactgggcatctacccgggaatccggggtccctgaccgattcagtggcagcgggtctgggacagatttcactctcaccatcagcagcctgcaggcggaagatgtggcaatttattactgtcaccaatattttattactccactcactttcggcggagggaccaaggtggagatcaaa

SEQ ID NO: 401 - protein, 113 aa, synthetic construct
DIVMTQSPDSLAVSLGERATINCKSSQSVLYSSNNKNYLAWYQQKPGQPPTLLIYWASTRESGVPDRFSGSGSGTDFTLTISSLQAEDVAIYYCHQYFITPLTFGGGTKVEIK

SEQ ID NO: 402 - DNA, 36 bp, synthetic construct
  source 1..36: mol_type = other DNA
caaagtgttttatacagctccaacaataagaattat

SEQ ID NO: 403 - protein, 12 aa, synthetic construct
QSVLYSSNNKNY

SEQ ID NO: 404-405 - skipped (2 entries)

SEQ ID NO: 406 - DNA, 27 bp, synthetic construct
  source 1..27: mol_type = other DNA
caccaatattttattactccactcact

SEQ ID NO: 407 - protein, 9 aa, synthetic construct
HQYFITPLT

SEQ ID NO: 408 - DNA, 378 bp, synthetic construct
  source 1..378: mol_type = other DNA
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaatacaatgcagactccgtgaagggccgattcaccatctccagagacaattccaagaacacggtgtatctgcaaatgaacagcctgagagccgaggacacggctgtgtattactgtgcgagagcccggaattacgatattttgactggttattataacctcggtatggacgtctggggccaagggaccacggtcaccgtctcctca

SEQ ID NO: 409 - protein, 126 aa, synthetic construct
QVQLVESGGGVVQPGRSLRLSCAASGFTFSSYGMHWVRQAPGKGLEWVAVIWYDGSNKYNADSVKGRFTISRDNSKNTVYLQMNSLRAEDTAVYYCARARNYDILTGYYNLGMDVWGQGTTVTVSS

SEQ ID NO: 410 - DNA, 24 bp, synthetic construct
  source 1..24: mol_type = other DNA
ggattcaccttcagtagctatggc

SEQ ID NO: 411 - protein, 8 aa, synthetic construct
GFTFSSYG

SEQ ID NO: 412 - DNA, 24 bp, synthetic construct
  source 1..24: mol_type = other DNA
atatggtatgatggaagtaataaa

SEQ ID NO: 413 - protein, 8 aa, synthetic construct
IWYDGSNK

SEQ ID NO: 414 - DNA, 57 bp, synthetic construct
  source 1..57: mol_type = other DNA
gcgagagcccggaattacgatattttgactggttattataacctcggtatggacgtc

SEQ ID NO: 415 - protein, 23 aa, synthetic construct
ARARNYDILTGYYNLGMDVSPGK

SEQ ID NO: 416 - DNA, 321 bp, synthetic construct
  source 1..321: mol_type = other DNA
gacatccagatgacccagtctccatcctccctgtctgcatctgtaggagacagagtcaccatcacttgccgggcaagtcagggcattagaaatgatttaggctggtatcaacagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatagttacccgtacacttttggccaggggaccaagctggagatcaaa

SEQ ID NO: 417 - protein, 107 aa, synthetic construct
DIQMTQSPSSLSASVGDRVTITCRASQGIRNDLGWYQQKPGKAPKRLIYAASSLQSGVPSRFSGSGSGTEFTLTISSLQPEDFATYYCLQHNSYPYTFGQGTKLEIK

SEQ ID NO: 418 - DNA, 18 bp, synthetic construct
  source 1..18: mol_type = other DNA
cagggcattagaaatgat

SEQ ID NO: 419 - protein, 6 aa, synthetic construct
QGIRND

SEQ ID NO: 420-421 - skipped (2 entries)

SEQ ID NO: 422 - DNA, 27 bp, synthetic construct
  source 1..27: mol_type = other DNA
ctacagcataatagttacccgtacact

SEQ ID NO: 423 - protein, 9 aa, synthetic construct
LQHNSYPYT

SEQ ID NO: 424 - DNA, 375 bp, synthetic construct
  source 1..375: mol_type = other DNA
gaagtgcagctggtggagtctgggggaaacttggtacagtctggcaggtccctgagactctcctgtacagcctctggattcgcctttgatgattttgccatgcactgggtccggcaagttccagggaagggcctggagtgggtctcaggtattagttggaatagtgataccatcggctatgcggactctgtgaagggccgattcaccatttccagagacaacgcccagaactccctgtttctgcaaatggacagtctgagagctgaggacacggccttgtattactgtgtaaaagatatggttcggggacttataggctactactactacggtatggacgtctggggccaagggaccacggtcaccgtctcctca

SEQ ID NO: 425 - protein, 125 aa, synthetic construct
EVQLVESGGNLVQSGRSLRLSCTASGFAFDDFAMHWVRQVPGKGLEWVSGISWNSDTIGYADSVKGRFTISRDNAQNSLFLQMDSLRAEDTALYYCVKDMVRGLIGYYYYGMDVWGQGTTVTVSS

SEQ ID NO: 426 - DNA, 24 bp, synthetic construct
  source 1..24: mol_type = other DNA
ggattcgcctttgatgattttgcc

SEQ ID NO: 427 - protein, 8 aa, synthetic construct
GFAFDDFA

SEQ ID NO: 428 - DNA, 24 bp, synthetic construct
  source 1..24: mol_type = other DNA
attagttggaatagtgataccatc

SEQ ID NO: 429 - protein, 8 aa, synthetic construct
ISWNSDTI

SEQ ID NO: 430 - DNA, 54 bp, synthetic construct
  source 1..54: mol_type = other DNA
gtaaaagatatggttcggggacttataggctactactactacggtatggacgtc

SEQ ID NO: 431 - protein, 18 aa, synthetic construct
VKDMVRGLIGYYYYGMDV

SEQ ID NO: 432 - DNA, 321 bp, synthetic construct
  source 1..321: mol_type = other DNA
gaaatagtgttgacgcagtctccagccatcctgtctttgtctccaggggaaagagccatcctctcctgcagggccagtcagagtatttacacctacttatcctggtaccaacagacacctggccgggctcccaggctcctcatctatgagacatccagcagggccactggcatcccagccaggttcattggcagtgggtctgggacagacttcactctcaccatcagtagcctagagcctgaagattttgcattttattactgtcagcaccgtagcgactggcctcccacttttggccaggggaccaagctggagatcaaa

SEQ ID NO: 433 - protein, 107 aa, synthetic construct
EIVLTQSPAILSLSPGERAILSCRASQSIYTYLSWYQQTPGRAPRLLIYETSSRATGIPARFIGSGSGTDFTLTISSLEPEDFAFYYCQHRSDWPPTFGQGTKLEIK

SEQ ID NO: 434 - DNA, 18 bp, synthetic construct
  source 1..18: mol_type = other DNA
cagagtatttacacctac

SEQ ID NO: 435 - protein, 6 aa, synthetic construct
QSIYTY

SEQ ID NO: 436-437 - skipped (2 entries)

SEQ ID NO: 438 - DNA, 27 bp, synthetic construct
  source 1..27: mol_type = other DNA
cagcaccgtagcgactggcctcccact

SEQ ID NO: 439 - protein, 9 aa, synthetic construct
QHRSDWPPT

SEQ ID NO: 440 - DNA, 351 bp, synthetic construct
  source 1..351: mol_type = other DNA
caggtgcagctggtggagtcggggggaggcgtggtccagcctgggaggtccctgagactctcctgtgtagcgtctggattcaccgtcagtagttatggcattcactgggtccgccaggctccaggcaagggactggagtgggtgtcagttatatggtatgatggaagaaataaagactatgcagactccgtgaagggccgattcaccatctccagagacaattccaagaacacggtgtatttggaaatgaaaggcctgagagccgaggacacggctctttattattgtgcgagagacaaaactggggattttgactcctggggccagggaaccctggtcaccgtctcctca

SEQ ID NO: 441 - protein, 117 aa, synthetic construct
QVQLVESGGGVVQPGRSLRLSCVASGFTVSSYGIHWVRQAPGKGLEWVSVIWYDGRNKDYADSVKGRFTISRDNSKNTVYLEMKGLRAEDTALYYCARDKTGDFDSWGQGTLVTVSS

SEQ ID NO: 442 - DNA, 24 bp, synthetic construct
  source 1..24: mol_type = other DNA
ggattcaccgtcagtagttatggc

SEQ ID NO: 443 - protein, 8 aa, synthetic construct
GFTVSSYG

SEQ ID NO: 444 - DNA, 24 bp, synthetic construct
  source 1..24: mol_type = other DNA
atatggtatgatggaagaaataaa

SEQ ID NO: 445 - protein, 8 aa, synthetic construct
IWYDGRNK

SEQ ID NO: 446 - DNA, 30 bp, synthetic construct
  source 1..30: mol_type = other DNA
gcgagagacaaaactggggattttgactcc

SEQ ID NO: 447 - protein, 10 aa, synthetic construct
ARDKTGDFDS

SEQ ID NO: 448 - DNA, 321 bp, synthetic construct
  source 1..321: mol_type = other DNA
gacatccagatgacccagtctccatcctccctgtctgcatctgtaggtgacagagtcaccatcacttgccgggcaagtcagaacattaacagctttttaagttggtatcagcagaaaccaggaaaagcccctaagttcctgatctatgatgcttccagtatacaaagtggggccccatcgaggttcagtggcagtggatctgggacagatttcactctcaccatcagtagtctgcaacctgaagattttgcaacttactactgtcaacagagttacagttccccgttcacttttggccaggggaccaagctggagatcaaa

SEQ ID NO: 449 - protein, 111 aa, synthetic construct
DIQMTQSPSSLSASVGDRVTITCRASQNINSFLSWYQQKPGKAPKFLIYDASSIQSGAPSRFSGSGSGTDFTLTISSLQPEDFATYYCQQSYSSPFTFGQGTKLEIKSPGK

SEQ ID NO: 450 - DNA, 18 bp, synthetic construct
  source 1..18: mol_type = other DNA
cagaacattaacagcttt

SEQ ID NO: 451 - protein, 6 aa, synthetic construct
QNINSF

SEQ ID NO: 452-453 - skipped (2 entries)

SEQ ID NO: 454 - DNA, 27 bp, synthetic construct
  source 1..27: mol_type = other DNA
caacagagttacagttccccgttcact

SEQ ID NO: 455 - protein, 9 aa, synthetic construct
QQSYSSPFT

SEQ ID NO: 456 - DNA, 375 bp, synthetic construct
  source 1..375: mol_type = other DNA
gaagtgcagctggtggagtctgggggaggcttggtacagcctggcaggtccctgagactctcctgtaaagcctctggattcgcctttgatgatttcgccatgcactgggtccggcaagctccagggaagggcctggagtgggtctcaggtattgtttggaacagtggtgacataggctatgcggactctgtgaagggccgattcaccatctccagagacaacgccaagaactccctgtatctgcaactgaatagtctgagaactgaggacacggccttgtatttctgtgtaaaagatatggttcggggacttatgggcttcaactattacggtatggacgtctggggccaagggaccacggtcaccgtctcctca

SEQ ID NO: 457 - protein, 125 aa, synthetic construct
EVQLVESGGGLVQPGRSLRLSCKASGFAFDDFAMHWVRQAPGKGLEWVSGIVWNSGDIGYADSVKGRFTISRDNAKNSLYLQLNSLRTEDTALYFCVKDMVRGLMGFNYYGMDVWGQGTTVTVSS

SEQ ID NO: 458 - DNA, 24 bp, synthetic construct
  source 1..24: mol_type = other DNA
ggattcgcctttgatgatttcgcc

SEQ ID NO: 459 - protein, 8 aa, synthetic construct
GFAFDDFA

SEQ ID NO: 460 - DNA, 24 bp, synthetic construct
  source 1..24: mol_type = other DNA
attgtttggaacagtggtgacata

SEQ ID NO: 461 - protein, 8 aa, synthetic construct
IVWNSGDI

SEQ ID NO: 462 - DNA, 54 bp, synthetic construct
  source 1..54: mol_type = other DNA
gtaaaagatatggttcggggacttatgggcttcaactattacggtatggacgtc

SEQ ID NO: 463 - protein, 18 aa, synthetic construct
VKDMVRGLMGFNYYGMDV

SEQ ID NO: 464 - DNA, 321 bp, synthetic construct
  source 1..321: mol_type = other DNA
gaaattgtgttgacgcagtctccagccaccctgtctttgtctccaggggaaagagccaccctctcctgcagggccagtcaaactattagtacttatttagtctggtaccgacagagacctggccaggctcccagtctcctcatttatgatgcatccaacagggccactgacatcccagccaggttcagtggcagtgggtctgggacagacttcactctcaccatcagcagccttgagcctgaagattttgcagtttattactgtcagcagcgtagcaactggccgatcaccttcggccaagggacacgactggagattaaa

SEQ ID NO: 465 - protein, 107 aa, synthetic construct
EIVLTQSPATLSLSPGERATLSCRASQTISTYLVWYRQRPGQAPSLLIYDASNRATDIPARFSGSGSGTDFTLTISSLEPEDFAVYYCQQRSNWPITFGQGTRLEIK

SEQ ID NO: 466 - DNA, 18 bp, synthetic construct
  source 1..18: mol_type = other DNA
caaactattagtacttat

SEQ ID NO: 467 - protein, 6 aa, synthetic construct
QTISTY

SEQ ID NO: 468-469 - skipped (2 entries)

SEQ ID NO: 470 - DNA, 27 bp, synthetic construct
  source 1..27: mol_type = other DNA
cagcagcgtagcaactggccgatcacc

SEQ ID NO: 471 - protein, 9 aa, synthetic construct
QQRSNWPIT

SEQ ID NO: 472 - DNA, 369 bp, synthetic construct
  source 1..369: mol_type = other DNA
gaagtgcagctggtggagtctgggggaggcttggtacagcctggcaggtccctgacactctcctgtgcagtctctggattcacctttgatgatcatgccatgcactgggtccggcaagctccagggaagggcctggagtgggtctcaggtattagttggaatagtgtaagtataggctatgcggactctgtgaagggccgattcacgatctccagagacaacgccaagacctccctctatctgcaaatgaacagtctgagagttgacgacacggccttatattactgtgtgcaagatttaaacgatattttgactggttatcccctctttgacttttggggccagggaaccctggtcaccgtctcctca

SEQ ID NO: 473 - protein, 123 aa, synthetic construct
EVQLVESGGGLVQPGRSLTLSCAVSGFTFDDHAMHWVRQAPGKGLEWVSGISWNSVSIGYADSVKGRFTISRDNAKTSLYLQMNSLRVDDTALYYCVQDLNDILTGYPLFDFWGQGTLVTVSS

SEQ ID NO: 474 - DNA, 24 bp, synthetic construct
  source 1..24: mol_type = other DNA
ggattcacctttgatgatcatgcc

SEQ ID NO: 475 - protein, 8 aa, synthetic construct
GFTFDDHA

SEQ ID NO: 476 - DNA, 24 bp, synthetic construct
  source 1..24: mol_type = other DNA
attagttggaatagtgtaagtata

SEQ ID NO: 477 - protein, 8 aa, synthetic construct
ISWNSVSI

SEQ ID NO: 478 - DNA, 48 bp, synthetic construct
  source 1..48: mol_type = other DNA
gtgcaagatttaaacgatattttgactggttatcccctctttgacttt

SEQ ID NO: 479 - protein, 16 aa, synthetic construct
VQDLNDILTGYPLFDF

SEQ ID NO: 480 - DNA, 324 bp, synthetic construct
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 481 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 482 - DNA, 18 bp, synthetic construct
  source 1..18: mol_type = other DNA
cagagcattagcagctat

SEQ ID NO: 483 - protein, 10 aa, synthetic construct
QSISSYSPGK

SEQ ID NO: 484-485 - skipped (2 entries)

SEQ ID NO: 486 - DNA, 30 bp, synthetic construct
  source 1..30: mol_type = other DNA
caacagagttacagtacccctccgatcacc

SEQ ID NO: 487 - protein, 10 aa, synthetic construct
QQSYSTPPIT

SEQ ID NO: 488 - DNA, 369 bp, synthetic construct
  source 1..369: mol_type = other DNA
gaagtgcagctggtggagtctgggggaggcttggtacaggctggcaggtccctaagactctcctgtgaagcctctggattcacctttgatgattatggcatgcactgggtccggcaaggtccagggaagggcctggagtgggtctcaggtattagttggaatagtggtaacatagactatgcggactctgtgaagggccgattcaccatctccagagacaacgccaagacctccctgtatctgcaaatgaacagtctgaaaactgacgacacggccttgtatttctgtgcaaaagatgctgtggggtttaactggaactactttctctttgactactggggccagggaaccctggtcaccgtctcctca

SEQ ID NO: 489 - protein, 123 aa, synthetic construct
EVQLVESGGGLVQAGRSLRLSCEASGFTFDDYGMHWVRQGPGKGLEWVSGISWNSGNIDYADSVKGRFTISRDNAKTSLYLQMNSLKTDDTALYFCAKDAVGFNWNYFLFDYWGQGTLVTVSS

SEQ ID NO: 490 - DNA, 24 bp, synthetic construct
  source 1..24: mol_type = other DNA
ggattcacctttgatgattatggc

SEQ ID NO: 491 - protein, 8 aa, synthetic construct
GFTFDDYG

SEQ ID NO: 492 - DNA, 24 bp, synthetic construct
  source 1..24: mol_type = other DNA
attagttggaatagtggtaacata

SEQ ID NO: 493 - protein, 8 aa, synthetic construct
ISWNSGNI

SEQ ID NO: 494 - DNA, 48 bp, synthetic construct
  source 1..48: mol_type = other DNA
gcaaaagatgctgtggggtttaactggaactactttctctttgactac

SEQ ID NO: 495 - protein, 16 aa, synthetic construct
AKDAVGFNWNYFLFDY

SEQ ID NO: 496 - DNA, 351 bp, synthetic construct
  source 1..351: mol_type = other DNA
caggtgcagctggtggagtctgggggaggcgtggtccagcctgggaggtccctgagactcacctgtgtagcgtctggattcaccgtcagtagttatggaatgcactgggtccgccaggccccaggcaaggggctggagtgggtggcagttatgttttatgatgaaagtaaaaaatattatgcagactccgtgaagggccgattcaccatctccagagacaattccaagaacacgctgtatctgcaaatgaacagcctgagagtcgaggacacggctgtgtattactgtgcgagagatgaacagctcgactttgaatactggggccagggaaccctggtcaccgtctcctca

SEQ ID NO: 497 - protein, 117 aa, synthetic construct
QVQLVESGGGVVQPGRSLRLTCVASGFTVSSYGMHWVRQAPGKGLEWVAVMFYDESKKYYADSVKGRFTISRDNSKNTLYLQMNSLRVEDTAVYYCARDEQLDFEYWGQGTLVTVSS

SEQ ID NO: 498 - DNA, 24 bp, synthetic construct
  source 1..24: mol_type = other DNA
ggattcaccgtcagtagttatgga

SEQ ID NO: 499 - protein, 8 aa, synthetic construct
GFTVSSYG

SEQ ID NO: 500 - DNA, 24 bp, synthetic construct
  source 1..24: mol_type = other DNA
atgttttatgatgaaagtaaaaaa

SEQ ID NO: 501 - protein, 8 aa, synthetic construct
MFYDESKK

SEQ ID NO: 502 - DNA, 30 bp, synthetic construct
  source 1..30: mol_type = other DNA
gcgagagatgaacagctcgactttgaatac

SEQ ID NO: 503 - protein, 10 aa, synthetic construct
ARDEQLDFEY

SEQ ID NO: 504 - DNA, 369 bp, synthetic construct
  source 1..369: mol_type = other DNA
gaagtgcagctggtggagtctgggggaggcttggtacagcctggcaggtccctgagactctcctgtgcagcctctggattcacctttgatgattatgccatgcactgggtccggcaagctccagggaagggcctggagtgggtctcaggtattagttggaatagtggtagcataggctatgcggactctgtgaagggccgattcaccatctccagagacaacgccaagaactccctgtatctgcaaatgaacagtctgagagctgaggacacggccttgtattactgtgcaaaagatataatggggaactgggactacttctacggtatggacgtctggggccaagggaccacggtcaccgtctcctca

SEQ ID NO: 505 - protein, 123 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDIMGNWDYFYGMDVWGQGTTVTVSS

SEQ ID NO: 506 - DNA, 24 bp, synthetic construct
  source 1..24: mol_type = other DNA
ggattcacctttgatgattatgcc

SEQ ID NO: 507 - protein, 8 aa, synthetic construct
GFTFDDYA

SEQ ID NO: 508 - DNA, 24 bp, synthetic construct
  source 1..24: mol_type = other DNA
attagttggaatagtggtagcata

SEQ ID NO: 509 - protein, 8 aa, synthetic construct
ISWNSGSI

SEQ ID NO: 510 - DNA, 48 bp, synthetic construct
  source 1..48: mol_type = other DNA
gcaaaagatataatggggaactgggactacttctacggtatggacgtc

SEQ ID NO: 511 - protein, 16 aa, synthetic construct
AKDIMGNWDYFYGMDV

SEQ ID NO: 512 - DNA, 369 bp, synthetic construct
  source 1..369: mol_type = other DNA
gaagtgcagctggtggagtctgggggaggcttggtacagcctggcaggtccctgagactctcctgtgcagcctctggattcacctttgatgataatgccatgcactgggtccggcaacctccagggaagggcctggagtgggtctcaggtattagttggaatagtggaagcataggctatgcggactctgtgaagggccgattcaccatctccagagacaacgccaagaactccctgtatctgcaaatgaacagtctgagagctgaggacacggccttgtattactgtgcaaaagatataaacgatattttgactggttatcctctttttgattactggggccagggaaccctggtcaccgtctcctca

SEQ ID NO: 513 - protein, 123 aa, synthetic construct
EVQLVESGGGLVQPGRSLRLSCAASGFTFDDNAMHWVRQPPGKGLEWVSGISWNSGSIGYADSVKGRFTISRDNAKNSLYLQMNSLRAEDTALYYCAKDINDILTGYPLFDYWGQGTLVTVSS

SEQ ID NO: 514 - DNA, 24 bp, synthetic construct
  source 1..24: mol_type = other DNA
ggattcacctttgatgataatgcc

SEQ ID NO: 515 - protein, 8 aa, synthetic construct
GFTFDDNA

SEQ ID NO: 516 - DNA, 24 bp, synthetic construct
  source 1..24: mol_type = other DNA
attagttggaatagtggaagcata

SEQ ID NO: 517 - protein, 12 aa, synthetic construct
ISWNSGSISPGK

SEQ ID NO: 518 - DNA, 48 bp, synthetic construct
  source 1..48: mol_type = other DNA
gcaaaagatataaacgatattttgactggttatcctctttttgattac

SEQ ID NO: 519 - protein, 16 aa, synthetic construct
AKDINDILTGYPLFDY

SEQ ID NO: 520 - DNA, 378 bp, synthetic construct
  source 1..378: mol_type = other DNA
gaagtgcagctggtggagtctgggggaggcgtggtacagcctggggggtccctgagactctcctgtgcagcctctacattcacctttgatgattttgccatgcactgggtccgtcaagctccagggaagggtctggagtgggtctctcttattactggggatggtggtagcacatactatgcagaccctgtgaagggccgattcaccatctccagagacaacagcaaaaactccctgtatctgcaaatgaacagtctgagaactgaggacaccgccttgtattactgtgtaaaagattggtggatagcagctcgtccggactactactactacggtatggacgtctggggccaagggaccacggtcaccgtctcctca

SEQ ID NO: 521 - protein, 126 aa, synthetic construct
EVQLVESGGGVVQPGGSLRLSCAASTFTFDDFAMHWVRQAPGKGLEWVSLITGDGGSTYYADPVKGRFTISRDNSKNSLYLQMNSLRTEDTALYYCVKDWWIAARPDYYYYGMDVWGQGTTVTVSS

SEQ ID NO: 522 - DNA, 24 bp, synthetic construct
  source 1..24: mol_type = other DNA
acattcacctttgatgattttgcc

SEQ ID NO: 523 - protein, 8 aa, synthetic construct
TFTFDDFA

SEQ ID NO: 524 - DNA, 24 bp, synthetic construct
  source 1..24: mol_type = other DNA
attactggggatggtggtagcaca

SEQ ID NO: 525 - protein, 8 aa, synthetic construct
ITGDGGST

SEQ ID NO: 526 - DNA, 57 bp, synthetic construct
  source 1..57: mol_type = other DNA
gtaaaagattggtggatagcagctcgtccggactactactactacggtatggacgtc

SEQ ID NO: 527 - protein, 19 aa, synthetic construct
VKDWWIAARPDYYYYGMDV

SEQ ID NO: 528 - DNA, 378 bp, synthetic construct
  source 1..378: mol_type = other DNA
caggtgcagctgcaggagtcgggcccaggactggtgaagccctcggagaccctgtccatcacctgcactgtctctggtggctccttcagtagtcacttctggacctggatccggcagcccccaggaaagggactggaatggattggatatctccattatagtgggggcaccagctacaacccctccctcaagagtcgagtcatcatatcagtggacacgtccaagaaccagttctccctgaaactgaactctgtgaccgctgcggacacggccgtgtattactgtgcgagagctagatcggggattacttttgggggacttatcgtccctggttcttttgatatctggggccaagggacaatggtcaccgtctcttca

SEQ ID NO: 529 - protein, 126 aa, synthetic construct
QVQLQESGPGLVKPSETLSITCTVSGGSFSSHFWTWIRQPPGKGLEWIGYLHYSGGTSYNPSLKSRVIISVDTSKNQFSLKLNSVTAADTAVYYCARARSGITFGGLIVPGSFDIWGQGTMVTVSS

SEQ ID NO: 530 - DNA, 24 bp, synthetic construct
  source 1..24: mol_type = other DNA
ggtggctccttcagtagtcacttc

SEQ ID NO: 531 - protein, 8 aa, synthetic construct
GGSFSSHF

SEQ ID NO: 532 - DNA, 21 bp, synthetic construct
  source 1..21: mol_type = other DNA
ctccattatagtgggggcacc

SEQ ID NO: 533 - protein, 7 aa, synthetic construct
LHYSGGT

SEQ ID NO: 534 - DNA, 60 bp, synthetic construct
  source 1..60: mol_type = other DNA
gcgagagctagatcggggattacttttgggggacttatcgtccctggttcttttgatatc

SEQ ID NO: 535 - protein, 20 aa, synthetic construct
ARARSGITFGGLIVPGSFDI

SEQ ID NO: 536 - DNA, 324 bp, synthetic construct
  source 1..324: mol_type = other DNA
gaaatagttttgacacagagtcccggcacactgtcactctctcccggggaaagagccaccttgtcatgtagagcaagtcagtcagtctctagctcttatctcgcctggtaccagcagaagccgggacaggcccctagactgctgatctacggggcaagttccagggccaccggaatccccgaccggttcagtggaagcggaagcggaaccgattttactttgacgatttctagactggagccagaggatttcgccgtttactattgtcaacagtacggaagcagcccgtggacgtttggccagggcacgaaggtagaaatcaag

SEQ ID NO: 537 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 538 - DNA, 21 bp, synthetic construct
  source 1..21: mol_type = other DNA
cagtcagtctctagctcttat

SEQ ID NO: 539 - protein, 7 aa, synthetic construct
QSVSSSY

SEQ ID NO: 540-541 - skipped (2 entries)

SEQ ID NO: 542 - DNA, 27 bp, synthetic construct
  source 1..27: mol_type = other DNA
caacagtacggaagcagcccgtggacg

SEQ ID NO: 543 - protein, 9 aa, synthetic construct
QQYGSSPWT

SEQ ID NO: 544 - DNA, 378 bp, synthetic construct
  source 1..378: mol_type = other DNA
caggtgcagctgcaggagtcgggcccaggactggtgaagccttcggagaccctgtccctcatttgttctgtctctggtggctccttcagtagtcacttctggagttggatccggcagcccccagggaagggactggagtggattgggtatgtcctttacagtgggggcaccaattacaacccctccctcaagagtcgagtcaccatatcagtagacacgtccaagaatcagttcttcctgaaactgagctctgtgaccgctgcggacacggccgattattactgtgcgagagctatatcggggattacgtttgggggaattatcgtccctggttcttttgatatctggggccaagggacaatggtcaccgtctcttca

SEQ ID NO: 545 - protein, 126 aa, synthetic construct
QVQLQESGPGLVKPSETLSLICSVSGGSFSSHFWSWIRQPPGKGLEWIGYVLYSGGTNYNPSLKSRVTISVDTSKNQFFLKLSSVTAADTADYYCARAISGITFGGIIVPGSFDIWGQGTMVTVSS

SEQ ID NO: 546 - DNA, 24 bp, synthetic construct
  source 1..24: mol_type = other DNA
ggtggctccttcagtagtcacttc

SEQ ID NO: 547 - protein, 8 aa, synthetic construct
GGSFSSHF

SEQ ID NO: 548 - DNA, 21 bp, synthetic construct
  source 1..21: mol_type = other DNA
gtcctttacagtgggggcacc

SEQ ID NO: 549 - protein, 7 aa, synthetic construct
VLYSGGT

SEQ ID NO: 550 - DNA, 60 bp, synthetic construct
  source 1..60: mol_type = other DNA
gcgagagctatatcggggattacgtttgggggaattatcgtccctggttcttttgatatc

SEQ ID NO: 551 - protein, 24 aa, synthetic construct
ARAISGITFGGIIVPGSFDISPGK

SEQ ID NO: 552 - DNA, 378 bp, synthetic construct
  source 1..378: mol_type = other DNA
caggtgcagctgcaggagtcgggcccaggactggtgaagccctcggagaccctgtccctcacctgcactgtctctggtggctccttcagtagtcacttctggagctggatccggcagcccccagggaagggactggagtggattggatatatcttatacactgggggcaccagcttcaacccctccctcaagagtcgagtctccatgtcagtgggcacgtccaagaaccagttctccctgaaattgagctctgtgaccgctgcggacacggccgtatattactgtgcgagagctagatcggggataacgtttacgggtattatcgtccctggctcttttgatatctggggccaagggacaatggtcaccgtctcttca

SEQ ID NO: 553 - protein, 126 aa, synthetic construct
QVQLQESGPGLVKPSETLSLTCTVSGGSFSSHFWSWIRQPPGKGLEWIGYILYTGGTSFNPSLKSRVSMSVGTSKNQFSLKLSSVTAADTAVYYCARARSGITFTGIIVPGSFDIWGQGTMVTVSS

SEQ ID NO: 554 - DNA, 24 bp, synthetic construct
  source 1..24: mol_type = other DNA
ggtggctccttcagtagtcacttc

SEQ ID NO: 555 - protein, 8 aa, synthetic construct
GGSFSSHF

SEQ ID NO: 556 - DNA, 21 bp, synthetic construct
  source 1..21: mol_type = other DNA
atcttatacactgggggcacc

SEQ ID NO: 557 - protein, 7 aa, synthetic construct
ILYTGGT

SEQ ID NO: 558 - DNA, 60 bp, synthetic construct
  source 1..60: mol_type = other DNA
gcgagagctagatcggggataacgtttacgggtattatcgtccctggctcttttgatatc

SEQ ID NO: 559 - protein, 20 aa, synthetic construct
ARARSGITFTGIIVPGSFDI

SEQ ID NO: 560 - DNA, 378 bp, synthetic construct
  source 1..378: mol_type = other DNA
caggtgcagctgcaggagtcgggcccaggactggtgaagccttcggagaccctgtccctcacttgttctgtctctggtggctccttcagtagtcacttctggagctggatccggcagcccccagggaagggactggagtggattggatatatccattacagtgggggcaccaattacaacccctccctcaagagtcgagtcaccatatcagtagacacgtccaagaaccagttctcccttaaactgacttctgtgaccgctgcggacacggccgattattactgtgcgagagctatatcggggattacgtttgggggaatgatcgtccctggttcttttgatgtctggggcgaagggacaatggtcaccgtctcttca

SEQ ID NO: 561 - protein, 126 aa, synthetic construct
QVQLQESGPGLVKPSETLSLTCSVSGGSFSSHFWSWIRQPPGKGLEWIGYIHYSGGTNYNPSLKSRVTISVDTSKNQFSLKLTSVTAADTADYYCARAISGITFGGMIVPGSFDVWGEGTMVTVSS

SEQ ID NO: 562 - DNA, 24 bp, synthetic construct
  source 1..24: mol_type = other DNA
ggtggctccttcagtagtcacttc

SEQ ID NO: 563 - protein, 8 aa, synthetic construct
GGSFSSHF

SEQ ID NO: 564 - DNA, 21 bp, synthetic construct
  source 1..21: mol_type = other DNA
atccattacagtgggggcacc

SEQ ID NO: 565 - protein, 7 aa, synthetic construct
IHYSGGT

SEQ ID NO: 566 - DNA, 60 bp, synthetic construct
  source 1..60: mol_type = other DNA
gcgagagctatatcggggattacgtttgggggaatgatcgtccctggttcttttgatgtc

SEQ ID NO: 567 - protein, 20 aa, synthetic construct
ARAISGITFGGMIVPGSFDV

SEQ ID NO: 568 - DNA, 378 bp, synthetic construct
  source 1..378: mol_type = other DNA
caggtgcagctgcaggagtcgggcccaggactggtgaagccttcggagaccctgtccctcacctgcactgtctctggtggcaccttcagtagtcacttctggagctggatccggcagcccccaggaaagggactggagtggattggatatatcttttacactgggggcaccaaccacaacccctccctcaagagtcgagtcaccatatcaatagacacgtccaagaaccagttctccctgaaactgacctctgtgaccgctgcggacacggccgtgtattactgtgcgagagctagatcggggattacgtttgggggagttatcgtccctggttcttttgatatctggggccaagggacaatggtcaccgtctcttca

SEQ ID NO: 569 - protein, 126 aa, synthetic construct
QVQLQESGPGLVKPSETLSLTCTVSGGTFSSHFWSWIRQPPGKGLEWIGYIFYTGGTNHNPSLKSRVTISIDTSKNQFSLKLTSVTAADTAVYYCARARSGITFGGVIVPGSFDIWGQGTMVTVSS

SEQ ID NO: 570 - DNA, 24 bp, synthetic construct
  source 1..24: mol_type = other DNA
ggtggcaccttcagtagtcacttc

SEQ ID NO: 571 - protein, 8 aa, synthetic construct
GGTFSSHF

SEQ ID NO: 572 - DNA, 21 bp, synthetic construct
  source 1..21: mol_type = other DNA
atcttttacactgggggcacc

SEQ ID NO: 573 - protein, 7 aa, synthetic construct
IFYTGGT

SEQ ID NO: 574 - DNA, 60 bp, synthetic construct
  source 1..60: mol_type = other DNA
gcgagagctagatcggggattacgtttgggggagttatcgtccctggttcttttgatatc

SEQ ID NO: 575 - protein, 20 aa, synthetic construct
ARARSGITFGGVIVPGSFDI

SEQ ID NO: 576 - DNA, 378 bp, synthetic construct
  source 1..378: mol_type = other DNA
caggtgcagctgcaggagtcgggcccaggactggtgaaaccttcggagaccctgtccctcacctgcactgtctctggtggctccttcagcagtcacttctggaactggatccggcagtccccagggaggggactggaatggattggatatatctattacagtgggggcaccaactataacccctccttcaagagtcgagtcaccatgtcagtagacacgtccaagaaccagttctccctgaaactgagctctgtgaccgctgcggacacggccgtgttttactgtgcgagagctagatcggggataacgtttgggggagttctcgtccctggttcttttgatatttggggccaagggacaatggtcaccgtctcttca

SEQ ID NO: 577 - protein, 126 aa, synthetic construct
QVQLQESGPGLVKPSETLSLTCTVSGGSFSSHFWNWIRQSPGRGLEWIGYIYYSGGTNYNPSFKSRVTMSVDTSKNQFSLKLSSVTAADTAVFYCARARSGITFGGVLVPGSFDIWGQGTMVTVSS

SEQ ID NO: 578 - DNA, 24 bp, synthetic construct
  source 1..24: mol_type = other DNA
ggtggctccttcagcagtcacttc

SEQ ID NO: 579 - protein, 8 aa, synthetic construct
GGSFSSHF

SEQ ID NO: 580 - DNA, 21 bp, synthetic construct
  source 1..21: mol_type = other DNA
atctattacagtgggggcacc

SEQ ID NO: 581 - protein, 7 aa, synthetic construct
IYYSGGT

SEQ ID NO: 582 - DNA, 60 bp, synthetic construct
  source 1..60: mol_type = other DNA
gcgagagctagatcggggataacgtttgggggagttctcgtccctggttcttttgatatt

SEQ ID NO: 583 - protein, 20 aa, synthetic construct
ARARSGITFGGVLVPGSFDI

SEQ ID NO: 584 - DNA, 378 bp, synthetic construct
  source 1..378: mol_type = other DNA
caggtgcagctgcaggagtcgggcccaggactggtgaagccttcggagaccctgtccctcacctgcactgtctctggtggctccttcagtagtcacttctggagctggatccggcagcccccaggaaagggactggagtggattgggtatatctattacagtgggggcacccactacaacccctccctcgagagtcgagtcaccatatcagtagacacgtccaagaaccagttctccctgaaactgaactctgtgaccgctgcggacacggccgtttattactgtgcgagagctagatcggggattacttttgggggacttatcgtccctggttcttttgatatctggggccaagggacaatggtcaccgtctcttca

SEQ ID NO: 585 - protein, 130 aa, synthetic construct
QVQLQESGPGLVKPSETLSLTCTVSGGSFSSHFWSWIRQPPGKGLEWIGYIYYSGGTHYNPSLESRVTISVDTSKNQFSLKLNSVTAADTAVYYCARARSGITFGGLIVPGSFDIWGQGTMVTVSSSPGK

SEQ ID NO: 586 - DNA, 24 bp, synthetic construct
  source 1..24: mol_type = other DNA
ggtggctccttcagtagtcacttc

SEQ ID NO: 587 - protein, 8 aa, synthetic construct
GGSFSSHF

SEQ ID NO: 588 - DNA, 21 bp, synthetic construct
  source 1..21: mol_type = other DNA
atctattacagtgggggcacc

SEQ ID NO: 589 - protein, 7 aa, synthetic construct
IYYSGGT

SEQ ID NO: 590 - DNA, 60 bp, synthetic construct; identical to SEQ ID NO: 534
  source 1..60: mol_type = other DNA

SEQ ID NO: 591 - protein, 20 aa, synthetic construct
ARARSGITFGGLIVPGSFDI

SEQ ID NO: 592 - DNA, 375 bp, synthetic construct
  source 1..375: mol_type = other DNA
cgggtgcaactggtgcagtctgggtctgaggtgaagaagcctggggcctcagtgaaggtctcctgcagggcttctggatacatcttcaccagttatgatatcaattgggtgcgacaggccactggacaagggcttgagtggatgggatggatgaaccctaataatggtaacacagcctatacacagaagttccagggcagagtcaccatgaccaggaacacctccataagcacagcctacatggagctgagcagcctgagatctgaggacacggccgtgtattactgtgcgagaaagggattactatggttcgggaagttattagggtacggtatggacgtctggggccaagggaccacggtcaccgtctcctca

SEQ ID NO: 593 - protein, 125 aa, synthetic construct
RVQLVQSGSEVKKPGASVKVSCRASGYIFTSYDINWVRQATGQGLEWMGWMNPNNGNTAYTQKFQGRVTMTRNTSISTAYMELSSLRSEDTAVYYCARKGLLWFGKLLGYGMDVWGQGTTVTVSS

SEQ ID NO: 594 - DNA, 24 bp, synthetic construct
  source 1..24: mol_type = other DNA
ggatacatcttcaccagttatgat

SEQ ID NO: 595 - protein, 8 aa, synthetic construct
GYIFTSYD

SEQ ID NO: 596 - DNA, 24 bp, synthetic construct
  source 1..24: mol_type = other DNA
atgaaccctaataatggtaacaca

SEQ ID NO: 597 - protein, 8 aa, synthetic construct
MNPNNGNT

SEQ ID NO: 598 - DNA, 54 bp, synthetic construct
  source 1..54: mol_type = other DNA
gcgagaaagggattactatggttcgggaagttattagggtacggtatggacgtc

SEQ ID NO: 599 - protein, 18 aa, synthetic construct
ARKGLLWFGKLLGYGMDV

SEQ ID NO: 600 - DNA, 324 bp, synthetic construct
  source 1..324: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccttggacgttcggccaagggaccaaggtggaaatcaaa

SEQ ID NO: 601 - protein, 108 aa, synthetic construct; identical to SEQ ID NO: 537

SEQ ID NO: 602 - DNA, 21 bp, synthetic construct
  source 1..21: mol_type = other DNA
cagagtgttagcagcagctac

SEQ ID NO: 603 - protein, 7 aa, synthetic construct
QSVSSSY

SEQ ID NO: 604-605 - skipped (2 entries)

SEQ ID NO: 606 - DNA, 27 bp, synthetic construct
  source 1..27: mol_type = other DNA
cagcagtatggtagctcaccttggacg

SEQ ID NO: 607 - protein, 9 aa, synthetic construct
QQYGSSPWT

SEQ ID NO: 608 - protein, 120 aa, synthetic construct; identical to SEQ ID NO: 360

SEQ ID NO: 609 - protein, 8 aa, synthetic construct
GFTFSAYA

SEQ ID NO: 610 - protein, 8 aa, synthetic construct
ISGSGGSA

SEQ ID NO: 611 - protein, 13 aa, synthetic construct
AKDGAWKMSGLDV

SEQ ID NO: 612 - protein, 107 aa, synthetic construct; identical to SEQ ID NO: 368

SEQ ID NO: 613 - protein, 6 aa, synthetic construct
QDISDY

SEQ ID NO: 614 - skipped

SEQ ID NO: 615 - protein, 9 aa, synthetic construct
QKYDSAPLT

SEQ ID NO: 616 - protein, 109 aa, Homo sapiens; identical to SEQ ID NO: 340

SEQ ID NO: 617 - protein, 426 aa, Homo sapiens
MPLLWLRGFLLASCWIIVRSSPTPGSEGHSAAPDCPSCALAALPKDVPNSQPEMVEAVKKHILNMLHLKKRPDVTQPVPKAALLNAIRKLHVGKVGENGYVEIEDDIGRRAEMNELMEQTSEIITFAESGTARKTLHFEISKEGSDLSVVERAEVWLFLKVPKANRTRTKVTIRLFQQQKHPQGSLDTGEEAEEVGLKGERSELLLSEKVVDARKSTWHVFPVSSSIQRLLDQGKSSLDVRIACEQCQESGASLVLLGKKKKKEEEGEGKKKGGGEGGAGADEEKEQSHRPFLMLQARQSEDHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 618 - protein, 340 aa, synthetic construct; identical to SEQ ID NO: 391

SEQ ID NO: 619 - protein, 411 aa, Homo sapiens
MDGLPGRALGAACLLLLAAGWLGPEAWGSPTPPPTPAAQPPPPPPGSPGGSQDTCTSCGGFRRPEELGRVDGDFLEAVKRHILSRLQMRGRPNITHAVPKAAMVTALRKLHAGKVREDGRVEIPHLDGHASPGADGQERVSEIISFAETDGLASSRVRLYFFISNEGNQNLFVVQASLWLYLKLLPYVLEKGSRRKVRVKVYFQEQGHGDRWNMVEKRVDLKRSGWHTFPLTEAIQALFERGERRLNLDVQCDSCQELAVVPVFVDPGEESHRPFVVVQARLGDSRHRIRKRGLECDGRTNLCCRQQFFIDFRLIGWNDWIIAPTSYYGNYCEGSCPAYLAGVPGSASSFHTAVVNQYRMRGLNPGTVNSCCIPTKLSTMSMLYFDDEYNIVKRDVPNMIVEECGCASPGK

SEQ ID NO: 620 - protein, 122 aa, Homo sapiens
EVQLLESGGGLVQPGGSLRLSCAASGFTFSSFAMSWVRQAPGKGLEWVSTISGSGGYTFYADSVKGRFTISRDNSKNTLYLQMNSLRAEDTAVYYCAKDGRYNWNYGAFDIWGQGTMVTVSS

SEQ ID NO: 621 - protein, 107 aa, Homo sapiens
EIVMTQSPATLSVSPGERATLSCRASQSVSSNLAWYQQKPGQAPRLLIYGASTRATGIPARFSGSGSGTEFTLTISSLQSEDFAVYYCQQYNNWPLTFGGGTKVEIK

SEQ ID NO: 622 - protein, 115 aa, synthetic construct
  source 1..115: note = Synthetic 12A5-5HC polypeptide
  VARIANT 50: note = any naturally occurring amino acid
EVQLVESGGGLVQPGGSLRLSCAASGFTFSRYWMNWVRQAPGKGLEWVAXIRLKSDNYATHYAESVKGRFTISRDNAKNSLYLQMNSLRAEDTAVYYCTEGLDYWGQGTTVTVSS

SEQ ID NO: 623 - protein, 107 aa, synthetic construct
  source 1..107: note = Synthetic 12A5-5LC polypeptide
DIQMTQSPSSLSASVGDRVTITCKASQDINKYVAWYQQKPGKAPKLLIYYTSWLQPGVPSRFSGSGSGTDFTFTISSLQPEDIATYYCLQYDNLLYTFGQGTKLEIK

SEQ ID NO: 624 - protein, 124 aa, synthetic construct
  source 1..124: note = Antibody Fragment
QVQLVQSGAEVKKPGASVKVSCKASGYSFTGYFMHWVRQAPGQGLEWIGNIYPYNSVSNYNQRFKARATLTVDKSTSTAYMELRSLRSDDTAVYYCARRYYYGTGPADWYYDVWGQGTLVTVSS

SEQ ID NO: 625 - protein, 107 aa, synthetic construct
  source 1..107: note = Antibody Fragment
DIQMTQSPSSLSASVGDRVTITCKASQDINSYLSWFQQKPGKAPKTLIYRANRLVDGVPSKFSGSGSGTDYTLTISSLQPEDFATYYCLQSDEFPLTFGQGTKLEIK

SEQ ID NO: 626 - protein, 117 aa, Homo sapiens
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDENWGFDPWGQGTLVTVSS

SEQ ID NO: 627 - protein, 105 aa, Homo sapiens
SYELTQPPSVSVSPGQTATITCSGHALGDKFVSWYQQGSGQSPVLVIYDDTQRPSGIPGRFSGSNSGNTATLTISGTQAMDEADYFCQAWDSSFVFGGGTKVTVL

SEQ ID NO: 628 - protein, 120 aa, Homo sapiens
QVQLVQSGAEVKKPGASVKVSCKASGYTFTSYWMQWVRQAPGQGLEWMGAIYPGDGDTRYTQKFKGRVTMTRDTSTSTVYMELSSLRSEDTAVYYCARMGGYDRYYFDYWGQGTLVTVSS

SEQ ID NO: 629 - protein, 113 aa, Homo sapiens
DIVMTQSPDSLAVSLGERATINCKSSQSLLNSANQKNYLAWYQQKPGQPPKLLIYFASTRESGVPDRFSGSGSGTDFTLTISSLQAEDVAVYYCQQHYNTPLTFGGGTKVEIK

SEQ ID NO: 630 - protein, 113 aa, Homo sapiens
  VARIANT 1: note = unknown amino acid
  VARIANT 113: note = unknown amino acid
XVKLQQSGAELVKPGASVKLSCKASGYTFTSFYMYWVKQRPGQGLEWIGEINPSNGDTNFIESFKSKATLTVDKSSSTAYMQLSSLTSEDSAVYYCTVRFAYWGQGTTVTVSX

SEQ ID NO: 631 - protein, 112 aa, synthetic construct
EVQLLESGGGLVQPGGSLRLSCAASGFTFSSYAMSWVRQAPGKGLEWVSTISSGGSYTSYPDSVKGRFTISRDNSKNTLYLQMNSLRAEDTAVYYCAKQDYAMNYWGQGTMV

SEQ ID NO: 632 - protein, 107 aa, synthetic construct
DIQMTQSPSSLSASVGDRVTITCKASQDVSTAVAWYQQKPGKAPKLLIYSASYRYTGVPSRFSGSGSGTDFTLTISSLQPEDFATYYCQQHYSTPWTFGQGTKVEIK

SEQ ID NO: 633 - protein, 116 aa, synthetic construct
  source 1..116: note = Mouse CDRs and human VH & J segment
EVQLLESGGGLVQPGGSLRLSCAASGFTFSSYAMSWVRQAPGKGLEWVSTISSGGSYTSYPDSVKGRFTISRDNSKNTLYLQMNSLRAEDTAVYYCAKQDYAMNYWGQGTLVTVSS

SEQ ID NO: 634 - protein, 107 aa, synthetic construct
  source 1..107: note = Mouse CDRs and human VH & J segment
DIQMTQSPSSLSASVGDRVTITCKASQDVSTAVAWYQQKPGKAPKLLIYSASYRYTGVPSRFSGSGSGTDFTLTISSLQPEDFATYYCQQHYSTPWTFGGGTKVEIK

SEQ ID NO: 635 - protein, 124 aa, synthetic construct
QLQLQESGPGLVKPSETLSLTCTVSGFSLRKVGSSVSWIRQPPGKGLEWIGHIYWDDDKRLNPSLRNRVTISVDTSKNQFSLKLSSVTAADTAVYYCARRAITTVIGGGTFDLWGQGTLVTVSS

SEQ ID NO: 636 - protein, 106 aa, synthetic construct
DIQMTQSPSSLSASVGDRVTITCSASSSISYMHWYQQKPGKAPKLLIYDTSKLARGVPSRFSGSGSGTDFTLTISSLQPEDFATYYCQQWYLNPLTFGGGTKVEIK

SEQ ID NO: 637 - protein, 124 aa, synthetic construct
QVTLRESGPALVKPTQTLTLTCTFSGFSLRKVGRSVSWIRQPPGKALEWLAHIYWDDDKRYNPSLRNRLTISKDTSKNQVVLTMTNMDPVDTATYYCARRAITTVIGGGTFDYWGQGTTVTVSS

SEQ ID NO: 638 - protein, 106 aa, synthetic construct
DIQMTQSPSSLSASVGDRVTITCSASSSISYMHWYQQKPGKAPKLLIYDTSKLARGVPSRFSGSGSGTDFTLTISSLQPEDFATYYCQQWYRNPLTFGGGTKVEIK

SEQ ID NO: 639 - protein, 113 aa, synthetic construct
EVQLVESGGGLVQPGGSLRLSCAASGLTFSRYPMSWVRQAPGKGLVWVSAITSSGGSTYYSDTVKGRFTISRDNAKNTLYLQMNSLRAEDTAVYYCARLPDYWGQGTLVTVSS

SEQ ID NO: 640 - protein, 112 aa, synthetic construct
EIVLTQSPGTLSLSPGERATLSCRASSSVSSSYLHWYQQKPGQAPRLLIYSTSNLVAGIPDRFSGSGSGTDFTLTISRLEPEDFAVYYCQHHSGYHFTFGGGTKVEIKSPGK

SEQ ID NO: 641 - protein, 439 aa, synthetic construct
  source 1..439: note = Full length landogrozumab heavy chain
EVQLVESGGGLVQPGGSLRLSCAASGLTFSRYPMSWVRQAPGKGLVWVSAITSSGGSTYYSDTVKGRFTISRDNAKNTLYLQMNSLRAEDTAVYYCARLPDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 642 - protein, 215 aa, synthetic construct
  source 1..215: note = Full length landogrozumab light chain
EIVLTQSPGTLSLSPGERATLSCRASSSVSSSYLHWYQQKPGQAPRLLIYSTSNLVAGIPDRFSGSGSGTDFTLTISRLEPEDFAVYYCQHHSGYHFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 643 - protein, 7 aa, synthetic construct
  source 1..7: note = HCVR1
GLTFSRY

SEQ ID NO: 644 - protein, 6 aa, synthetic construct
  source 1..6: note = HCVR2
TSSGGS

SEQ ID NO: 645 - protein, 4 aa, synthetic construct
  source 1..4: note = HCVR3
LPDY

SEQ ID NO: 646 - protein, 9 aa, synthetic construct
  source 1..9: note = LCVR1
SSVSSSYLH

SEQ ID NO: 647 - protein, 7 aa, synthetic construct
  source 1..7: note = LCVR2
STSNLVA

SEQ ID NO: 648 - protein, 9 aa, synthetic construct
  source 1..9: note = LCVR3
QHHSGYHFT